SEQUENCE LISTING (2 sequences)

SEQ ID NO: 1 - DNA, 16569 bp, Artificial Sequence
  SEQ ID NO1 is a composite sequence of the cambridge sequence and human mitochondrial DNA sequence alleles.
gatcacaggtctatcaccctattaaccactcacgggagctctccatgcatttggtattttcgtytggggggyrtgcacgcgatagcatygcgrgmcgctggagccggagcaccytatgtcgcagtatctgtctttgattcctrccycatyyyrttatttatcgcacctacrttcaatatyayrgdmgavcatayhtayyraagygtrytraytartyaatgcttrtrrgacataryaataacaattraaygyctgcacagccrctttccacacagacatcataacaaaaartttycrccaaaccccccctcccccrvttytggcyacagcacttaaacayatctctgccaaaccccraaaacaaagaaccctracaccagcctaaccagatttcaaattktatctttwggcggtatgyacttttaacagtcaccccccaactaacacattattttyccctcycaytyccayactactaaycycatcaayacarcccccrcccatcctrcccagcacacacacaccgctgctaaccccataccccgaaccaaccaaaccccaaagacaccccccacagtttatgtagcttaccycctyaaagcaatacactgaaaatgtttagacgggctcacatcaccccataaacaaataggtttggtcctrgcctttctattagcycytagtaagattacacatgcaagcatccccrytccagtgagtycaccctctaaatcaccacgatcaaaaggracaagcatcaagcacgcarcaatgcagctcaaaacgcttagcctagccacacccccacgggaaacagcagtgatwarcctttagcaataaacgaaagttyaactaagctatactaaccccagggttggtcaatttcgtgccagccaccgcggtcacacgattaacccaagycaatagaarccggcgtaaagagtgttttagatcacccccbccccaataaagctaaaactcacctgagttgtaaaaaactccagttgacacaaaataractacgaaagtggctttaacatrtctgaayacacaatagctaagacccaaactgggattagataccccactatgcttagccctaaacctcaacagttaaaycaacaaaactgctcgccagaacactacgagccacagcttaaaactcaaaggacctggcggtgcttcatayccctctagaggagcctgttctgtaatcgataaaccccgatcaacctcaccaccycttgctcagcctatataccgccatcttcagcaaaccctgatgaaggytacaaagtaagcgcaagtacccacgtaaagacgttaggtcaaggtgtagcccatgaggtggcaagaaatgggctacattttctaccccagamaactacgatagcccttatgaaacytaagggtcraaggyggatttagcagtaaactragartagagtgcttagttgaacagggccctgaagcgcgtacacaccgcccgtcaccctcctcaartatacttcaaaggacatttaactaaaacccctacgcatttatatagaggagacaagtcgtaacatggtaagtgtactggaaagtgcacttggacraaccagagtgtagcttaacacaaagcacccaacttacacttaggagatttcaacttaacttgaccgctctgagctaaacctagccccaaacccactccaccytaytaycaracaacyttarccaaaccatttacccarayaaagtataggcgatagaaattgaaacctggcgcaatagatayagtaccgcaagggaaagatgaaaaattatarccaagcataatatagcaaggactaacccctataccttctgcataatgaattaactagaaataactttgcaaggagarccaaagctaagacccccgaaaccagacgagctacctaaraacagctaaaagagcacacccgtctatgtagcaaaatagtgggaagatttataggtagaggcgacaaacctaycgagcctggtgatagctggttgtccaagatagaatcttagttcaactttaaatttgcccrcagaaccctctaaatccccttgtaaatttaaytgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatatvactgaactcctcacaccmaattggaccaatctatcaccctatagaagaactaatgttagtatragtaacatgaaaacattctcctcygcataagcctgcgtcagatyaaaacrctgaactgacaattaacagccyaatatctacaatcaaccaacaagtcattattacccycactgtcaacccaacacaggcatgctcataaggaaaggttaaaaaaagtaaaaggaactcggcaaaycttaccccgcctgtttaccaaaaacatcacctctagcatcaccagtattagaggcaccgcctgcccagtgacacatgtttaacggccgcggtaccctraccgtgcaaaggtagcataatcacttgttccttaaatagggacctgtatgaatggctycacgagggttyagctgtctcttacttttaaccagtgaaattgacctgcccgtgaagaggcgggcatracacagcaagacgagaagaccctatggagctttaatttattaatgcaarcartacctaacaracccacaggtcctaaactacyaarcctgcattaaaaatttcggttggggcgacctcggagcagaaycmaacctccgagcagtacatgcyaagacytcaccagtcaaagcgaactacyatactcaattgatccaataacttgaccaacggaacaagttaccctagggataacagcgcaatcctattctagagtccatatcaacaatagggtttacgacctcgatgttggatcaggacatcccratggtgcagccgctattaaaggttcgtttgttcaacgattaaagtcctacgtgatctgagttcagaccggagyaatccaggtcggtttctatctancttcaaattcctccctgtacgaaaggacaagagaaataaggcctacttcacaaagcgccttcccccgtaaatgatatcatctcaacttagyatwayaycyacacccacccaagarcagggtttgttaagatggcagagcccggtaatcgcataaaacttaaaactttacagtcagaggttcaaytcctcttcttaacaacayacccatgrccaacctcctactcctcattgtacccattctaatcgcaatggcattcctaatgctyaccgaacgaaaaattctaggcyatatacaactacgcaaaggccccaacgttgtaggcccctacggrctactacaaccyttcgctgacgccataaaactcttcaccaargagcccctaaaacccgccacatctrccatcacyctvtacatcaccgccccgaccttrgctctcaccatygchcttctactatgarcccccctccccatacccaaccccctggtyaacctcaacctaggcctcctatttattctagccacctctagcctagccgyttactcaatcctctgatcaggrtgagcatcaaactcaaactacgccctratcggygcactgcgagcagtagcccaracaatctcatatgaagtcaccctagccatcattctrctatcaacattactaataagtggctcctttaacctctccncccttatcacarcacaagarcacctctgattactcctrccatcatgrcccytggccataatatgatttayctccacactagcagagaccaaccgaacccccttcgaccttgccgaaggggartcmgaactrgtctcaggcttcaacatcgaatacgccgcaggccccttcgccytattcttcatrgccgaatacacaaacattattataataaacaccctcaccactayaatcttcctaggaayaacrtatracgcactctcccctgaactctacacaacatattttgtyaccaagaccctacttctracctccctgttcytatgrrttcgaacagcatacccccgattccgctacgaccarctcatacacctcctatgaaaaaacttcctaccactcaccctagcrttacttatatgayatgtytccrtacccaytacaatctccagcatyccccctcaaacctaagaaatatgtctgataaaagagttactttgatagagtaaataataggagyttaaacccccttatttctaggacyatgagaatcgaacccatccctgagaatccaaaaytctccgtgccacctatcrcaccccatcctaaagtaaggtcagctaaataagctatcgggcccataccccgaaaatgttggttawacccttcccgtactaattaatcccctggcccaacccrtcatctactctaccrtytttrcaggcacactcatcachgcgctaagctcrcactgattttttacctgagtaggcctagaaataaacatrctagcytttattccarttctaaccaaaaaaataaaccctcgttccacagaagctgccatcaagtayttcctcacrcaagcaaccgcatccataatccttctaatagcyatcctcytcaacaatatactctccggrcaatgaaccataaccaatactaccaaycaatactcatcattaataatcatartrgctatagcaataaaactaggaatagccccctttcacttctgagtcccagargttrcccaaggcrcccctctracatccggcctgcttcttctcacatgacaaaaactagccccyatctcaatcatataccaaatctcyccctcactaracgtaagccttctcctcactctctcaatcttatccatcatagyaggcagttgaggtggaytaaaccaaacccagctrcgcaaaatcytagcatactcctcaattacccayataggatgrataatakcarttctaccgtacaacccyaacataaccattcttaatttaactatttatatyatcctaactacyaccgcattcctactactcaacttaaactccagcaccacraccctrctactatctcgcacctgaaacaagmtaacatgactaacacccttaattccatccaccctcctctccctaggaggcctrcccccrctaaccggctttttgccyaaatggrycattatcgaagaattcacaaaraacaatagcctcatyatccccaccatcatagccaccatcaccctmmttaacctctacttctacctacgcctaatctactccacctcaatcacactactccccatrtcyaacaacgtaaaaataaaatgacartttgaacayacaaaacccaccccaytcctccccacactcatcrccctyaccacrctactcctacctatctccccyttyatactaataatcttatagaaatttaggttaaatacagaccaagagccttcaaagccctcagtaagttgcaatacttaatttctgyracagctaaggactgcaaaaycycaytctgcatcaactgaacgcaaatcagcyactttaattaagctaagccctyactagaccaatgggacttaaacccacaaacacttagttaacagctaagcaccctartcaactggcttcaatctacttctcccgccgccgggaaaaaaggcgggagaagccccggcagrtttgaagctgcttcttcgaatttgcaattcaatatgaraaycacctcrgagcyggtaaaaagaggcctarcccctgtctttagatttacagtccaatgcttcactcagccattttacctcacccccactgatgttcgccgaccgttgactattctctacaaaccacaaagacattggracactatacctattattcggcgcatgagctggrgtyctaggcacagctctaagcctccttattcgagccgagctrggycagccaggcaaccttytaggtaacgaccacatctacaacgtyatcgtyacagcccatgcatttgtaataatcttyttcatagtaatacccatcataatcggaggctttggcaactgactartycccctaataatyggygcccccgatatggcrttyccccgcataaacaacataagcttctgactcttaccyccctcyctcctactcctgctcgcatctgctayagtrgaggccggagcaggaacaggttgaacagtctaccctcccttagcagggaactactcccaccctggarcctccgtagacctaaccatcttctccttacacctagcaggtrtctcytctatcttaggggccatcaayttcatcacaacaattatcaatataaaaccccctgccataacccaataccaaacgcccctcttcgtctgatccgtcctaatyacagcagtcctacttctmctatctctcccagtcctagctgctggcatcacyatactactaacagaccgcaacctyaacaccaccttcttcgaccccgccggaggaggagacccyattctataccaacacctatyctgatttttcggtcaccctgaagtttatattcttatcctaccaggcttcggaataatctcccatattgtaacytactactccggaaaaaaagaaccatttggatayataggyatggtctgagctatratatcaattggcttcctrgggtttatcgtgtgagcrcaccayatatttacagtaggaatagacgtagacacacgagcatayttcacctccgcyaccataatcatcgctatccccaccggcgtcaaagtatttagctgactmgccacactccacggaagcaatatgaaatgatctgctgcagtgctctgagccctaggattcatytttcttttcaccgtaggtggcctractggcattgtattagcaaactcatcrctagacatcgtactacacgacacgtactacgttgtagcycacttccactatgtcctatcaatrggrgcwgtatttgccatcataggrggcttcattcactgatttcccctattctcaggctacaccctagaccaaacctacgccaaaatccatttcrctatcatrttcatcggcgtaaatctaacyttcttcccacaacactttctmggcctrtccggaatgccccgacgttactcrgactaccccgatgcatacaccacatgaaayrtcctatcatctgtrggytcattcatttctctaacagcagtaatattaataattttcatgatytgagaagccttcgcttcraagcgaaaartcctaatagtagaagaaccctccataaacctggagtgactayatggatgccccccrccctaccacacattcgaagarcccgtatacataaaatctaracaaaaaaggaaggaatcgaaccccccaaagytggtttcaagccaaccycatggcctccatgactttttcaaaaagrtattagaaaaaccatttcataactttgtcaaagttaaatyataggctaartcctatatatcttaatggcacatgcagcrcaagtaggtctacaagacgctacwtcccctatcatagaagagctyatyacctttcatgaycacrccctcatartyattttccttatctgcttyytartcctgtatgcccttttcctaacactcacaacaaaactaactaatacyaacatctcagacgctcaggaratrgaraccgtctgaactatcctgcccgccatcatcctagtcctcatcgccctcccatccctacgcatcctttacataacagacgaggtcaaygayccytcycttaccatcaaatcaattggccaccaatggtactgaacctacgagtacaccgactacggcggactratcttcaactcctayatacttcccccattattcctagaaccaggcgacctgcgactccttgacgtygacaatcgagtagtrctcccrattgaarcccccattcgtataataattacatcacaagacgtcttgcactcatgagctgtycccacaytaggcttaaaaacagatgcaattccmggacgtctaaaccaaaccactttcaccgytacacgrccrggrgtatactacggtcaatgctctgaaatctgyggagcaaaccacagyttcatrcccatcgtcctagaattaattcccctaaaaatctttgaaatrggrcccgtattyaccctatarcaccccctctacccccyctagarcccacygtaaagctaacttagcattaaccttttaagttaaagattaagagarccaacacctctttacagtgaaatgccccaactaaatactaccrtrtgrcccaccataatyacccccataytccttacactattyctcatcacccaactaaaaayattaaacacaarctaccacytacyyccctcaccaaarcccataaaaataaaaaattataacaaaccctgagaaccaaaatgaacgaaaatctgttcrcttcattyattgcccccrcartcctaggcctrcccgccrcagtactgatcattctatttccccctctattgayccccacctccaaatatctcatcaacaaccgactaatyaccacccaacaatgactaatcaaactaacctcaaaacaaatratarcyayacayaacactaaaggrcgaacctgatcycttatactagtatccttaatcatttttattrccacaactaacctcctmggrctcctrccyyactcatttacrccaaccacccaactatctataaacctagccrtrgccatccccttatgagcrggcrcagtgattataggcytycgctctaagattaaaaatgccctagcccacttcytrccacaaggcacaccyacaccccttatcccyatactagttattatcgaarccatcagcctactcattcaaccaatagccctrgccgtacgcctaaccgctaacattactgcaggccacctactcatgcayctaattggaarcrccaccctagcaatatcraccaytaaccttccctcyacmcttatcatcytcacaattctrattctrctractatcctagaartcgctgtcgccttartccargcctacgttttcacactyctagtaagcctctacctgcacgacaacacataatgacccaccaatcrcatgcctatcatatartaaarcccagyccatgacccctaacrggggccctytcagccctcctaatgacctccggyctagccatgtgattycacttccactccayaacgctcctyatactaggcctrctaaccaryacactaaccatataccaatgrtggcgcgatgtaacacgagaaagcmcataccaaggccaccacacaccacctgtccaaaaaggccttcgataygggatartcctatttattacctcagaartttttttcttcgcaggatttttctgagccttytaccactccagcctagcccctaccccycaaytaggrggrcactgrccccsaacaggcatcaccccrctaaatcccctagaartcccactyctaaacacatccgtattactcgcatcaggagtrtcaatcacctgagcycaccatagtctaatagaaarcaaccgaaaccaaayaattcaagcactgctyattacaattttactgggtctctattttaccctcctacaagcctcagagtacttcgartctcccttcaccatttccgacggcatctacggctcaacattttttgtagccacaggcttccayggamtwcacgtcattattggctcaactttcctcactatctgcttcatccgccaactaatatttcactttacatccaaacatcactttggcttygaagccgccgcctgatactgrcattttgtagatgtggtytgactayttctgtatrtctccatctaytgatgagggtcttactcttttagtataaatagtaccgttaacttccaattaactagytttgacaacattcaaaaaagagtaataaacttcgccttaattttaataatcvacaccctcctagccttactactaataatyatyacattttgactaccacaactcaayggctacatrsaaaaatccaccccttacgartgcggcttcsaccctatatcccccrcccgcgtccctttctccataaaattcttcttagtagctatyaccttcttattattygayctagaaattgccctccttttacccctaccatgagccctacaaacaactaacctrccrctaatagytatrtcatccctcttattaatcatcatcctagccctragtctggcctaygagtgactacaaaaaggattagactgarcygaattggtayataktttaaacaaaacraatgatttcgactcattaaattatgataatcatatytaccaaatgcccctcatttacataaatattatactrgcattyaccatctcacttctaggaatactagtatatcgctcacacctcatrtcctccctactatgcctagaaggaataatactatcrctrttcattatagctactctcayaaccctcaacacccactccctcttagcyaayattgtrcctattgccataytagtyttygccgcctgcgaagcagcggtrggcctagccctactagtctcaatctccaacacatatggcctagactaygtacataacctaaacctactccaatgctaaaactaatcgtcccaacaattataytrytaccactgacrtgactytccaaaaarcacataatytgaatcaacacaaccacccacagcctaattattagcatcatcccyctrctattttttaaccaaatyaacaacaacctatttagctgytcccyaaccttttcctccgacccyctaacaacccccctcctaatactaacyacctgactcctacccctsacaatcatggcaagccarcgccacttatccarygaaccrctatcacgaaaaaaactctacctctctatactaatctccctacaaatctccttarttataacattcacrgccacagaactaatcatattttatatcttcttcgaaaccacacttatccccaccytgrctatcatcacccgatgrggcarccarycagaacgcctgaacgcaggcacatacttcctattctayaccctagtaggctcccttcccctactcatcgcactratttayactcacaacaccctaggctcactaaacattctactactyacyctcactgcccaagaactatcaaactcctgagcyaacaacttaatatgactagcttacacaatrgcytttatagtaaaratacctctttacggactccacttatgactccctaaagcccatgtcgaagcccccatcgctgggtcaatagtacttgccgcagtactcttraaactaggyggctatggtataatacgcctcacactcattctcaaccccctgacaaaacacatagcctaycccttccttgtactatccctatgaggcataattataacaagctccatctgcctrcgacaaacagacctaaaatcrctcattgcatactcttcaatcagccacatrgccctcgtagtrrcagccattctcatccaaacyccctgaagcttcaccggcgcagtcatyctcataatcgcccacggrctyacatcctcattactattctgcctagcaaactcaaactacgaacgyactcacagtcgcatcataatcctctctcaaggacttcaaactctrctcccactaatagctttttgatgacttctagcaagcctcgcyaacctcgccttaccccccactattaacctrctrggagarctctcygtgctagtarccacrttctcctgatcaaatatcactctcctactyacrggactcaacatrctartcacarccctatactccctctacatatttaccacaacacaatgrggctcactcacccaccacattaacaacataaaaccctcattyacacgagaaaacaccctcatrttcatacacctakcccccattctcctcctatccctcaaccccgacatcatyaccgggttttccycttgtaaatatagtttaaycaaaacatcagattgtgartcygacaacagaggcttacgaccccttatttaccgagaaagctcacaagaactgctaactcrtrccyccatgtctracaacatggctttctcaacttttaaaggataacagctatccattggtcttaggccccaaraattttggtgcaactccaaataaaagtaataaccatgyacactactatarccrccctaaccctrrcttccctaattccccccatccttrccaccctcrttaacccyaacaaaaaaaactcatacccccattatgtaaaatccattgtcgcatccacctttattatcagyctcttccccacaacaatattcatrtgcctrgaccaagaagtyattatctcraactgacactgrgccacaacccaaacaacccagctctccctaagcttcaaactagactacttctccataatattcatccctgtrgcattgttcgttacatgrtcyaycatagaattctcactgtgatatataaactcagayccraacattaatcagttcttcaartatctactcatyttcctaattaccatrctaatcttagttaccgcyaacaacctattccaactgttcatcggctgrgarggcgtaggaattatatccttcttgctcatcagttgatgrtacgcccgagcrgatgccaacacagcagccattcaagcartcctatacaaccgtatcggcgatatcggyttyatcctcgccttagcatgatttatcctacactccaactcatgagacccwcaacaaatarcccttctraacgctaatccaagcctcmccccrctactaggcctcctcctagcagcagcrggcaaatcagcccaattaggyctccacccctgactcccctcagccatagaaggccccacyccagtctcrgccctactccactcaagcactatagttgtagcmggrrtcttcttactcatccgcttccaccccctarcagaaaayarcccrctaatccaaactctaacactatgcttaggcgctatcaccactctrttygcagcagtctgcgcycttacacaraatgacatcaaaaaaatcgtagccttctccacttcaagtcarctaggactcatartagtyacaatcggcatcaaccaaccacacctagcattcctgcacatctgtacccacgccttcttcaaagccatactatttatgtgctccggrtccatcatccacaaccttaacaatgaacaagatattcgaaaaataggaggactactcaaaaccatacctctsacttcaacctccctcaccattggcagcctagcattarcaggaatrcctttcctyacaggbttctaytccaargaccacatcatcgaaaccgcaaacatatcatacacaaacgcctgagccctrtctattactctcatcgctacctccctracargcgcctayagcactcgrataatycttctcaccctaacaggtcaaccycgcttcccyrcccttactracattaacgaaaataaccccaccctactaaaccccattaaacgcctgrcagccggaagcctrttcgcaggattyctcattactaacaacatttcccccrcatcccccttccaaacaacartccccctcyacctaaaactcacrgccctcgcygtcacyytcctaggrcttctaacagccctagacctcaactacctaaccaacaaacttaaaataaaatmcccacyatgcacattttatttctccaacatactmggattctacyctwscatcacacaccgcacaatcccctatctagsccttctyrcgagccaaaacctrcccctactcctcctagaccwaacctgactagaaaarctaytrccyaaaacaatytcacagcaccaaatctccacctccrtcatcacctcdacccaaaaaggcataatyaaactytayttcctctctttcttcttcccrctcatcctarccctactcctaatcacatarcctrttcccccgagcaatytcaattacaayatayacaccaacaaacaatgtycarccagtracyacyactaaycaacgcccatartcatacaaagcccccgcaccaataggatcctcccgaatsaaccctgacccytctccttcataaattattcagctyccyacactayyaaagtttaccacaaccaccaccccatcatactctttcacccacagcaccaayccyacctccatcsctaaccccactaaaacactcaccaagacctcaacccctgacccccatgcctcaggatactcctcaatagcyatcrctgtagtatayccaaagacaaccaycatyccccctaaataaaytaaaaaaactattaaacccatataacctcccccaaaattcagaataataacacacccraccacrccrcwaacaatcartactaarcccccataaataggagarggcttagaagaaaaccccacaaaccccattactaaacccacactcaacagaaacaaagcatayatcattattctcgcacggactacarccacgaccaatgatatgaaaaaccatcgttgtatttcaactacaagaacaccaatgaccccaatacgcaaaaytarccccctaataaaaytaattaaccrctcaytcatcgacctcccyaccccatccaacatctccgcatgrtgaaacttcggctcactccttggcryctgcctgatcctccaaatcaccacaggactattcctagccatrcactaytcaccagacgcctcaaccgccttttcatcaatcgcccacatcactcgagacgtaaattayggstgaaycatccgctaccttcacgccaatggcgcctcaatattytttatctgcctcttcctrcacatcggrcgaggcctatattacggatcatttctctactcagaaacctgaaacatcggcattatcctcctgcttrcarcyatagcaacagccttcataggytatgtcctcccgtgaggccaaatatcattctgaggrgccacagtaattacaaacttactatccgccaycccatacattggrmcagacctagtycaatgaatstgaggrggctactcagtaracartcccaccctcacacgattctttacctttcacttcatcttrcccttcattattgcarycctarcarcactccacctcctattcttrcacgaaacgggrtcaaacaaccccctaggaatcacctcccattccgataaaatcaccttccacccttactacacaatcaaagacrccctcggcttrcttctcttcmttctctccttaatracaytaacactattctcaccwgacctcctargcgacccagacaattayacccyagccaaccccttaaayacccctccccacatcaagcccgaatgatatttcctattcgcctacacaattctccgatccgtccctaacaarctaggaggcgtccttgccytaytactatccatcctcatyctagcaataatcccyaycctccayatatccaaacaacaaagcataatatttcgcccactaagccaatcactttattgrctcctarccgcagacctcctcrttctaacctgaatcggaggrcaaccagtaagctacccytttaccatyattggacaartarcatccrtactatacttcrcaacaatcytaatcctaataccaaytatctccctaattgaaaacaaaatactcaaatggscctgtccttgtagtayaaaytartacaccagtcttgtaarccrraraygaaaacytyyttccaaggacaaatcagagaaaaagyctttaactccaccattagcacccaaagctaagattctaatttaaactaytctctgttctttcatggggargcagatttgggtrccacccaagtattgactyayccaycaacaaccgcyatgtatytcgtacattactgcyagycamcatgaatatygyacvgtaccataaayactyrayyacctrtagtacatrmaamyyyaryccryatcaammyyyyvyccyyatgcttacaagcargyacrryaaycraccyycarcyryyayrcatyaryygyarcyccaamryyrcyyctymycyayyagrataycaacarasyyayyyrycytyaacagyacatrgyacatrwwrycatyyrycgtacatagcacattryagtcaaatcyyyyctygycccyayggatgacccccctcagataggrrtcccttgrccaccatcctccgtgaaatcaatatcccgcacaagagtrmtactctcctcgctccgggcccataacacttgggggtagctaaartgaactgtatccgacatctggttcctacttcagggycataaagyctaaatagcccacacgttccccttaaataagacatcacgatg

SEQ ID NO: 2 - DNA, 16569 bp, Homo sapiens
  misc_feature (3106)..(3106): n at 3106 is a deletion
gatcacaggtctatcaccctattaaccactcacgggagctctccatgcatttggtattttcgtctggggggtatgcacgcgatagcattgcgagacgctggagccggagcaccctatgtcgcagtatctgtctttgattcctgcctcatcctattatttatcgcacctacgttcaatattacaggcgaacatacttactaaagtgtgttaattaattaatgcttgtaggacataataataacaattgaatgtctgcacagccactttccacacagacatcataacaaaaaatttccaccaaaccccccctcccccgcttctggccacagcacttaaacacatctctgccaaaccccaaaaacaaagaaccctaacaccagcctaaccagatttcaaattttatcttttggcggtatgcacttttaacagtcaccccccaactaacacattattttcccctcccactcccatactactaatctcatcaatacaacccccgcccatcctacccagcacacacacaccgctgctaaccccataccccgaaccaaccaaaccccaaagacaccccccacagtttatgtagcttacctcctcaaagcaatacactgaaaatgtttagacgggctcacatcaccccataaacaaataggtttggtcctagcctttctattagctcttagtaagattacacatgcaagcatccccgttccagtgagttcaccctctaaatcaccacgatcaaaaggaacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactaagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaaccagagtgtagcttaacacaaagcacccaacttacacttaggagatttcaacttaacttgaccgctctgagctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctancttcaaattcctccctgtacgaaaggacaagagaaataaggcctacttcacaaagcgccttcccccgtaaatgatatcatctcaacttagtattatacccacacccacccaagaacagggtttgttaagatggcagagcccggtaatcgcataaaacttaaaactttacagtcagaggttcaattcctcttcttaacaacatacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttatatgatatgtctccatacccattacaatctccagcattccccctcaaacctaagaaatatgtctgataaaagagttactttgatagagtaaataataggagcttaaacccccttatttctaggactatgagaatcgaacccatccctgagaatccaaaattctccgtgccacctatcacaccccatcctaaagtaaggtcagctaaataagctatcgggcccataccccgaaaatgttggttatacccttcccgtactaattaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgtaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctagacaaaaaaggaaggaatcgaaccccccaaagctggtttcaagccaaccccatggcctccatgactttttcaaaaaggtattagaaaaaccatttcataactttgtcaaagttaaattataggctaaatcctatatatcttaatggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctaccccctctagagcccactgtaaagctaacttagcattaaccttttaagttaaagattaagagaaccaacacctctttacagtgaaatgccccaactaaatactaccgtatggcccaccataattacccccatactccttacactattcctcatcacccaactaaaaatattaaacacaaactaccacctacctccctcaccaaagcccataaaaataaaaaattataacaaaccctgagaaccaaaatgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataatgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatgatggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaattaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttactcttttagtataaatagtaccgttaacttccaattaactagttttgacaacattcaaaaaagagtaataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggcctatgagtgactacaaaaaggattagactgaaccgaattggtatatagtttaaacaaaacgaatgatttcgactcattaaattatgataatcatatttaccaaatgcccctcatttacataaatattatactagcatttaccatctcacttctaggaatactagtatatcgctcacacctcatatcctccctactatgcctagaaggaataatactatcgctgttcattatagctactctcataaccctcaacacccactccctcttagccaatattgtgcctattgccatactagtctttgccgcctgcgaagcagcggtgggcctagccctactagtctcaatctccaacacatatggcctagactacgtacataacctaaacctactccaatgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctcttgtaaatatagtttaaccaaaacatcagattgtgaatctgacaacagaggcttacgaccccttatttaccgagaaagctcacaagaactgctaactcatgcccccatgtctaacaacatggctttctcaacttttaaaggataacagctatccattggtcttaggccccaaaaattttggtgcaactccaaataaaagtaataaccatgcacactactataaccaccctaaccctgacttccctaattccccccatccttaccaccctcgttaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcatcttcctaattaccatactaatcttagttaccgctaacaacctattccaactgttcatcggctgagagggcgtaggaattatatccttcttgctcatcagttgatgatacgcccgagcagatgccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaactactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcaatactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaactaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagatgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaatccacatcaaaaccccctccccatgcttacaagcaagtacagcaatcaaccctcaactatcacacatcaactgcaactccaaagccacccctcacccactaggataccaacaaacctacccacccttaacagtacatagtacataaagccatttaccgtacatagcacattacagtcaaatcccttctcgtccccatggatgacccccctcagataggggtcccttgaccaccatcctccgtgaaatcaatatcccgcacaagagtgctactctcctcgctccgggcccataacacttgggggtagctaaagtgaactgtatccgacatctggttcctacttcagggtcataaagcctaaatagcccacacgttccccttaaataagacatcacgatg

(19) United States
(12) Patent Application Publication
(10) Pub. No.: US 2005/0123913 A1
(43) Pub. Date: Jun. 9, 2005

(54) Human mitochondrial dna polymorphisms, haplogroups, associations with physiological conditions, and genotyping arrays

(75) Inventors: Douglas C. Wallace, Irvine, CA (US); Seyed Hosseini, Duluth, GA (US); Dan Mishmar, Irvine, CA (US); Eduardo Ruiz-Pesini, Irvine, CA (US); Marie Lott, Atlanta, GA (US)

Correspondence Address: Greenlee Winner & Sullivan, Suite 200, 4875 Pearl East Circle, Boulder, CO, 80301, US

(73) Assignee: Emory University, Atlanta, GA (US)

(21) Appl. No.: 10/488,618

(22) PCT Filed: Aug. 30, 2002
(86) PCT No.: PCT/US02/28471

Related U.S. Application Data
(60) Provisional application No. 60/316,333, filed on Aug. 30, 2001.
(60) Provisional application No. 60/380,546, filed on May 13, 2002.

(30) Foreign Application Priority Data
Aug. 31, 2001 (CA) 2356536

(51) Int. Cl. C12Q 1/68; G06F 19/00; G01N 33/48; G01N 33/50

(52) U.S. Cl. 435/6; 702/20

(57) ABSTRACT

This invention provides human mtDNA polymorphisms that are diagnostic of all the major human haplogroups and methods of diagnosing those haplogroups and selected subhaplogroups. This invention also provides methods for identifying evolutionarily significant mitochondrial DNA genes, nucleotide alleles, and amino acid alleles. Evolutionarily significant genes and alleles are identified using one or two populations of a single species. The process of identifying evolutionarily significant nucleotide alleles involves identifying evolutionarily significant genes and then evolutionarily significant nucleotide alleles in those genes, and identifying evolutionarily significant amino acid alleles involves identifying amino acids encoded by all nonsynonymous alleles. Synonymous codings of the nucleotide alleles encoding evolutionarily significant amino acid alleles of this invention are equivalent to the evolutionarily significant amino acid alleles disclosed herein and are included within the scope of this invention. Synonymous codings include alleles at neighboring nucleotide loci that are within the same codon. This invention also provides methods for associating haplogroups and evolutionarily significant nucleotide and amino acid alleles with predispositions to physiological conditions. Methods for diagnosing predisposition to LHON, and methods for diagnosing increased likelihood of developing blindness, centenaria, and increased longevity that are not dependent on the geographical location of the individual being diagnosed are provided herein. Diagnosis of an individual with a predisposition to an energy metabolism-related physiological condition is dependent on the geographic region of the individual. Physiological conditions diagnosable by the methods of this invention include healthy conditions and pathological conditions. Physiological conditions that are associated with haplogroups and with alleles provided by this invention include energetic imbalance, metabolic disease, abnormal energy metabolism, abnormal temperature regulation, abnormal oxidative phosphorylation, abnormal electron transport, obesity, amount of body fat, diabetes, hypertension, and cardiovascular disease.

### CROSS-REFERENCE TO RELATED APPLICATIONS

This application claims priority to U.S. Patent Application Ser. No. 60/316,333 filed Aug. 30, 2001 and Ser. No. 60/380,546 filed May 13, 2002, and to Canadian Patent Application No. 2,356,536 filed on Aug. 31, 2001, which are hereby incorporated in their entirety by reference to the extent not inconsistent with the disclosure herein.

### STATEMENT REGARDING FEDERALLY SPONSORED RESEARCH

This invention was made in part with funding from the United States Government (NIH grants AG13154, HL4017, NS21328, and NS37167). The United States Government may have certain rights therein.

### BACKGROUND OF THE INVENTION

Human mitochondrial DNA (mtDNA) is maternally inherited. Mutations accumulate sequentially in radiating lineages creating branches on the human evolutionary tree. Using sequences of mtDNA, human populations are divisible evolutionarily into haplogroups (Wallace, D. C. et al. (1999) Gene 238:211-230; Ingman M. et al., (2000) Nature 408:708-713; Maca-Meyer, N. (August 2001) BioMed Central 2:13; T. G. Schurr et al., (1999) American Journal of Physical Anthropology 108:1-39; and V. Macaulay et al., (1999) American Journal of Human Genetics 64:232-249). Related haplogroups can be combined into macro-haplogroups. Haplogroups can be subdivided into subhaplogroups. The complete Cambridge mitochondrial DNA sequence may be found at MITOMAP, http://www.gen.emory.edu/cgi-gin/MITOMAP, Genbank accession no. J01415, and is provided in SEQ ID NO:2. Also see Andrews et al. (1999), “Reanalysis and Revision of the Cambridge Reference Sequence for Human Mitochondrial DNA,” Nature Genetics 23:147.

Publications on the subject of mitochondrial biology include: Scheffler, I. E. (1999) Mitochondria, Wiley-Liss, NY; Lestienne P Ed.; Mitochondrial Diseases: Models and Methods, Springer-Verlag, Berlin; Methods in Enzymology (2000) 322: Section V Mitochondria and Apoptosis, Academic Press, CA; Mitochondria and Cell Death (1999) Princeton University Press, NJ; Papa S, Ferruciio G, and Tager J Eds.; Frontiers of Cellular Bioenergetics: Molecular Biology, Biochemistry, and Physiopathology, Kluwer Academic/Plenum Publishers, NY; Lemasters, J. and Nieminen, A. (2001) Mitochondria in Pathogenesis, Kluwer Academic/Plenum Publishers, NY; MITOMAP, http://www.gen.emory.edu/cgi-gin/MITOMAP; Wallace, D. C. (2001) “A mitochondrial paradigm for degenerative diseases and ageing” Novartis Foundation Symposium 235:247-266; Wallace, D. C. (1997) “Mitochondrial DNA in Aging and Disease” Scientific American August 277:40-47; Wallace, D. C. et al., (1998) “Mitochondrial biology, degenerative diseases and aging,” BioFactors 7:187-190; Heddi, A. et al., (1999) “Coordinate Induction of Energy Gene Expression in Tissues of Mitochondrial Disease Patients” JBC 274:22968-22976; Wallace, D. C. (1999) “Mitochondrial Diseases in Man and Mouse” Science 283:1482-1488; Saraste, M. (1999) “Oxidative Phosphorylation at the fin de siecle” Science 283:1488-1493; Kokoszka et. al. (2001) “Increased mitochondrial oxidative stress in the Sod2 (+/−) mouse results in the age-related decline of mitochondrial function culminating in increased apoptosis” PNAS 98:2278-2283; Wallace, D. C. (2001) Mental Retardation and Developmental Disabilities 7:158-166; Wallace, D. C. (2001) Am. J. Med. Gen. 106:71-93; Wei, Y-H et al. (2001) Chinese Medical Journal (Taipei) 64:259-270; and Wallace, D. C. (2001) EuroMit 5 Abstract.

Certain mitochondrial mutations have been associated with physiological conditions (U.S. Pat. No. 6,280,966 issued on Aug. 28, 2001; U.S. Pat. No. 6,140,067 issued on Oct. 31, 2000; U.S. Pat. No. 5,670,320; U.S. Pat. No. 5,296,349; U.S. Pat. No. 5,185,244; U.S. Pat. No. 5,494,794; Wallace, D. C. (1999) Science 283:1482-1488; Brown, M. D. et al. (2001) American Society for Human Genetics Poster #2332; Brown, M. D. et al., (2001) Human Genet. 109:33-39; and Brown, M. D. et al. (January 2002) Human Genet. 110:130-138), Wallace, D. C. et al. (1999) Gene 238:211-230 describes analysis of LHON mutants. Grossman, L. I. et al. (2001) Molecular Phylogenetics and Evolution 18(1):26-36, describes changes in the biochemical machinery for aerobic energy metabolism. Kalman, B. et al. (1999) Acta Neurol. Scand. 99(1): 16-25 describes mitochondrial mutations and multiple sclerosis (MS). Wei, Y. H. et al. (2001) Chinese Medical Journal 64:259-270 describes recent results in support of the mitochondrial theory of aging.

Ivanova, R. et al. (1998) Geronotology 44:349 describes mitochondrial haplotypes and longevity in a French population. Tanaka, M. et al. (1998) Lancet 351:185-186 describes longevity and haplogroups in a Japanese population. De Benedictis, G. et al. (1999) FASEB 13:1532-1536 describes haplogroups and longevity in an Italian population. Rose, G. et al. (2001) European Journal of Human Genetics 9:701-707 describes haplogroup J in centenarians. Ross, O. A. et al. (2001) Experimental Gerontology 36(7):1161-1178 describes haplotypes and longevity in an Irish population.

Haplogroup T has been associated with reduced sperm motility in European males (E. Ruiz-Pesini et al., [2000] American Journal of Human Genetics 67:682-696), the tRNA^{Gln}np 4336 variant in haplogroup H is associated with late-onset Alzheimer Disease (J. M. Shoffner et al., [1993] Genomics 17:171-184).

Taylor, R. W. (1997) J. of Bioenergetics and Biomembranes 29(2):195-205 describes methods for treating mitochondrial disease. Collombet, J. and Coutelle, C. (1998) Molecular Medicine Today 4(1):1-8 describes gene therapy for mitochondrial disorders, including using cell fusion to introduce healthy mitochondria. Owen, R. and Flotte, T. R. (2001) Antioxidants and Redox Signaling 3(3):451-460 discuss approaches and limitations to gene therapy for mitochondrial diseases.

Human mitochondrial DNA sequence variation, except that which has been associated with particular diseases, has not been associated with specific phenotypic conditions, has been considered neutral, and has been used to reconstruct human phylogenies (Henry Gee, “Statistical Cloud over African Eden,” (13 Feb. 1992) Nature 355:583; Marcia Barinaga, “African Eve Backers Beat a Retreat,” (7 Feb. 1992) Science, 255:687; S. Blair Hedges et al., “Human Origins and Analysis of Mitochondrial DNA Sequences,” (7 Feb. 1992) Science, 255:737-739; Allan C. Wilson and Rebecca L. Cann, “The Recent African Genesis of Humans,” (April 1992) Scientific American, 68). The average number of base pair differences between two human mitochondrial genomes is estimated to be from 9.5 to 66 (Zeviani M. et al. (1998) “Reviews in molecular medicine: Mitochondrial disorders,” Medicine 77:59-72).

The D-loop is the most variable region in the mitochondrial genome, and the most polymorphic nucleotide sites within this loop are concentrated in two ‘hypervariable segments’, HVS-I and HVS-II (Wilkinson-Herbots, H. M. et al., (1996) “Site 73 in hypervariable region II of the human mitochondrial genome and the origin of European populations,” Ann Hum Genet 60:499-508). Population-specific, neutral mtDNA variants have been identified by surveying mtDNA restriction site variants or by sequencing hypervariable segments in the displacement loop. Restriction analysis using fourteen restriction endonucleases allowed screening of 15-20% of the mtDNA sequence for variations (Chen Y. S. et al., (1995) “Analysis of mtDNA variation in African populations reveals the most ancient of all human continent-specific haplogroups,” Am J Hum Genet 57:133-149). The large majority of mtDNA sequence data published to date are limited to HVS-I. Bandelt, H. J. et al., (1995) “Mitochondrial portraits of human populations using median networks” Genetics 141:743-753).

The coding and classification system that has been used for mtDNA haplogroups refers primarily to the information provided by RFLPs and the hypervariable segments of the control region. (Torroni, A. et al. (1996) “Classification of European mtDNAs from an analysis of three European populations,” Genetics 144:1835-1850 and Richards M B et al., (1998) “Phylogeography of mitochondrial DNA in western Europe,” Ann Hum Genet 62:241-260.)

Methods are known for testing the likelihood of neutrality of mutations (Tajima, F. (1989) Genetics 123:585-595; Fu, Y. and Li, W. (1993) Genetics 133:693-709; Li, W. et al. (1985) Mol. Biol. Evol. 2(2):150-174; and Nei, M. and Gojobori, T. (198.6) Mol. Biol. Evol. 3(5):418-426). All of the methods in these publications are used to compare datasets taken from separate groups. None of these methods are used to analyze a dataset not containing data representing an outgroup.

Wise, C. A. et al. (1998) Genetics 148:409-421, describes neutrality analysis of the human mitochondrial NADH Dehydrogenase Subunit 2 gene, when compared to the NADH Dehydrogenase Subunit 2 gene from chimpanzees. Templeton, A. R. (1996) Genetics 144:1263-1270, describes neutrality analysis of the human mitochondrial Cytochrome Oxidase II (COXII) gene when compared to the COXII gene in hominoid primates. Messier, W. and Stewart, C. (1997) Nature 385:151-154 describes neutrality analysis of primate lysozymes. Endo, T. et al. (1996) Mol. Biol. Evol. 13(5):685-690 describes large-scale neutrality analysis of sequences from DDBJ, EMBL, and GenBank databases. Hughes, A. L. and Nei, M. (1988) Nature 335:167-170 describes neutrality analysis of MC Class I loci. Nachman, M. W. (1996) Genetics 142:953-963 describes neutrality analysis of the human mitochondrial NADH Dehydrogenase subunit 3 (NADH3) gene, when compared to the NADH Dehydrogenase subunit 3 gene from chimpanzees. Nachman, M. W. et al. (1994) Proc. Nat. Acad. Sci. USA 76:5269-5273 describes neutrality analysis of the mitochondrial NADH dehydrogenase subunit 3 gene in 3 strains of mouse. Rand, D. M. et al. (1994) Genetics 138:741-756; Ballard, J. W. O. and Kreitman, M. (1994) Genetics 138:757-772; and Kaneko, M. Y. et al. (1993) Genet. Res. 61:195-204, describe neutrality analysis for mitochondrial NADH dehydrogenase subunit 5, Cytochrome b, and ATPase6 in strains of Drosophila.

In the above-mentioned publications, neutrality testing, including Kₐ/Kₛ analysis, has not been applied for the purpose of identifying disease-associated mutations. Populations for neutrality testing analysis were identified by observation of normal phenotypic variation. Neutrality testing has been performed to determine whether a gene is under selection. None of these publications describe neutrality analysis with the purpose of identifying phenotype-associated mutations, and no suspected phenotype-associated mutations were identified.

U.S. Pat. No. 6,228,586 (issued May 8, 2001) and U.S. Pat. No. 6,280,953 (issued Aug. 28, 2001) describe methods for identifying polynucleotide and polypeptide sequences in human and/or non-human primates, which may be associated with a physiological condition. The methods employ comparison of human and non-human primate sequences using statistical methods. U.S. Pat. No. 6,274,319 (issued Aug. 14, 2001) describes Kₐ/Kₛ methods for identifying polynucleotide and polypeptide sequences that may be associated with commercially or aesthetically relevant traits in domesticated plants or animals. The methods employ comparison of homologous genes from the domesticated organism and its wild ancestor to identify evolutionarily significant changes. In the above-mentioned publications, neutrality testing, including Kₐ/Kₛ analysis, is only applied to interspecific, not intraspecific, comparisons, and only genes from the nuclear genome, not from organelle genomes, are analyzed.

Methods for constructing peptide and nucleotide libraries are well known to the art, e.g. as described in U.S. Pat. Nos. 6,156,511 and 6,130,092. Sequencing methods are also known to the art, e.g., as described in U.S. Pat. No. 6,087,095. Arrays of nucleic acid have been used for sequencing and for identifying exceptional alleles including disease-associated alleles. Nucleic acid arrays have been described, e.g., in patent nos.: U.S. Pat. Nos. 5,837,832, 5,807,522, 6,007,987, 6,110,426, WO 99/05324, 99/05591, WO 00/58516, WO 95/11995, WO 95/35505A1, WO 99/42813, JP10503841T2, GR3030430T3, ES2134481T3, EP804731B1, DE69509925C0, CA2192095AA, AU2862995A1, AU709276B2, AT180570, EP 1066506, and AU 2780499. Computational methods are useful for analyzing hybridization results, e.g., as described in PCT Publication WO 99/05574, and U.S. Pat. Nos. 5,754,524; 6228,575; 5,593,839; and 5,856,101. Methods for screening for disease markers are also known to the art, e.g. as described in U.S. Pat. Nos. 6,228,586; 6,160,104; 6,083,698; 6,268,398; 6,228,578; and 6,265,174.

The development of microarray technologies has stemmed from the desire to examine very large numbers of nucleic acid probe sequences simultaneously, in an effort to obtain information about genetic mutations, gene expression or nucleic acid sequences. Microarray technologies are intimately connected with the Human Genome Project, which has development of rapid methods of nucleic acid sequencing and genome analysis as key objectives (E. Marshall, (1995) Science 268:1270), as well as elucidation of sequence-function relationships (M. Schena et al., (1996) Proc. Nat'l. Acad. Sci. USA, 93:10614). Microarray hybridization of PCR-amplified fragments to allele-specific oligonucleotide (ASO) probes is widely used in large-scale single nucleotide polymorphism (SNP) genotyping (Huber M. et al. (2002) Analytical Biochemistry 303:25-33 and Southern, E. M. (1996) Trends Genet. 12:110-115).

The Affymetrix GeneChip® HuSNP™ Array enables whole-genome surveys by simultaneously tracking nearly 1,500 genetic variations, known as single nucleotide polymorphisms (SNPs), dispersed throughout the genome. The HuSNP Affymetrix Array is being used for familial linkage studies that aim to map inherited disease or drug susceptibilities as well as for tracking de novo genetic alterations. For genotyping, arrays rely on multiple probes to interrogate individual nucleotides in a sequence. The identity of a target base can be deduced using four identical probes that vary only in the target position, each containing one of the four possible bases. Alternatively, the presence of a consensus sequence can be tested using one or two probes representing specific alleles. To genotype heterozygous or genetically mixed samples, arrays with many probes can be created to provide redundant information.

Arrays, also called DNA microarrays or DNA chips, are fabricated by high-speed robotics, generally on glass but sometimes on nylon substrates, for which probes (Phimister, B. (1999) Nature Genetics 21 s: 1-60) with known identity are used to determine complementary binding. An experiment with a single DNA chip can provide researchers information on thousands of genes simultaneously. There are several steps in the design and implementation of a DNA array experiment. Many strategies have been investigated at each of these steps: 1) DNA types; 2) Chip fabrication; 3) Sample preparation; 4) Assay; 5) Readout; and 6) Software (informatics).

There are two major application forms for the array technology: 1) Determination of expression level (abundance) of genes; and 2) Identification of sequence (gene/gene mutation). There appear to be two variants of the array technology, in terms of intellectual property, of arrayed DNA sequence with known identity: Format I consists of probe cDNA (500˜5,000 bases long) immobilized to a solid surface such as glass using robot spotting and exposed to a set of targets either separately or in a mixture. This method, “traditionally” called DNA microarray, is widely considered as having been developed at Stanford University. (R. Ekins and F. W. Chu “Microarrays: their origins and applications,” [1999] Trends in Biotechnology, 17:217-218). Format II consists of an array of oligonucleotide (20˜80-mer oligos) or peptide nucleic acid (PNA) probes synthesized either in situ (on-chip) or by conventional synthesis followed by on-chip immobilization. The array is exposed to labeled sample DNA, hybridized, and the identity/abundance of complementary sequences is determined. This method, “historically” called DNA chips, was developed at Affymetrix, Inc., which sells its photolithographically fabricated products under the GeneChip® trademark. Many companies are manufacturing oligonucleotide-based chips using alternative in-situ synthesis or depositioning technologies.

Probes on arrays can be hybridized with fluorescently-labeled target polynucleotides and the hybridized array can be scanned by means of scanning fluorescence microscopy. The fluorescence patterns are then analyzed by an algorithm that determines the extent of mismatch content identifies polymorphisms, and provides some general sequencing information (M. Chee et al., [1996] Science 274:610). Selectivity is afforded in this system by low stringency washes to rinse away non-selectively adsorbed materials. Subsequent analysis of relative binding signals from array elements determines where base-pair mismatches may exist. This method then relies on conventional chemical methods to maximize stringency, and automated pattern recognition processing is used to discriminate between fully complementary and partially complementary binding.

Devices such as standard nucleic acid microarrays or gene chips, require data processing algorithms and the use of sample redundancy (i.e., many of the same types of array elements for statistically significant data interpretation and avoidance of anomalies) to provide semi-quantitative analysis of polymorphisms or levels of mismatch between the target sequence and sequences immobilized on the device surface.

Labels appropriate for array analysis are known in the art. Examples are the two-color fluorescent systems, such as Cy3/Cy5 and Cy3.5/Cy5.5 phosphoramidites (Glen Research, Sterling Va.). Patents covering cyanine dyes include: U.S. Pat. No. 6,114,350 (Sep. 5, 2000); U.S. Pat. No. 6,197,956 (Mar. 6, 2001); U.S. Pat. No. 6,204,389 (Mar. 20, 2001) and U.S. Pat. No. 6,224,644 (May 1, 2001). Array printers and readers are available in the art.

A process of using arrays is described in Grigorenko, E. V. ed., (2002) DNA Arrays: Technologies and Experimental Strategies, CRC Press, NY; Vrana, K. E. et al., (May 2001) Microarrays and Related Technologies: Miniaturization and Acceleration of Genomics Research, CHI, Upper Falls, Mass.; and Branca, M. A. et al., (February 2002) DNA Microarray Informatics: Key Technological Trends and Commercial Opportunities, CHI, Upper Falls, Mass.

All publications referred to herein are incorporated by reference to the extent not inconsistent herewith. The mention of a publication in this Background Section does not constitute an admission that it is prior art.

### SUMMARY OF INVENTION

The high mitochondrial DNA mutation rate of human mitochondrial DNA has been thought to result in the accumulation of a wide range of neutral, population-specific base substitutions in mtDNA. These have accumulated sequentially along radiating maternal lineages that have diverged approximately on the same time scale as human populations have colonized different geographical regions of the world.

About 76% of all African mtDNAs fall into haplogroup L, defined by an HpaI restriction site gain at bp 3592.77% of Asian mtDNAs are encompassed within a super-haplogroup defined by a DdeI site gain at bp 10394 and an AluI site gain at bp 10397. Essentially all native American mtDNAs fall into four haplogroups, A-D. Haplogroup A is defined by a HaeIII site gain at bp 663, B by a 9 bp deletion between bp 8271 to bp 8281, C by a HincII site loss at bp 13259, and D defined by an AluI site loss at bp 5176. Ten haplogroups encompass almost all mtDNAs in European populations. The ten-mtDNA haplogroups of Europeans can be surveyed by using a combination of data from RFLP analysis of the coding region and sequencing of the hypervariable segment I. About 99% of European mtDNAs fall into one of ten haplogroups: H, I, J, K, M, T, U, V, W or X.

This invention provides human mtDNA polymorphisms that are diagnostic of all the major human haplogroups and methods of diagnosing those haplogroups and selected sub-haplogroups.

This invention also provides methods for identifying evolutionarily significant mitochondrial DNA genes, nucleotide alleles, and amino acid alleles. Evolutionarily significant genes and alleles are identified using one or two populations of a single species. The process of identifying evolutionarily significant nucleotide alleles involves identifying evolutionarily significant genes and then evolutionarily significant nucleotide alleles in those genes, and identifying evolutionarily significant amino acid alleles involves identifying amino acids encoded by all nonsynonymous alleles. Synonymous codings of the nucleotide alleles encoding evolutionarily significant amino acid alleles of this invention are equivalent to the evolutionarily significant amino acid alleles disclosed herein and are included within the scope of this invention. Synonymous codings include alleles at neighboring nucleotide loci that are within the same codon.

This invention also provides methods for associating haplogroups and evolutionarily significant nucleotide and amino acid alleles with predispositions to physiological conditions. Methods for diagnosing predisposition to LHON, and methods for diagnosing increased likelihood of developing blindness, centenaria, and increased longevity that are not dependent on the geographical location of the individual being diagnosed are provided herein. Diagnosis of an individual with a predisposition to an energy metabolism-related physiological condition is dependent on the geographic region of the individual. Physiological conditions diagnosable by the methods of this invention include healthy conditions and pathological conditions. Physiological conditions that are associated with haplogroups and with alleles provided by this invention include energetic imbalance, metabolic disease, abnormal energy metabolism, abnormal temperature regulation, abnormal oxidative phosphorylation, abnormal electron transport, obesity, amount of body fat, diabetes, hypertension, and cardiovascular disease.

Molecules having sequences provided by this invention are provided in libraries and on genotyping arrays. This invention provides methods of making and using the genotyping arrays of this invention. The arrays of this invention are useful for determining the presence and absence of nucleotide alleles of this invention, for determining a haplogroup, and for diagnosis.

This invention also provides machine-readable storage devices and program devices for storing data and programmed methods for diagnosing haplogroups and physiological conditions.

The arrays of this invention are useful for determining the presence and absence of nucleotide alleles of this invention, for determining a haplogroup, and for diagnosis. This invention also provides machine-readable storage devices and program devices for storing data and programmed methods for diagnosing haplogroups and physiological conditions.

### BRIEF DESCRIPTION OF THE FIGURES

FIG. 1 shows a consensus neighbor-joining tree of 104 human mtDNA complete sequences and two primate sequences. Numbers correspond to bootstrap values (% of 500 total bootstrap replicates) (Felsenstein, J. (1993) PHYLIP (Phylogeny Inference Package) 3.53c. Distributed by author, Department of Genetics, University of Washington, Seattle, Wash.). Maximum Likelihood (ML) and UPGMA yielded consistent branching orders with respect to continent-specific mtDNA haplogroups. Sequences: 11-53: Genbank AF346963-AF347015 (4); E21U: Genbank X93334, A1L1a: Genbank D38112, cam revise: Genbank NC_{—}001807 corrected according to (R. M. Andrews et al., Nature Genetics 23, 147 (1999)); the rest are 48 sequences generated in this invention using an ABI 377. Specific mutations in patient samples that have been implicated in disease were excluded from this analysis, as well as gaps and deletions, with the exception of the 9 bp deletion (nucleotide position (np) 8272 to 8281). Haplogroups A, B, C, D, and X were drawn from both Eurasia and the Americas. Haplogroup names are designated with capital letters. P. paniscus and P. troglodytes mtDNA sequences were used as outgroups. Haplogroups L0 and L1 encompass previously assigned L1a and L1b mtDNAs, respectively (Y. S. Chen et al., American Journal of Human Genetics 66, 1362-1383 (2000)).

FIG. 2 shows the migrations of human haplogroups around the world. +/−, +/+, or −/− equals Dde I 10394 and Alu I 10397. * equals Rsa I 16329. The mutation rate is 2.2-2.9% per million years. Time estimates are YBP (years before present).

FIG. 3 shows a cladogram listing nucleotide alleles describing 21 major human haplogroups, 21 sub-haplogroups, and several macro-haplogroups. The groups on the left are described by the alleles to their right. A vertical bar designates that each group to the left of the bar has all of the alleles to the right of the bar.

FIG. 4 shows the selective constraint (k_{C} values) of mtDNA protein genes with comparisons among mammalian species. Statistical significance (P<0.05) was determined using ANOVA, t-tests or the Tukey-Kramer Multiple Comparisons tests. Most programs used are from DNAsp (J. Rozas and R. Rozas, (1999) Bioinformatics 15:174-5). DNA sequence divergence was analyzed using the DIVERGE program (Wisconsin Package Version 10.0, Genetics Computer Group (GCG), Madison, Wis.). For all thirteen mtDNA genes, data is shown for human, human compared to P. troglodytes, human compared to P. paniscus, and nine species of primates. For only ATP6 and ATP8, data is also shown for fourteen species of mammals.

### DETAILED DESCRIPTION OF THE INVENTION

Table 1 shows human mitochondrial nucleotide alleles, which have been associated with physiological conditions. In Table 1, columns three (nucleotide locus), five (physiological condition nucleotide allele), and column two (physiological condition) make up the set of Human Mitochondrial Nucleotide Alleles Known to be Associated with Physiological Conditions.

TABLE 1¹
Human Mitochondrial Alleles Known to be Associated with Physiological Conditions

| | | | | Physiological | | Physiological |
|---|---|---|---|---|---|---|
| | | | Cambridge | Condition | Cambridge | Condition |
| | | Nucleotide | Nucleotide | Nucleotide | Amino Acid | Amino Acid |
| Gene | Physiological Condition | Locus | Allele | Allele | Allele | Allele |
| MTND1 | *MELAS | 3308 | T | C | M | T |
| MTND1 | *NIDDM; LHON; PEO | 3316 | G | A | A | T |
| MTND1 | *LHON | 3394 | T | C | Y | H |
| MTND1 | *NIDDM | 3394 | T | C | Y | H |
| MTND1 | *ADPD | 3397 | A | G | M | V |
| MTND1 | *LHON | 3460 | G | A | A | T |
| MTND1 | *LHON | 3496 | G | T | A | S |
| MTND1 | *LHON | 3497 | C | T | A | V |
| MTND1 | *LHON | 4136 | A | G | Y | C |
| MTND1 | *LHON | 4160 | T | C | L | P |
| MTND1 | *LHON | 4216 | T | C | Y | H |
| MTND2 | *LHON | 4917 | A | G | D | N |
| MTND2 | *LHON | 5244 | G | A | G | S |
| MTND2 | *AD | 5460 | G | A | A | T |
| MTND2 | *AD | 5460 | G | T | A | S |
| MTCO1 | *Myoglobinuria, Exercise Intolerance | 5920 | G | A | W | Ter |
| MTCO1 | *Multisystem Disorder | 6930 | G | A | G | Ter |
| MTCO1 | *LHON | 7444 | G | A | Ter | K |
| MTCO2 | *Mitochondrial Encephalomyopathy | 7587 | T | C | M | T |
| MTCO2 | *MM | 7671 | T | A | M | K |
| MTCO2 | *Multisystem Disorder | 7896 | G | A | W | Ter |
| MTCO2 | *Lactic Acidosis | 8042 | AT | 2 nt del (AT) | M | Ter |
| MTATP6 | *NARP | 8993 | T | G | L | R |
| MTATP6 | *NARP/Leigh Disease | 8993 | T | C | L | P |
| MTATP6 | *LHON | 9101 | T | C | I | T |
| MTATP6 | *FBSN/Leigh Disease | 9176 | T | C | L | P |
| MTATP6 | *Leigh Disease | 9176 | T | G | L | R |
| MTCO3 | *LHON | 9438 | G | A | G | S |
| MTCO3 | *Leigh-like | 9537 | C | C ins | Q | frameshift |
| MTCO3 | *LHON | 9738 | G | T | A | S |
| MTCO3 | *LHON | 9804 | G | A | A | T |
| MTCO3 | *Mitochondrial Encephalopathy | 9952 | G | A | W | Ter |
| MTCO3 | *PEM; MELAS | 9957 | T | C | F | L |
| MTND3 | *ESOC | 10191 | T | C | S | P |
| MTND4 | *MELAS | 11084 | A | G | T | A |
| MTND4 | *LHON | 11778 | G | A | R | H |
| MTND4 | *Exercise Intolerance | 11832 | G | A | W | Ter |
| MTND4 | *DM | 12026 | A | G | I | V |
| MTND5 | *MELAS | 13513 | G | A | D | N |
| MTND5 | *MELAS | 13514 | A | G | D | G |
| MTND5 | *LHON-like | 13528 | A | G | T | A |
| MTND5 | *LHON | 13708 | G | A | A | T |
| MTND5 | *LHON | 13730 | G | A | G | E |
| MTND6 | *MELAS | 14453 | G | A | A | V |
| MTND6 | *LDYT | 14459 | G | A | A | V |
| MTND6 | *LHON | 14484 | T | C | M | V |
| MTND6 | *LHON | 14495 | A | G | L | S |
| MTND6 | *LHON | 14568 | C | T | G | S |
| MTCYB | *PD/MELAS | 14787 | TTAA | 4 nt del | I | frameshift |
| | | | | (TTAA) | | |
| MTCYB | *MM | 15059 | G | A | G | Ter |
| MTCYB | *Exercise Intolerance | 15150 | G | A | W | Ter |
| MTCYB | *Exercise Intolerance | 15197 | T | C | S | P |
| MTCYB | *Mitochondrial Encephalomyopathy | 15242 | G | A | G | Ter |
| MTCYB | *LHON | 15257 | G | A | D | N |
| MTCYB | *Exercise Intolerance | 15615 | G | A | G | D |
| MTCYB | *MM | 15762 | G | A | G | E |
| MTCYB | *LHON | 15812 | G | A | V | M |

¹(MITOMAP: A Human Mitochondrial Genome Database. Center for Molecular Medicine, Emory University, Atlanta, GA, USA. http://www.gen.emory.edu/mitomap.html, 2001).
*Definitions:
LHON Leber Hereditary Optic Neuropathy
MM Mitochondrial Myopathy
AD Alzheimer's Disease
LIMM Lethal Infantile Mitochondrial Myopathy
ADPD Alzheimer's Disease and Parkinson's Disease
MMC Maternal Myopathy and Cardiomyopathy
NARP Neurogenic muscle weakness, Ataxia, and Retinitis Pigmentosa; alternate phenotype at this locus is reported as Leigh Disease
FICP Fatal Infantile Cardiomyopathy Plus a MELAS-associated Cardiomyopathy
MELAS Mitochondrial Encephalomyopathy, Lactic Acidosis, and Stroke-like episodes LDYT Leber's hereditary optic neuropathy and DYsTonia
MERRF Myoclonic Epilepsy and Ragged Red Muscle Fibers
MHCM Maternally inherited Hypertrophic CardioMyopathy
CPEO Chronic Progressive External Ophthalmoplegia
KSS Kearns Sayre Syndrome
DM Diabetes Mellitus
DMDF Diabetes Mellitus + DeaFness
CIPO Chronic Intestinal Pseudoobstructton with myopathy and Ophthalmoplegia
DEAF Maternally inherited DEAFness or aminoglycoside-induced DEAFness
PEM Progressive encephalopathy
SNHL SensoriNeural Hearing Loss

Thirteen protein-coding mitochondrial genes are known (MitoMap, http://www.gen.emory.edu/cgi-bin/MITOMAP).

TABLE 2
Protein-coding Human MtDNA Genes

| Gene | Map Locus^{a} | Abbreviation | Location^{b} |
|---|---|---|---|
| NADH dehydrogenase 1 | MTND1 | ND1 | 3307-4262 |
| NADH dehydrogenase 2 | MTND2 | ND2 | 4470-5511 |
| NADH dehydrogenase 3 | MTND3 | ND3 | 10059-10404 |
| NADH dehydrogenase 4L | MTND4L | ND4L | 10470-10766 |
| NADH dehydrogenase 4 | MTND4 | ND4 | 10760-12137 |
| NADH dehydrogenase 5 | MTND5 | ND5 | 12337-14148 |
| NADH dehydrogenase 6 | MTND6 | ND6 | 14149-14673 |
| Cytochrome b | MTCYB | Cytb | 14747-15887 |
| Cytochrome c oxidase I | MTCO1 | COI | 5904-7445 |
| Cytochrome c oxidase II | MTCO2 | COII | 7586-8269 |
| Cytochrome c oxidase III | MTCO3 | COIII | 9207-9990 |
| ATP synthase 6 | MTATP6 | ATP6 | 8527-9207 |
| ATP synthase 8 | MTATP8 | ATP8 | 8366-8572 |

^{a,b}As defined on MitoMap, http://www.gen.emory.edu/cgi-bin/MITOMAP, which is numbered relative to the Cambridge Sequence (Genbank accession no. J01415 and Andrews et al. (1999), A Reanalysis and Revision of the Cambridge Reference Sequence for Human Mitochondrial DNA, Nature Genetics 23: 147.

Codon usage for mtDNA differs slightly from the universal code. For example, UGA codes for typtophan instead of termination, AUA codes for methionine instead of isoleucine, and AGA and AGG are terminators instead of coding for arginine.

As used herein “printing” refers to the process of creating an array of nucleic acids on known positions of a solid substrate. The arrays of this invention can be printed by spotting, e.g., applying arrays of probes to a solid substrate, or to the synthesis of probes in place on a solid substrate. As used herein “glass slide” refers to a small piece of glass of the same dimensions as a standard microscope slide. As used herein, “prepared substrate” refers to a substrate that is prepared with a substance capable of serving as an attachment medium for attaching the probes to the substrate, such as poly Lysine. As used herein, “sample” refers to a composition containing human mitochondrial DNA that can be genotyped. As used herein, “quantitative hybridization” refers to hybridization performed under appropriate conditions and using appropriate materials such that the sequence of one nucleotide allele (a single nucleotide polymorphism) can be determined, such as by hybridization of a molecule containing that allele to two or more probes, each containing different alleles at that nucleotide locus, all as is known in the art.

As used herein, “physiological condition” includes diseased conditions, healthy conditions, and cosmetic conditions. Diseased conditions include, but are not limited to, metabolic diseases such as diabetes, hypertension, and cardiovascular disease. Healthy conditions include, but are not limited to, traits such as increased longevity. Physiological conditions include cosmetic conditions. Cosmetic conditions include, but are not limited to, traits such as amount of body fat. Physiological conditions can change health status in different contexts, such as for the same organism in a different environment. Such different environments for humans are different cultural environments or different climatic contexts such as are found on different continents.

As used herein, “neutrality analysis” refers to analysis to determine the neutrality of one or more nucleotide alleles and/or the gene containing the allele(s) using at least two alleles of a sequence. Commonly, the alleles in a sequence to be analyzed are divided into two groups, synonymous and nonsynonymous. Codon usage tables showing which codons encode which amino acids are used in this analysis. Codon usage tables for many organisms and genomes are available in the art. If a gene is determined to not be neutral, the gene is determined to have had selection pressure applied to it during evolution, and to be evolutionarily significant. The alleles that change amino acids in the gene (nonsynonymous) are then determined to be non-neutral and evolutionarily significant.

As used herein, “Kₐ/Kₛ” refers to a ratio of the proportion of nonsynonymous differences to the proportion of synonymous differences in a DNA sequence analysis, as is known to the art. The proportion of nonsynonymous differences is the number of nonsynonymous nucleotide substitutions in a sequence per site at which a nonsynonymous substitution could occur. The proportion of synonymous differences is the number of synonymous nucleotide substitutions in a sequence per site at which synonymous substitutions could occur. Alternatively, instead of only including the number of sites in the denominator of each proportion, the number of alternative substitutions that could occur at each site are also included. Either definition may be used as long as similar definitions are used for both Kₐ and Kₛ in an analysis. K_{C} is Kₐ/Kₛ.

As used herein “nonsynonymous” refers to mutations that result in changes to the encoded amino acid. As used herein, “synonymous” refers to mutations that do not result in changes to the encoded amino acids.

As used herein, “haplogroup” refers to radiating lineages on the human evolutionary tree, as is known in the art. As used herein, “macro-haplogroup” refers to a group of evolutionarily related haplogroups. As used herein, “sub-haplogroup” refers to an evolutionarily related subset of a haplogroup. An individual's haplotype is the haplogroup to which he belongs.

As used herein, “extended longevity” or “extended lifespan” refers to living longer than the average expected lifespan for the population to which one belongs. As used herein, “centenaria” refers to an extended lifespan that is at least 100 years.

As used herein, “abnormal energy metabolism” in an individual who is non-native to the geographical region in which he lives refers to energy metabolism that differs from that of the population that is native to where the individual lives. As used herein, “abnormal temperature regulation” in such an individual refers to temperature regulation that differs from that of the population that is native to where he lives. As used herein, “abnormal oxidative phosphorylation” in such an individual refers to oxidative phosphorylation that differs from that of the population that is native to where he lives. As used herein, “abnormal electron transport” in such an individual refers to electron transport that differs from that of the population that is native to where he lives. As used herein “metabolic disease” of such an individual refers to metabolism that differs from that of the population that is native to where he lives. As used herein, “energetic imbalance” of such an individual refers to a balance of energy generation or use that differs from that of the population that is native to where he lives. As used herein, “obesity” of such an individual refers to a body weight that, for the height of the individual, is 20% higher than the average body weight that is recommended for the population native to where the individual lives. As used herein, “amount of body fat” of such an individual refers to a low or high percentage of body fat relative to what is recommended for the population that is native to where he lives.

As used herein, an isolated nucleic acid is a nucleic acid outside of the context in which it is found in nature. The term covers, for example: (a) a DNA which has the sequence of part of a naturally-occurring genomic DNA molecule but is not flanked by both of the coding or noncoding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally-occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein, or a modified gene having a sequence not found in nature.

As used herein, “nucleotide locus” refers to a nucleotide position of the human mitochondrial genome. The Cambridge sequence SEQ ID NO:2 is used as a reference sequence, and the positions of the mitochondrial genome referred to herein are assigned relative to that sequence. As used herein, “loci” refers to more than one locus. As used herein, “nucleotide allele” refers to a single nucleotide at a selected nucleotide locus from a selected sequence when different bases occur naturally at that locus in different individuals. The nucleotide allele information is provided herein as the nucleotide locus number and the base that is at that locus, such as 3796C, which means that at human mitochondrial position 3796 in the Cambridge sequence, there is a cytosine (C). As used herein, “amino acid allele” refers to the amino acid that is at a selected amino acid location in the human mitochondrial genome when different amino acids occur naturally at that location in different individuals. There are thirteen protein-coding genes in the human mitochondria. For each gene, the encoded protein consists of amino acids that are numbered starting at one. ND1 304H, means that there is a histidine at amino acid 304 in the ND1 protein. Amino acids are encoded by codons. As used herein, “codon” refers to the group of three nucleotides that encode an amino acid in a protein, as is known in the art. An amino acid allele can be referred to by one or more of the nucleotide loci that code for it. For example, ntl 15884 P means that there is a proline (P) encoded by the codon containing nucleotide locus 15884.

As used herein, “evolutionarily significant gene” refers to a gene that has statistically significantly more nonsynonymous nucleotide changes, when compared to the corresponding gene in another individual, than would be expected by chance. As used herein, “evolutionarily significant nucleotide allele” refers to a nucleotide allele that is located in a gene that has been determined to be evolutionarily significant using that nucleotide allele, or an equivalent nucleotide allele in a corresponding gene in another individual. As used herein, “intraspecific” means within one species. As used herein, “subpopulation” refers to a population within a larger population. A subpopulation can be as small as one individual. As used herein, “geographic region” refers to a geographic area in which a statistically significant number of individuals have the same haplotype. As used herein, being “native” to a geographic region refers to having the haplotype associated with that geographic region. The haplotype associated with a geographic region is that which originated in the region or of many individuals who settled historically in the region with respect to human evolution.

As used herein, “target” or “target sample” refers to the collection of nucleic acids used as a sample for array analysis. The target is interrogated by the probes of the array. A “target” or “target sample” may be a mixture of several samples that are combined. For example, an experimental target sample may be combined with a differently labeled control target sample and hybridized to an array, the combined samples being referred to as the “target” interrogated by the probes of the array during that experiment. As used herein, “interrogated” means tested. Probes, targets, and hybridization conditions are chosen such that the probes are capable of interrogating the target, i.e., of hybridizing to complementary sequences in the target sample.

As used herein, “increased likelihood of developing blindness” refers to a higher than normal probability of losing the ability to see normally and/or of losing the ability to see normally at a younger age.

All sequences defined herein are meant to encompass the complementary strand as well as double-stranded polynucleotides comprising the given sequence.

This invention provides a list of human mtDNA polymorphisms found in all the major human haplogroups. Example 1 summarizes data from sequencing over 100 human mtDNA genomes that are representative of the major human haplogroups around the world. The summary includes over 900 point mutations and one nine-base pair deletion. Table 3, Human MtDNA Nucleotide Alleles, lists the alleles identified in 103 such sequences in the third column, the corresponding alleles of the Cambridge mtDNA sequence in the second column and the nucleotide loci (position in the Cambridge sequence), in the first column. Table 3 lists the set of human mtDNA nucleotide alleles that occur naturally in different haplogroups. Table 3 does not include alleles previously known to be associated with disease (i.e., does not include the alleles of Table 1). The nucleotide alleles listed in column three of Table 3, together with the corresponding nucleotide loci in column one, make up the set of non-Cambridge human mtDNA nucleotide alleles. Table 4 lists the nucleotide alleles identified by the inventors hereof in 48 human mtDNA genomes in column three, and the corresponding Cambridge alleles in column two. Columns one and three of Table 4 make up the set of non-Cambridge human mtDNA nucleotide alleles in 48 genomes.

The nucleotide alleles listed in Table 3, including the Cambridge nucleotide alleles, being naturally occurring, are useful for identifying alleles that are associated with abnormal physiological conditions. These nucleotide alleles can be ignored during analysis steps when performing methods for identifying novel alleles associated with selected physiological conditions.

As described below, certain alleles of Table 3 are useful for identifying physiological conditions related to energy metabolism such as energetic imbalance, metabolic disease, abnormal energy metabolism, abnormal temperature regulation, abnormal oxidative phosphorylation, abnormal electron transport, obesity, amount of body fat, diabetes, hypertension, and cardiovascular disease when the affected individuals have the abnormal physiological condition because they are in a geographical region that is not native for their haplogroup.

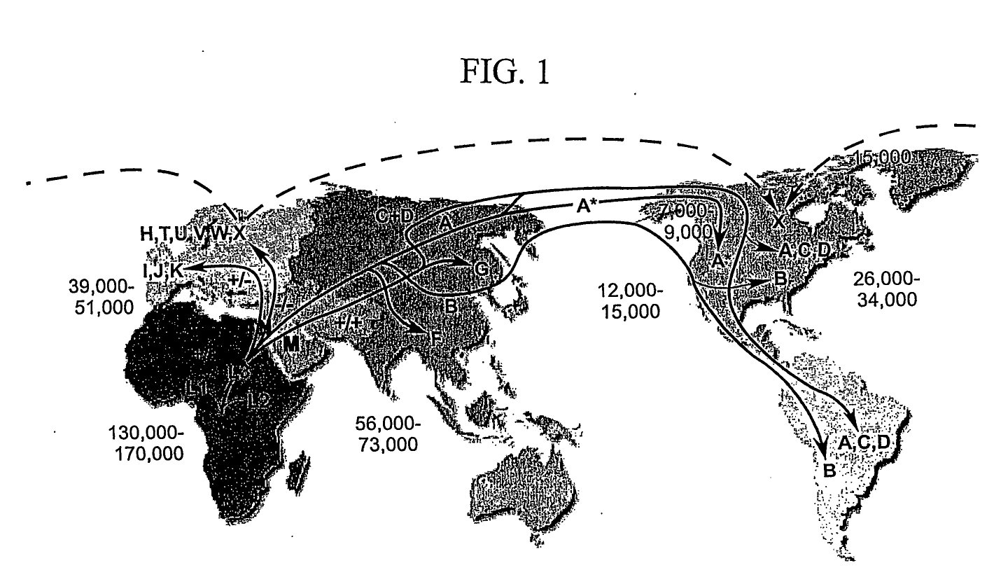

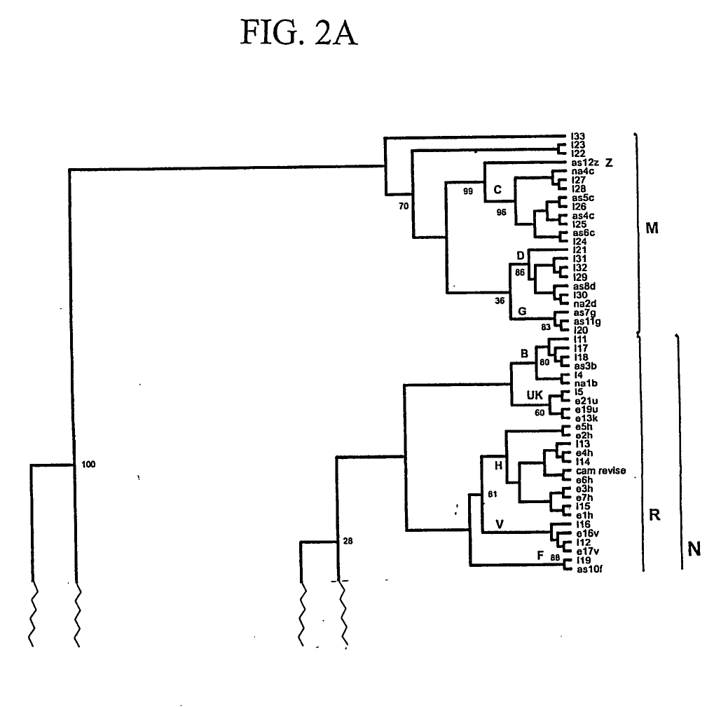

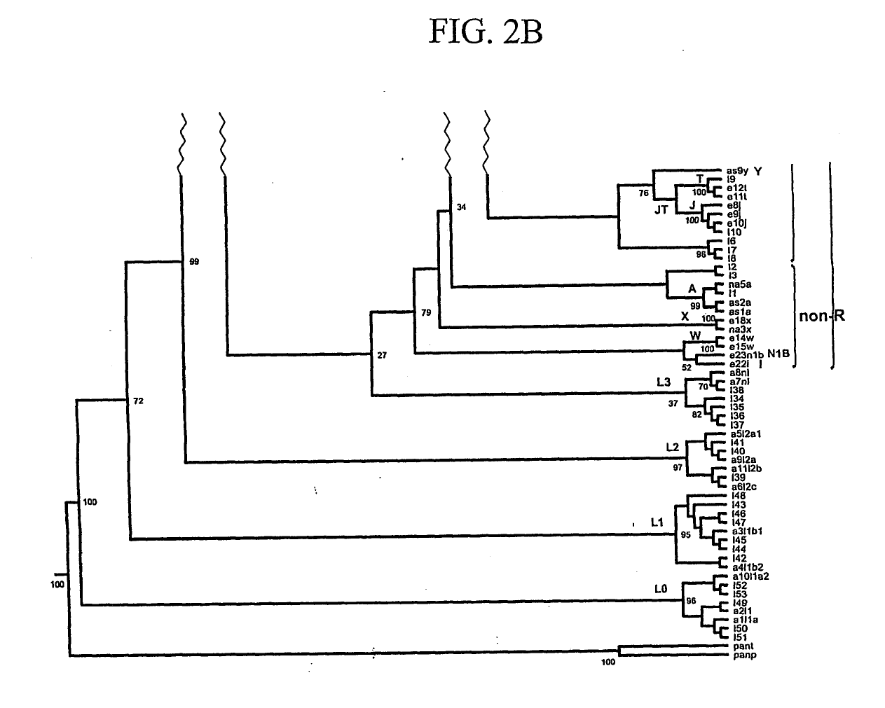

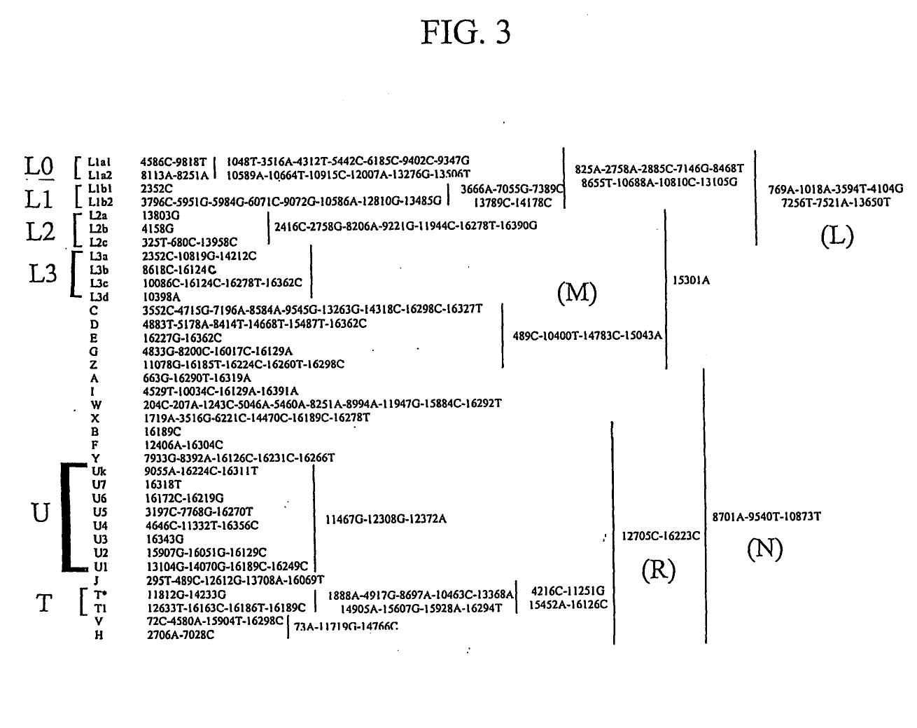

The nucleotide alleles listed in Table 3, including the Cambridge nucleotide alleles, are also useful for identifying mtDNA sequences associated with and diagnostic of human haplogroups. Example 2 summarizes phylogenetic analyses of the sequence data of the 103 individuals and the Cambridge sequence along with two chimpanzee mtDNA sequences. The results are shown in FIG. 1 in a cladogram. Calculations of the time since the most recent common ancestor (MRCA) are shown in Table 5. The 104 individuals were chosen from known haplogroups, and the corresponding haplogroups are labeled on the figure. Combining the sequence data of the 104 individuals with FIG. 1 and the geographic regions native to human haplogroups, as is known in the art, results in FIG. 2 (Example 3), which tracks human mtDNA migrations. Analysis of several mtDNA genomic sequences representing each haplogroup demonstrated which alleles are segregating within a haplogroup as well as which alleles are present in every individual within one or more haplogroups. The alleles that are present in every individual within each haplogroup are shown in FIG. 3 (Example 4). On the left, sub-haplogroups and haplogroups are listed. Macrohaplogroups are shown in parentheses. Nucleotide loci and alleles that are present in all the members of each group (sub-haplo or haplo) are listed. A vertical bar designates that all of the alleles to the right are present in all of the haplogroups and/or sub-haplogroups to the left. FIG. 3 is drawn as a cladogram. For example, FIG. 3 demonstrates that the macrohaplogroup (R) individuals all contain 12705C and 16223C, and no other individuals are known to have these alleles, therefore macro-haplogroup (R) can be diagnosed by identifying in a sample containing mtDNA, the presence of either 12705C or 16223C. Similarly, macro-haplogroup (N) can be diagnosed by identifying the presence of 8701A, 9540T, or 10873T.

Analysis of the data in FIG. 3 demonstrated sets of alleles useful for diagnosing the haplogroups (Example 5). These alleles are listed by haplogroup in Tables 6 and 7, and by sub-haplogroup in Tables 8 and 9. A set of alleles useful for diagnosing all of the haplogroups and sub-haplogroups in FIG. 3 is listed in Table 10. Table 10 lists the nucleotide loci in column one and the nucleotide alleles useful for diagnosing haplogroups in column two. Table 10 contains some alleles from the Cambridge sequence. There are many equivalent methods for diagnosing the haplogroups. Methods for diagnosing haplogroups that require testing only one or a few loci are listed in Example 5. The presence of only one particular allele is usually sufficient for diagnosing a haplogroup, however, often it is not known which locus needs to be tested. By determining the allele at each nucleotide locus listed in Table 10, the haplogroup of an unknown sample can be diagnosed. Alternatively, macro-haplogroups can be diagnosed or excluded first, thereby decreasing the number of loci that need to be tested to distinguish between the remaining, possible haplogroups. Alleles useful for diagnosing macro-haplogroups by methods that require testing only one or a few loci are included in Table 11. Further analysis of the data provided by this invention will demonstrate which sets of alleles identify additional sub-haplogroups and additional macro-haplogroups.

Diagnosing the haplogroup of a sample is useful in criminal investigations and forensic analyses. Identifying a sample as belonging to a particular haplogroup, and knowing which alleles have not been associated with a selected physiological condition and context, are useful when identifying novel alleles associated with a selected physiological condition, as described above and in Example 6. Diagnosing the haplogroup of a sample is also useful for identifying a novel allele associated with a selected physiological condition when the novel allele causes the physiological condition only in the genetic context of a particular haplogroup, as shown in Example 6. In example 6, the list of alleles associated with haplogroups found in Russia was used in the sequence analysis of two Russian LHON families. By eliminating alleles listed in Table 3, two novel mutations were identified that are associated with LHON. These new complex I mutations, 3635A and 4640C, are useful for diagnosing a predisposition to Leber Hereditary Optic Neuropathy (LHON).

Example 7 demonstrates the identification of a new primary LHON mutation, 10663C, in complex I, that appears to cause a predisposition to LHON only when associated with haplogroup J. Haplogroup J is defined by a nonsynonymous difference that is useful for diagnosing haplogroup J, 458T in ND5. This invention provides a method of diagnosing a person with a predisposition to LHON and/or to developing early onset blindness by identifying, in a sample containing mtDNA from the person, the nucleotide allele, or a synonymous nucleotide allele of 10663C and also identifying alleles diagnostic of haplogroup J, such as 458T in ND5. Because ND5458T is a missense mutation in all haplogroup J individuals, this particular mutation may be directly involved in causing LHON. ND1 304H is another missense mutation that is present in all haplogroup J individuals, and may also be directly involved in causing LHON. 458T is also present in haplogroup T individuals. Haplogroup J is also associated with a predisposition to centenaria and an extended lifespan. ND5 458T and ND1 304H may also be directly involved in causing the predisposition to centenaria and extended lifespan.

Example 8 demonstrates the importance of demographic factors in intercontinental mtDNA sequence radiation. Haplogroups are combined and separated into various populations for statistical analyses.

Previously in the art, it has been thought that polymorphisms in human mtDNA, such as the nucleotide alleles listed in Table 3, were neutral in all contexts and could not be associated with physiological conditions. It has been thought that differences in human mtDNA diversity associated with inter-continental migrations were due to random genetic drift (e.g. founder effects followed by rapid population expansion). In this invention, the biological and clinical significance of these human mtDNA polymorphisms are disclosed. The neutrality of the nucleotide alleles listed in Table 3 was tested using neutrality analysis (Examples 9-12).

Some of the nucleotide loci in Table 3 are located in the mitochondrial protein-coding genes (Table 2). Of those loci, some of the identified nucleotide alleles alter the protein encoded by the codon in which the nucleotide locus resides. This is determined using the mitochondrial codon usage table, as is known in the art. Nucleotide alleles that change an amino acid are called missense mutations, missense polymorphisms, or nonsynomymous differences. Missense polymorphisms alter the protein sequence relative to a compared sequence, but they still may be neutral because they do not affect the function of the encoded protein. Without performing biochemical studies on the affected proteins, statistical analyses can be performed to determine whether a polymorphism is neutral, whether evolution imposed selection on the encoding allele, and whether that selection is positive. This invention provides results of the statistical analyses of the polymorphisms in Table 3 and provides a list of which alleles are not neutral, and therefore evolutionarily significant.

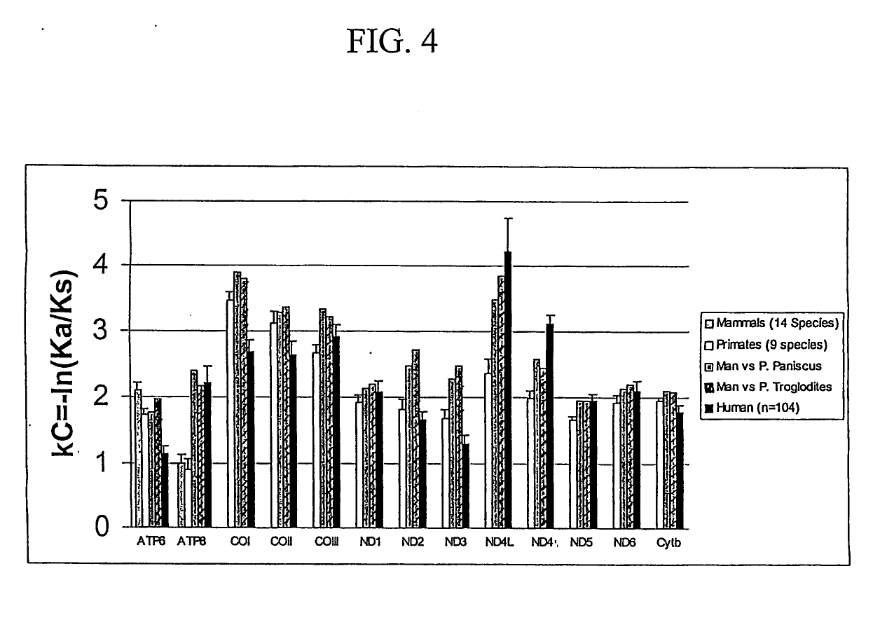

Neutrality testing of nucleotide alleles first requires neutrality testing of the genes containing those nucleotide alleles. Neutrality testing of one or more genes by comparing two sets of allelic genes from two intraspecific populations was performed, as described in Example 9. Haplogroups were combined to make populations for the comparison. In example 9, nucleotide alleles from the entire coding region of the mtDNA genome, representing haplogroups native to a geographic region, were combined to make a first population and first set of sequences. Nucleotide alleles of the entire coding region of the mtDNA genome, from haplogroups native to a different geographic region, were combined to make the second population and the second set of sequences. Nucleotide alleles were divided into those encoding synonymous and non-synonymous differences. The ratio of Kₐ/Kₛ for each gene, separated by the population containing the allele, is shown in Table 12. Neutrality testing of genes by comparing one set of at least two nucleotide alleles of at least one gene from one population of one species was performed in Example 10. In Example 10, sequences of the entire coding region of the mtDNA genome, of haplogroups in all geographic regions on earth, were combined to make one population and set of sequences for analysis. FIG. 4 shows the results of the comparison of one set of sequences from one population of only one species, 104 human sequences. Example 11 includes comparisons of sets of sequences between two populations, human vs. P. paniscus, human vs. P. troglodytes, human vs. eight other primate species, and human vs. thirteen mammalian species.

To identify an evolutionarily significant gene, two sets of nucleotide sequences, each set from a different population, are compared to each other. Nucleotide sequences representing parts of genes or one or more whole genes are useful. The sets of sequences are compared to each other by neutrality analysis. Differences in the sequences from each set are determined to be synonymous or nonsynonymous differences. The proportion of nonsynonymous differences is compared to the proportion of synonymous differences (Kₐ/Kₛ). The results of the analysis are compiled in a data set and the data set is analyzed, as is known in the art, to identify one or more evolutionarily significant genes. When the nonsynonymous differences occur significantly more often than is expected by chance than the synonymous differences, the gene or part of the gene is determined to be evolutionarily significant. When the synonymous differences occur significantly more often than is expected by chance than the nonsynonymous differences, the gene or part of the gene is determined to be conserved. When the ratio is as expected by chance, then there is no evidence of selection or evolutionary significance.

To identify an evolutionarily significant gene, only one set of nucleotide sequences (from only one population) may also be analyzed, e.g., the nucleotide sequences representative of humans living on one continent. When only one set of sequences is analyzed, the set must contain at least two corresponding nucleotide alleles (i.e., there must be sequence polymorphism). Corresponding sequences are sequences of the same gene or gene part from at least two individuals. The sequences from different individuals within the population must contain polymorphisms with respect to each other. Differences in the sequences relative to each other are determined to be synonymous or nonsynonymous. Neutrality analysis is performed to generate a data set. The data set is analyzed to identify an evolutionarily significant gene. If an analysis determines that none of the analyzed genes are evolutionarily significant, the set of nucleotide sequences can be increased, such as by increasing the size of the population from which the sequences are derived, to determine if one or more genes are evolutionarily significant in the enlarged population.

Example 12 is similar to example 9 except that the data is further analyzed by manipulating Kₐ/Kₛ to K_{C}. Examples 9-12 demonstrate that all but one mtDNA gene are not neutral and therefore are evolutionarily significant. Genes are determined to not be neutral by statistical significance tests known in the art. Some genes are only evolutionarily significant when comparing selected populations. For example, ND4 was demonstrated to be significant when comparing Native American sequences to African sequences and when comparing all human sequences to each other, but not when comparing European to African sequences. ND4L is the only mtDNA gene not shown to be evolutionarily significant by the current analyses. ND4L might be demonstrated to be evolutionarily significant by the methods of this invention using one or more different populations or using only part of the gene sequence. In examples 9-12, the entire sequence of each gene was used for analysis, however portions of genes are also useful in the methods of this invention. The statistical significance tests prevent too small a gene portion from being used to determine non-neutrality.

After identifying evolutionarily significant genes, evolutionarily significant nucleotide alleles can be identified. To identify an evolutionarily significant nucleotide allele, the steps for identifying an evolutionarily significant gene, using one or two populations, are performed with the addition of a step of analyzing the sequence data set to determine an evolutionarily significant nucleotide allele. An evolutionarily significant nucleotide allele is part of a sequence incoding an allelic amino acid in an evolutionarily significant gene or part of a gene. Examples 13 and 14 demonstrate identification of evolutionary significant nucleotide alleles and evolutionarily significant amino acid alleles in the evolutionarily significant genes identified in Examples 9-12. Evolutionarily significant amino acid alleles are the amino acids encoded by the codons containing evolutionarily significant nucleotide alleles. In these examples, nucleotides at loci not listed in Table 3 are identical to the Cambridge sequence so that the entire codon containing an evolutionarily significant nucleotide allele and the amino acid encoded by that codon can be determined. All nucleotide alleles that are part of a codon encoding the same amino acid as an evolutionarily significant amino acid allele identified herein, or identified by methods of this invention, are also evolutionarily significant and are intended to be within the scope of this invention. An evolutionarily significant amino acid allele may include more than one nucleotide allele, such as at two neighboring nucleotide loci. Evolutionarily significant nucleotide alleles and evolutionarily significant amino acid alleles in human mitochondrial sequences, identified by the methods of this invention, are listed in Table 14. In column one, Table 14 lists the gene containing the alleles, column two indicates the locus of the nucleotide allele, column three lists the Cambridge nucleotide allele at that nucleotide locus, column four lists a non-Cambridge allele of this invention, column five lists the amino acid encoded by the codon containing the Cambridge nucleotide allele (when other Cambridge nucleotides are present at the other nucleotide loci of the codon), and column six lists the amino acid encoded by the codon containing the non-Cambridge allele (when Cambridge nucleotides are present at the other nucleotide loci of the codon). Columns two, three, and four make the set of evolutionarily significant human mitochondrial nucleotide alleles. Columns two, five, and six make the set of evolutionarily significant human mitochondrial amino acid alleles. Table 14 designates the nucleotide locus of the listed alleles. For the amino acid alleles listed in columns five and six, the relevant loci are all three nucleotide loci in the encoding codon containing the nucleotide locus listed in column two.

To identify an evolutionarily significant amino acid allele, the steps for identifying an evolutionarily significant gene, using one or two populations, are performed with the addition of two steps: 1) analyzing the data set to determine an evolutionarily significant nucleotide allele; and 2) determining the encoded amino acid allele. An evolutionarily significant amino acid allele is a different amino acid, representing a nonsynonymous difference, relative to the corresponding amino acid allele against which it was compared, wherein the gene has been determined to be evolutionarily significant in the corresponding one or more populations.

In this invention it is demonstrated that amino acid substitution mutations (nonsynonymous differences) are much more common in human mtDNAs than would be expected by chance, and that most of them are evolutionarily significant. This invention demonstrates that these alleles have become fixed by selection. The mitochondrial genes encode proteins that are responsible for generating energy and for generating heat to maintain body temperature. As humans migrated to different parts of the world, they encountered changes in diet and climate. The high mutation rate of mtDNA and the central role of mitochondrial proteins in cellular energetics make the mtDNA an ideal system for permitting rapid mammalian adaptation to varying climatic and dietary conditions. The increased amino acid sequence variability that has been found among human mtDNA genes is due to the fact that natural selection favored mtDNA alleles that altered the coupling efficiency between the electron transport chain (ETC) and ATP synthesis, determined by the mitochondrial inner membrane proton gradient (AT). The coupling efficiency between the ETC and ATP synthesis is mediated to a considerable extent by the proton channel of the ATP synthase, which is composed of the mtDNA-encoded ATP6 protein and the nuclear DNA-encoded ATP9 protein. Mutations in the ATP6 gene, which create a more leaky ATP synthase proton channel, reduced ATP production but increased heat production for each calorie consumed. Such a change in energy balance was beneficial in a temperate or arctic climate, but deleterious in a tropical climate. Humans acquiring mtDNA alleles enabling better adaptation to the encountered changes in diet and climate experienced a higher genetic fitness and those alleles were selected for. In particular, these alleles were established genetically because they had an adaptive advantage as humans moved from the African tropics into the EurAsian temperate zone and on into the arctic (FIG. 2). The lack of recombination of the maternally inherited mtDNAs favored the rapid segregation, expression and adaptive selection of advantageous mtDNA alleles. The apparent non-randomness of the differences in non-synonymous versus synonymous mtDNA variation between continents demonstrates that selection also influenced inter-continental colonization. Random genetic hitchhiking, such as in the synonymous alleles, then resulted in identifiable continent-specific haplogroups.

Modern mtDNA variation has been shaped by adaptation as our ancestors moved into different environmental conditions. Variants that are advantageous in one climatic and dietary environment are maladaptive when individuals locate to a different environment. The methods of this invention associate mtDNA nucleotide alleles with haplogroups and combine this data with native haplogroup geographic regions as is known in the art, to diagnose individuals as having predispositions to late-onset clinical disorders such as obesity, diabetes, hypertension, and cardiovascular disease when those individuals live in climatic and dietary environments that are disadvantageous with respect to their mtDNA alleles. When humans having regional mtDNA alleles move into a different thermal and/or dietary environment from the one in which the alleles were selected, they are energetically imbalanced with their environment, and as a result are predisposed to having metabolic diseases such as diabetes, hypertension, cardiovascular disease, and other diseases known to the art to be associated with metabolism and mitochondrial functions. The above-mentioned late-onset clinical disorders are rapidly becoming epidemic around the world in members of our globally mobile society. This invention provides a method of diagnosing a human with a predisposition to a physiological condition such as, but not limited to, energetic imbalance, metabolic disease, abnormal energy metabolism, abnormal temperature regulation, abnormal oxidative phosphorylation, abnormal electron transport, obesity, amount of body fat, diabetes, hypertension, and cardiovascular disease. The method involves testing a sample containing mitochondrial nucleic acid from an individual in a geographic region to determine the haplogroup of the sample and therefore of the individual, comparing the haplogroup of the individual to the set of haplogroups known to be native to that geographic region, and diagnosing the individual human with a predisposition to the above-mentioned conditions if the haplogroup of the individual is not in the set of haplogroups native to that geographic region. This invention enables treatment of one of the above-mentioned conditions that is diagnosed by the above-mentioned method, comprising relocating the diagnosed human to a geographic region that is of similar climate as the region(s) native to the human's haplogroup and/or changing the diagnosed human's diet to more closely match the diet historically available in the region(s) native to the human's haplogroup.

The above-described method for diagnosing a predisposition to a physiological condition is also useful for associating an amino acid allele with the physiological condition The evolutionarily significant amino acid alleles present in the haplogroup of the diagnosed individual and not in the haplogroups native to the individual's geographic location are associated with the physiological condition by the methods of this invention. Amino acid alleles, and the corresponding nucleotide alleles, useful for diagnosing haplogroups, and the haplogroup they are useful for diagnosing, are listed in Table 15. The amino acid alleles and corresponding nucleotide alleles listed in Table 15, and synonymously coding nucleotide alleles, are associated with the above-mentioned physiological conditions. Table 15 lists the set of amino acid alleles useful for diagnosing haplogroups. Column one of Table 15 lists the gene, column two lists the nucleotide locus, column three lists the useful nucleotide allele, column four lists the useful amino acid allele encoded by the useful nucleotide allele when Cambridge nucleotides are present at the other nucleotide loci of the encoding codon, and column five lists the haplogroups or sub-haplogroups, in parentheses, that contain the corresponding alleles. The amino acid alleles (column four) can be identified by the codon containing the nucleotide locus (column two). For example, the proline in the ND1 gene is identified as ntl 3796 P, where ntl signifies the codon containing the nucleotide locus (ntl) 3796. When an individual of one of the haplogroups listed in column five of Table 15 is diagnosed with one of the above-mentioned physiological conditions by the above-mentioned method, the physiological condition is associated with the presence of one of the alleles listed in Table 15. When the haplogroup of the individual is haplogroup G, the amino acid allele likely to have caused the physiological condition is ntl 4833 A. When the haplogroup of the individual is haplogroup T, the amino acid allele is selected from the group consisting of ntl 14917 D, ntl 8701 T, and ntl 15452 I. When the haplogroup is haplogroup W, the amino acid allele is selected from the group consisting of ntl 5046 I, ntl 5460 T, ntl 8701 T, and ntl 15884 P. When the haplogroup is haplogroup D, the amino acid allele is selected from the group consisting of ntl 5178 M and ntl 8414 F. When the haplogroup is haplogroup L0, the amino acid allele is selected from the group consisting of ntl 5442 L, ntl 7146 A, ntl 9402 P, ntl 13105 V, and ntl 13276 V. When the haplogroup is haplogroup L1, the amino acid allele is selected from the group consisting of ntl 7146 A, ntl 7389H, ntl 13105 V, ntl 13789H, and ntl 14178 V. When the haplogroup is haplogroup C the amino acid allele is selected from the group consisting of ntl 8584 T and ntl 14318 S. When the haplogroup is selected from the group consisting of haplogroups A, I, X, B, F, Y, and U the amino acid allele is ntl 8701 T. When the haplogroup is haplogroup J the amino acid allele is selected from the group consisting of ntl 8701 T, ntl 13708 T, and ntl 15452 I. When the haplogroup is haplogroup selected from the group consisting of haplogroups V and H, the amino acid allele is selected from the group consisting of ntl 8701 T and ntl 14766 T.

Evolutionarily significant nucleotide and amino acid alleles also exist in nuclear-encoded ATP9 that are useful for diagnosing predisposition to an energy metabolism-related physiological condition such as energetic imbalance, metabolic disease, abnormal energy metabolism, abnormal temperature regulation, abnormal oxidative phosphorylation, abnormal electron transport, obesity, centenaria, diabetes, hypertension, and cardiovascular disease. These alleles may be identified by methods of this invention.

The evolutionarily significant amino acid alleles and corresponding nucleotide alleles are candidates for alleles causing a physiological condition for which a predisposition is diagnosable by the methods of this invention. The evolutionarily significant amino acid and nucleotide alleles identified by the methods of this invention (Table 19) are useful for gene therapy and mitochondrial replacement therapy to treat the corresponding physiological conditions. The evolutionarily significant genes, amino acid alleles, and nucleotide alleles identified by the methods of this invention are useful for identifying targets for traditional therapy, and for designing corresponding therapeutic agents. The evolutionarily significant genes and amino acid and nucleotide changes identified by the methods of this invention are useful for generating animal models of the corresponding human physiological conditions.

As is known to the art, individuals may contain more than one mitochondrial DNA allele at any given nucleotide locus. One cell contains many mitochondria, and one cell or different cells within one organism may contain genetically different mitochondria. Heteroplasmy is the occurrence of more than one type of mitochondria in an individual or sample. Varying degrees of heteroplasmy are associated with varying degrees of the physiological conditions described herein. Heteroplasmy may be identified by means known to the art, and the severity of the physiological condition associated with specific nucleotide alleles is expected to vary with the percentage of such associated alleles within the individual.

The methods of this invention are used to analyze the human mitochondrial genome in the listed examples, but the methods are also useful for analyzing other genomes and other species. The methods of this invention are useful for identifying evolutionarily significant protein-coding genes and the correspondingly encoded mutations in other genomes in addition to mitochondrial genomes, such as in nuclear and chloroplast genomes. Using human haplogroups as populations (FIG. 1), the methods of this invention are useful for identifying evolutionarily significant protein-coding genes and the corresponding evolutionarily significant alleles in human nuclear genes. The methods of this invention are also useful for identifying evolutionarily significant protein-coding genes and the corresponding alleles in many species. For example, the methods of this invention are applicable to varieties of beef or dairy cattle, or pig lines. Corn lines are divisible by phenotypic and/or molecular markers into heterotic groups that are useful populations in the methods of this invention. Using corn heterotic groups as populations, the methods of this invention are useful for identifying evolutionarily significant protein-coding genes and the corresponding mutations in the nuclear, chloroplast, and mitochondrial genomes of corn.

This invention provides isolated nucleic acid molecules containing novel nucleotide alleles of this invention in libraries. The libraries contain at least two such molecules. Preferably the molecules have unique sequences. The molecules typically have a length from about 7 to about 30 nucleotides. “About” as used herein means within about 10% (e.g., “about 30 nucleotides” means 27-33 nucleotides). However, the molecules may be longer, such as about 50 nucleotides long. A library of this invention contains at least two isolated nucleic acid molecules each containing at least one non-Cambridge nucleotide allele of this invention. A library of this invention may contain at least ten, twenty-five, fifty, 100, 500 or more isolated nucleic acid molecules, at least one of which contains a nucleotide allele of this invention. A library of this invention may contain molecules having at least two to all of the nucleotide alleles of this invention, including synonymous codings of evolutionarily significant amino acid alleles. The nucleotide alleles of this invention are defined by a nucleotide locus, the nucleotide location in the human mitochondrial genome, and by the A G C T (or U) nucleotide. An isolated nucleic acid molecule, in a library of this invention, can be identified as containing a nucleotide allele of this invention, because the nucleotide allele of this invention is bounded on at least one side by its context in the mitochondrial genome. Statistically, to be unique in the human mitochondrial genome, such a molecule would need to be at least about seven nucleotides long. Statistically, to be unique in the total human genome, including the mitochondrial genome, such a molecule would need to be at least about fifteen nucleotides long. Examples of isolated nucleic acid molecules of this invention are molecules containing the following nucleotide alleles: 1) Cambridge alleles at human mtDNA nucleotide loci 168-170, non-Cambridge alleles at locus 171A, and Cambridge alleles at human mtDNA nucleotide loci 172-174; and 2) Cambridge alleles at 11940-11946, non-Cambridge alleles at 11947G, and Cambridge alleles at 11948-11954. An isolated nucleic acid molecule of this invention may contain more than one nucleotide allele of this invention. The nucleotide allele of this invention may be at any position in the isolated nucleic acid molecule. Often it is useful to have the relevant nucleotide allele in the center of the isolated nucleic acid molecule or on the 3′ end of the molecule. Isolated nucleic acid molecules of this invention are useful for interrogating, determining the presence or absence of, a nucleotide allele at the corresponding nucleotide locus in the mitochondrial genome in a sample containing mitochondrial nucleic acid from a human, using any method known in the art. Methods for determining the presence of absence of the nucleotide allele include allele-specific PCR and nucleic acid array hybridization or sequencing.

The alleles and libraries of this invention are useful for designing probes for nucleic acid arrays. This invention provides nucleic acid arrays having two or more nucleic acid molecules or spots (each spot comprising a plurality of substantially identical isolated nucleic acid molecules), each molecule having the sequence of an allele of this invention. The molecules on the arrays of this invention are usually about 7 to about 30 nucleotides long. The arrays are useful for detecting the presence or absence of alleles. Arrays of this invention are also useful for sequencing human mtDNA. Alleles may be selected from sets of nucleotide alleles including human mtDNA nucleotide alleles, non-Cambridge human mtDNA nucleotide alleles, human mtDNA nucleotide alleles in 48 genomes and the Cambridge sequence, non-Cambridge human mtDNA nucleotide alleles in 48 genomes, nucleotide alleles useful for diagnosing human haplogroups and macro-haplogroups, nucleotide alleles useful for diagnosing human haplogroups, and evolutionarily significant human mitochondrial nucleotide alleles as listed in the various Tables and portions of tables hereof. Arrays of this invention may contain molecules capable of interrogating all of the alleles in one of the above-mentioned sets of alleles. A genotyping array useful for detecting sequence polymorphisms, such as are provided by this invention, are similar to Affymetrix (Santa Clara, Calif., USA) genotyping arrays containing a Perfect Match probe (PM) and a corresponding Mismatch probe (MM). A PM probe could comprise a non-Cambridge allele at a selected nucleotide locus and the corresponding MM probe could comprise the corresponding Cambridge allele at the selected nucleotide locus. Arrays of this invention include sequencing arrays for human mtDNA.

As used herein, “array” refers to an ordered set of isolated nucleic acid molecules or spots consisting of pluralities of substantially identical isolated nucleic acid molecules. Preferably the molecules are attached to a substrate. The spots or molecules are ordered so that the location of each (on the substrate) is known and the identity of each is known. Arrays on a microscale can be called microarrays. Microarays on solid substrates, such as glass or other ceramic slides, can be called gene chips or chips.

Arrays are preferably printed on solid substrates. Before printing, substrates such as glass slides are prepared to provide a surface useful for binding, as is known to the art. Arrays may be printed using any printing techniques and machines known in the art. Printing involves placing the probes on the substrate, attaching the probes to the substrate, and blocking the substrate to prevent non-specific hybridization Spots are printed at known locations. Arrays may be printed on glass microscope slides. Alternatively, probes may be synthesized in known positions on prepared solid substrates (Affymetrix, Santa Clara, Calif., USA).

Arrays of this invention may contain as few as two spots, or more than about ten spots, more than about twenty-five spots, more than about one hundred spots, more than about 1000 spots, more than about 65,000 spots, or up to about several hundred thousand spots.

Using microarrays may require amplification of target sequences (generation of multiple copies of the same sequence) of sequences of interest, such as by PCR or reverse transcription. As the nucleic acid is copied, it is tagged with a fluorescent label that emits light like a light bulb. The labeled nucleic acid is introduced to the microarray and allowed to react for a period of time. This nucleic acid sticks to, or hybridizes, with the probes on the array when the probe is sufficiently complementary to the labeled, amplified, sample nucleic acid. The extra nucleic acid is washed off of the array, leaving behind only the nucleic acid that has bound to the probes. By obtaining an image of the array with a fluorescent scanner and using software to analyze the hybridized array image, it can be determined if, and to what extent, genes are switched on and off, or whether or not sequences are present, by comparing fluorescent intensities at specific locations on the array. The intensity of the signal indicates to what extent a sequence is present. In expression arrays, high fluorescent signals indicate that many copies of a gene are present in a sample, and lower fluorescent signal shows a gene is less active. By selecting appropriate hybridization conditions and probes, this technique is useful for detecting single nucleotide polymorphisms (SNPs) and for sequencing. Methods of designing and using microarrays are continuously being improved (Relogio, A. et al. (2002) Nuc. Acids. Res. 30(11): e51; Iwasaki, H et al. (2002) DNA Res. 9(2):59-62; and Lindroos, K. et al. (2002) Nuc. Acids. Res. 30(14):E70).

Arrays of this invention may be made by any array synthesis methods known in the art such as spotting technology or solid phase synthesis. Preferably the arrays of this invention are synthesized by solid phase synthesis using a combination of photolithography and combinatorial chemistry. Some of the key elements of probe selection and array design are common to the production of all arrays. Strategies to optimize probe hybridization, for example, are invariably included in the process of probe selection. Hybridization under particular pH, salt, and temperature conditions can be optimized by taking into account melting temperatures and by using empirical rules that correlate with desired hybridization behaviors. Computer models may be used for predicting the intensity and concentration-dependence of probe hybridization.

Detecting a particular polymorphism can be accomplished using two probes. One probe is designed to be perfectly complementary to a target sequence, and a partner probe is generated that is identical except for a single base mismatch in its center. In the Affymetrix system, these probe pairs are called the Perfect Match probe (PM) and the Mismatch probe (MM). They allow for the quantitation and subtraction of signals caused by non-specific cross-hybridization. The difference in hybridization signals between the partners, as well as their intensity ratios, serve as indicators of specific target abundance, and consequently of the sequence.

Arrays can rely on multiple probes to interrogate individual nucleotides in a sequence. The identity of a target base can be deduced using four identical probes that vary only in the target position, each containing one of the four possible bases. Alternatively, the presence of a consensus sequence can be tested using one or two probes representing specific alleles. To genotype heterozygous or genetically mixed samples, arrays with many probes can be created to provide redundant information, resulting in unequivocal genotyping.

Probes fixed on solid substrates and targets (nucleotide sequences in the sample) are combined in a hybridization buffer solution and held at an appropriate temperature until annealing occurs. Thereafter, the substrate is washed free of extraneous materials, leaving the nucleic acids on the target bound to the fixed probe molecules allowing for detection and quantitation by methods known in the art such as by autoradiograph, liquid scintillation counting, and/or fluorescence. As improvements are made in hybridization and detection techniques, they can be readily applied by one of ordinary skill in the art. As is well known in the art, if the probe molecules and target molecules hybridize by forming a strong non-covalent bond between the two molecules, it can be reasonably assumed that the probe and target nucleic acid are essentially identical, or almost completely complementary if the annealing and washing steps are carried out under conditions of high stringency. The detectable label provides a means for determining whether hybridization has occurred.

When using oligonucleotides or polynucleotides as hybridization probes, the probes may be labeled. In arrays of this invention, the target may instead be labeled by means known to the art. Target may be labeled with radioactive or non-radioactive labels. Targets preferably contain fluorescent labels.

Various degrees of stringency of hybridization can be employed. The more stringent the conditions are, the greater the complementarity that is required for duplex formation. Stringency can be controlled by temperature, probe concentration, probe length, ionic strength, time, and the like. Hybridization experiments are often conducted under moderate to high stringency conditions by techniques well know in the art, as described, for example in Keller, G. H., and M. M. Manak (1987) DNA Probes, Stockton Press, New York, N.Y., pp. 169-170, hereby incorporated by reference. However, sequencing arrays typically use lower hybridization stringencies, as is known in the art.

Moderate to high stringency conditions for hybridization are known to the art. An example of high stringency conditions for a blot are hybridizing at 68° C. in 5×SSC/5× Denhardt's solution/0.1% SDS, and washing in 0.2×SSC/0.1% SDS at room temperature. An example of conditions of moderate stringency are hybridizing at 68° C. in 5×SSC/5× Denhardt's solution/0.1% SDS and washing at 42° C. in 3×SSC. The parameters of temperature and salt concentration can be varied to achieve the desired level of sequence identity between probe and target nucleic acid. See, e.g., Sambrook et al. (1989) vide infra or Ausubel et al. (1995) Current Protocols in Molecular Biology, John Wiley & Sons, NY, N.Y., for further guidance on hybridization conditions.

The melting temperature is described by the following formula (Beltz, G. A. et al., [1983] Methods of Enzymology, R. Wu, L. Grossman and K. Moldave [Eds.] Academic Press, New York 100:266-285).

Tₘ=81.5o C+16.6 Log[Na+]+0.41(+G+C)−0.61(% formamide)−600/length of duplex in base pairs.

Washes can typically be carried out as follows: twice at room temperature for 15 minutes in 1×SSPE, 0.1% SDS (low stringency wash), and once at TM-20° C. for 15 minutes in 0.2×SSPE, 0.1% SDS (moderate stringency wash).

Nucleic acid useful in this invention can be created by Polymerase Chain Reaction (PCR) amplification. PCR products can be confirmed by agarose gel electrophoresis. PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. [1985] Science 230:1350-1354). PCR is used to enzymatically amplify a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Since the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes that can be used are known to those skilled in the art.

Polynucleotide sequences of the present invention can be truncated and/or mutated such that certain of the resulting fragments and/or mutants of the original full-length sequence can retain the desired characteristics of the full-length sequence. A wide variety of restriction enzymes that are suitable for generating fragments from larger nucleic acid molecules are well known. In addition, it is well known that Bal31 exonuclease can be conveniently used for time-controlled limited digestion of DNA. See, for example, Maniatis (1982) Molecular Cloning: A Laboratory Manual, Cold Spring Harbor Laboratory, New York, pages 135-139, incorporated herein by reference. See also Wei et al. (1983) J. Biol. Chem. 258:13006-13512. By use of Bal31 exonuclease (commonly referred to as “erase-a-base” procedures), the ordinarily skilled artisan can remove nucleotides from either or both ends of the subject nucleic acids to generate a wide spectrum of fragments that are functionally equivalent to the subject nucleotide sequences. One of ordinary skill in the art can, in this manner, generate hundreds of fragments of controlled, varying lengths from locations all along the original molecule. The ordinarily skilled artisan can routinely test or screen the generated fragments for their characteristics and determine the utility of the fragments as taught herein. It is also well known that the mutant sequences can be easily produced with site-directed mutagenesis. See, for example, Larionov, O. A. and Nikiforov, V. G. (1982) Genetika 18(3):349-59; and Shortle, D. et al., (1981) Annu. Rev. Gene. 15:265-94, both incorporated herein by reference. The skilled artisan can routinely produce deletion-, insertion-, or substitution-type mutations and identify those resulting mutants that contain the desired characteristics of wild-type sequences, or fragments thereof.

Percent sequence identity of two nucleic acids may be determined using the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (NBLAST and XBLAST) are used. See http://www.ncbi.nih.gov.

Standard techniques for cloning, DNA isolation, amplification and purification, for enzymatic reactions involving DNA ligase, DNA polymerase, restriction endonucleases and the like, and various separation techniques useful herein are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (Ed.) (1985) DNA Cloning Vol. I and II, IRL Press, Oxford, UK; Hames and Higgins (Eds.) (1985) Nucleic Acid Hybridization, IRL Press, Oxford, UK; Setlow and Hollaender (1979) Genetic Engineering: Principles and Methods, Vols. 1-4, Plenum Press, New York; and Ausubel et al. (1992) Current Protocols in Molecular Biology, Greene/Wiley, New York, N.Y. Abbreviations and nomenclature, where employed, are deemed standard in the field and commonly used in professional journals such as those cited herein.

This invention provides machine-readable storage devices and program storage devices having data and methods for diagnosing haplogroups and physiological conditions. One program storage device provided by this invention contains the program steps: a) determining the haplogroup of a sample from an individual using nucleotide sequence data from nucleic acid in the sample; b) associating the haplogroup with information identifying the geographic region of the individual; c) comparing the haplogroup and geographic region of the sample to the set of haplogroups native to the geographic region of the individual; and d) diagnosing the individual with a predisposition to an energy metabolism-related physiological condition if the haplogroup of the individual is not within the set of haplogroups native to the geographic region of the individual; all said program steps being encoded in machine readable form, and all said information encoded in machine readable form. This invention also provides a data set, encoded in machine-readable form, containing nucleotide alleles listed in Table 19, with each allele associated with encoded information identifying a physiological condition in humans. These physiological conditions are energy-metabolism-related conditions including energetic imbalance, metabolic disease, abnormal energy metabolism, abnormal temperature regulation, abnormal oxidative phosphorylation, abnormal electron transport, obesity, amount of body fat, diabetes, hypertension, and cardiovascular disease. This storage device may also contain information associating each allele with one or more native geographic regions. A program storage device provided by this invention contains input means for inputting the haplogroup of an individual and the geographic region of that individual, and contains information associating alleles with native geographic regions, and program steps for diagnosing the individual with a predisposition to a physiological condition. A storage device containing a data set in machine readable form provided by this invention may include encoded information comprising amino acid alleles listed in Table 19, with each allele associated with a physiological condition in humans.

It will be appreciated by those of ordinary skill in the art that populations, subpopulations, organelles, and amino acid and nucleotide sequence comparison methods, neutrality test methods, nucleotide sequencing methods, codons, samples, sample collection techniques, sample preparation techniques, probes, probe generation techniques, genes involved in mitochondrial biology, hybridization techniques, array printing techniques, physiological conditions, cell lines, mutant strains, organisms, tissues, solid substrates, machine-readable storage devices, program devices, and methods of data analyses other than those specifically disclosed herein are available in the art and can be employed in the practice of this invention. All art-known functional equivalents are intended to be encompassed within the scope of this invention.

The following examples are provided for illustrative purposes, and are not intended to limit the scope of the invention as claimed here. Any variations in the compositions and methods exemplified that occur to the skilled artisan are intended to fall within the scope of the present invention.

### EXAMPLES

### Example 1

This invention provides human mtDNA polymorphisms found in all the major human haplogroups. Table 3 shows naturally occurring nucleotide alleles identified in the complete mtDNA sequences of 103 individuals, as compared to the mtDNA Cambridge sequence. All nucleotide sequences not listed are identical to the Cambridge sequence. Nucleotide alleles previously known to be associated with disease conditions, such as those listed in Table 1, are not listed in Table 3. Some deletion or rearrangement polymorphisms have also been excluded. All polymorphisms listed are nucleotide substitutions except for a nine-adenine nucleotide deletion at positions 8271-8279.

TABLE 3
Human MtDNA Nucleotide Alleles

| | | non- |
|---|---|---|
| nucleotide | Cambridge | Cambridge |
| locus | alleles | alleles |
| 64 | C | T |
| 72 | T | C |
| 73 | A | G |
| 89 | T | C |
| 93 | A | G |
| 95 | A | C |
| 114 | C | T |
| 143 | G | A |
| 146 | T | C |
| 150 | C | T |
| 151 | C | T |
| 152 | T | C |
| 153 | A | G |
| 171 | G | A |
| 180 | T | C |
| 182 | C | T |
| 183 | A | G |
| 185 | G | A |
| 185 | G | T |
| 186 | C | A |
| 189 | A | C |
| 189 | A | G |
| 194 | C | T |
| 195 | T | A |
| 195 | T | C |
| 198 | C | T |
| 199 | T | C |
| 200 | A | G |
| 204 | T | C |
| 207 | G | A |
| 208 | T | C |
| 210 | A | G |
| 212 | T | C |
| 215 | A | G |
| 217 | T | C |
| 225 | G | A |
| 227 | A | G |
| 228 | G | A |
| 235 | A | G |
| 236 | T | C |
| 247 | G | A |
| 250 | T | C |
| 252 | T | C |
| 263 | A | G |
| 291 | A | G |
| 295 | C | T |
| 297 | A | G |
| 316 | G | A |
| 317 | C | A |
| 317 | C | G |
| 320 | C | T |
| 325 | C | T |
| 340 | C | T |
| 357 | A | G |
| 373 | A | G |
| 400 | T | G |
| 408 | T | A |
| 418 | C | T |
| 456 | C | T |
| 462 | C | T |
| 465 | C | T |
| 467 | C | T |
| 471 | T | C |
| 480 | T | C |
| 482 | T | C |
| 489 | T | C |
| 493 | A | G |
| 499 | G | A |
| 508 | A | G |
| 593 | T | C |
| 597 | C | T |
| 663 | A | G |
| 678 | T | C |
| 680 | T | C |
| 709 | G | A |
| 710 | T | C |
| 721 | T | C |
| 750 | A | G |
| 769 | G | A |
| 825 | T | A |
| 827 | A | G |
| 850 | T | C |
| 921 | T | C |
| 930 | G | A |
| 961 | T | C |
| 961 | T | G |
| 1018 | G | A |
| 1041 | A | G |
| 1048 | C | T |
| 1119 | T | C |
| 1189 | T | C |
| 1243 | T | C |
| 1290 | C | T |
| 1382 | A | C |
| 1406 | T | C |
| 1415 | G | A |
| 1420 | T | C |
| 1438 | A | G |
| 1442 | G | A |
| 1503 | G | A |
| 1598 | G | A |
| 1700 | T | C |
| 1703 | C | T |
| 1706 | C | T |
| 1709 | G | A |
| 1715 | C | T |
| 1719 | G | A |
| 1736 | A | G |
| 1738 | T | C |
| 1780 | T | C |
| 1811 | A | G |
| 1888 | G | A |
| 1927 | G | A |
| 2000 | C | T |
| 2060 | A | G |
| 2092 | C | T |
| 2245 | A | C |
| 2245 | A | G |
| 2263 | C | A |
| 2308 | A | G |
| 2332 | C | T |
| 2352 | T | C |
| 2358 | A | G |
| 2380 | C | T |
| 2416 | T | C |
| 2483 | T | C |
| 2581 | A | G |
| 2639 | C | T |
| 2650 | C | T |
| 2706 | A | G |
| 2755 | A | G |
| 2758 | G | A |
| 2768 | A | G |
| 2789 | C | T |
| 2792 | A | G |
| 2834 | C | T |
| 2836 | C | A |
| 2857 | T | C |
| 2863 | T | C |
| 2885 | T | C |
| 3010 | G | A |
| 3083 | T | C |
| 3197 | T | C |
| 3200 | T | A |
| 3202 | T | C |
| 3204 | C | T |
| 3206 | C | T |
| 3221 | A | G |
| 3290 | T | C |
| 3308 | T | C |
| 3316 | G | A |
| 3372 | T | C |
| 3394 | T | C |
| 3438 | G | A |
| 3450 | C | T |
| 3480 | A | G |
| 3505 | A | G |
| 3513 | C | T |
| 3516 | C | A |
| 3516 | C | G |
| 3547 | A | G |
| 3549 | C | T |
| 3552 | T | A |
| 3552 | T | C |
| 3565 | A | G |
| 3594 | C | T |
| 3644 | T | C |
| 3666 | G | A |
| 3693 | G | A |
| 3699 | C | T |
| 3720 | A | G |
| 3756 | A | G |
| 3796 | A | G |
| 3796 | A | T |
| 3796 | A | C |
| 3808 | A | G |
| 3816 | A | G |
| 3834 | G | A |
| 3843 | A | G |
| 3847 | T | C |
| 3866 | T | C |
| 3918 | G | A |
| 3921 | C | A |
| 3927 | A | G |
| 3970 | C | T |
| 3981 | A | G |
| 4025 | C | T |
| 4040 | C | T |
| 4044 | A | G |
| 4048 | G | A |
| 4086 | C | T |
| 4104 | A | G |
| 4117 | T | C |
| 4122 | A | G |
| 4123 | A | G |
| 4158 | A | G |
| 4203 | A | G |
| 4216 | T | C |
| 4221 | C | T |
| 4225 | A | G |
| 4232 | T | C |
| 4248 | T | C |
| 4312 | C | T |
| 4336 | T | C |
| 4370 | T | C |
| 4388 | A | G |
| 4454 | T | A |
| 4491 | G | A |
| 4506 | A | G |
| 4508 | C | T |
| 4512 | G | A |
| 4529 | A | C |
| 4529 | A | T |
| 4541 | G | A |
| 4580 | G | A |
| 4586 | T | C |
| 4596 | G | A |
| 4646 | T | C |
| 4655 | G | A |
| 4688 | T | C |
| 4695 | T | C |
| 4715 | A | G |
| 4742 | T | C |
| 4767 | A | G |
| 4769 | A | G |
| 4820 | G | A |
| 4824 | A | G |
| 4833 | A | G |
| 4841 | G | A |
| 4883 | C | T |
| 4907 | T | C |
| 4917 | A | G |
| 4960 | C | T |
| 4977 | T | C |
| 4994 | A | G |
| 5004 | T | C |
| 5027 | C | T |
| 5036 | A | G |
| 5043 | G | T |
| 5046 | G | A |
| 5063 | T | C |
| 5096 | T | C |
| 5108 | T | C |
| 5147 | G | A |
| 5153 | A | G |
| 5178 | C | A |
| 5231 | G | A |
| 5237 | G | A |
| 5255 | C | T |
| 5262 | G | A |
| 5263 | C | T |
| 5285 | A | G |
| 5300 | C | T |
| 5330 | C | A |
| 5331 | C | A |
| 5390 | A | G |
| 5393 | T | C |
| 5417 | G | A |
| 5426 | T | C |
| 5442 | T | C |
| 5460 | G | A |
| 5465 | T | C |
| 5471 | G | A |
| 5492 | T | C |
| 5495 | T | C |
| 5580 | T | C |
| 5581 | A | G |
| 5601 | C | T |
| 5603 | C | T |
| 5606 | C | T |
| 5633 | C | T |
| 5655 | T | C |
| 5711 | A | G |
| 5773 | G | A |
| 5811 | A | G |
| 5814 | T | C |
| 5821 | G | A |
| 5826 | T | C |
| 5843 | A | G |
| 5951 | A | G |
| 5984 | A | G |
| 5987 | C | T |
| 6026 | G | A |
| 6029 | C | T |
| 6045 | C | T |
| 6071 | T | C |
| 6077 | C | T |
| 6104 | C | T |
| 6150 | G | A |
| 6152 | T | C |
| 6164 | C | T |
| 6167 | T | C |
| 6182 | G | A |
| 6185 | T | C |
| 6185 | T | C |
| 6221 | T | C |
| 6227 | T | C |
| 6253 | T | C |
| 6257 | G | A |
| 6324 | G | A |
| 6366 | G | A |
| 6371 | C | T |
| 6392 | T | C |
| 6473 | C | T |
| 6491 | C | A |
| 6524 | T | C |
| 6548 | C | T |
| 6587 | C | T |
| 6607 | T | C |
| 6680 | T | C |
| 6713 | C | T |
| 6719 | T | C |
| 6734 | G | A |
| 6752 | A | G |
| 6770 | A | G |
| 6776 | T | C |
| 6815 | T | C |
| 6827 | T | C |
| 6875 | C | A |
| 6938 | C | T |
| 6962 | G | A |
| 6989 | A | G |
| 7028 | C | T |
| 7052 | A | G |
| 7055 | A | G |
| 7058 | T | A |
| 7076 | A | G |
| 7146 | A | G |
| 7154 | A | G |
| 7175 | T | C |
| 7196 | C | A |
| 7202 | A | G |
| 7226 | G | A |
| 7256 | C | T |
| 7257 | A | G |
| 7271 | A | G |
| 7274 | C | T |
| 7319 | T | C |
| 7337 | G | A |
| 7347 | G | A |
| 7389 | T | C |
| 7403 | A | G |
| 7424 | A | G |
| 7444 | G | A |
| 7476 | C | T |
| 7493 | C | T |
| 7521 | G | A |
| 7561 | T | C |
| 7571 | A | G |
| 7600 | G | A |
| 7624 | T | A |
| 7645 | T | C |
| 7648 | C | T |
| 7660 | T | C |
| 7664 | G | A |
| 7673 | A | G |
| 7675 | C | T |
| 7693 | C | T |
| 7694 | C | T |
| 7697 | G | A |
| 7744 | T | C |
| 7765 | A | G |
| 7768 | A | G |
| 7771 | A | G |
| 7858 | C | T |
| 7861 | T | C |
| 7864 | C | T |
| 7867 | C | T |
| 7933 | A | G |
| 7948 | C | T |
| 7999 | T | C |
| 8014 | A | G |
| 8020 | G | A |
| 8027 | G | A |
| 8080 | C | T |
| 8087 | T | C |
| 8113 | C | A |
| 8142 | C | T |
| 8149 | A | G |
| 8152 | G | A |
| 8155 | G | A |
| 8185 | T | C |
| 8200 | T | C |
| 8206 | G | A |
| 8248 | A | G |
| 8251 | G | A |
| 8260 | T | C |
| 8269 | G | A |
| 8271-8279 | A | DEL |
| 8286 | T | C |
| 8292 | G | A |
| 8298 | T | C |
| 8344 | A | G |
| 8387 | G | A |
| 8389 | A | G |
| 8392 | G | A |
| 8404 | T | C |
| 8414 | C | T |
| 8428 | C | T |
| 8448 | T | C |
| 8460 | A | G |
| 8468 | C | T |
| 8472 | C | T |
| 8473 | T | C |
| 8485 | G | A |
| 8545 | G | A |
| 8553 | C | T |
| 8563 | A | G |
| 8566 | A | G |
| 8577 | A | G |
| 8584 | G | A |
| 8618 | T | C |
| 8655 | C | T |
| 8697 | G | A |
| 8701 | A | G |
| 8703 | C | T |
| 8705 | T | C |
| 8709 | C | T |
| 8721 | A | G |
| 8733 | T | C |
| 8764 | G | A |
| 8781 | C | A |
| 8784 | A | G |
| 8790 | G | A |
| 8793 | T | C |
| 8794 | C | T |
| 8805 | A | G |
| 8836 | A | G |
| 8838 | G | A |
| 8856 | G | A |
| 8860 | A | G |
| 8875 | T | C |
| 8877 | T | C |
| 8911 | T | C |
| 8913 | A | G |
| 8928 | T | C |
| 8943 | C | T |
| 8962 | A | G |
| 8994 | G | A |
| 9042 | C | T |
| 9053 | G | A |
| 9055 | G | A |
| 9072 | A | G |
| 9077 | T | C |
| 9090 | T | C |
| 9093 | A | C |
| 9103 | T | C |
| 9114 | A | G |
| 9120 | A | G |
| 9123 | G | A |
| 9123 | G | A |
| 9136 | A | G |
| 9151 | A | G |
| 9156 | A | G |
| 9174 | T | C |
| 9221 | A | G |
| 9237 | G | A |
| 9242 | A | G |
| 9248 | C | T |
| 9263 | A | G |
| 9272 | C | T |
| 9296 | C | T |
| 9311 | T | C |
| 9325 | T | C |
| 9335 | C | T |
| 9347 | A | G |
| 9355 | A | G |
| 9356 | C | T |
| 9377 | A | G |
| 9402 | A | C |
| 9449 | C | T |
| 9456 | A | G |
| 9477 | G | A |
| 9509 | T | C |
| 9536 | C | T |
| 9540 | T | C |
| 9545 | A | G |
| 9548 | G | A |
| 9554 | G | A |
| 9559 | C | G |
| 9575 | G | A |
| 9591 | G | A |
| 9599 | C | T |
| 9632 | A | G |
| 9647 | T | C |
| 9667 | A | G |
| 9682 | T | C |
| 9698 | T | C |
| 9755 | G | A |
| 9818 | C | T |
| 9822 | C | A |
| 9824 | T | A |
| 9911 | C | T |
| 9932 | G | A |
| 9950 | T | C |
| 9957 | T | C |
| 9966 | G | A |
| 9977 | T | C |
| 10034 | T | C |
| 10086 | A | G |
| 10086 | A | C |
| 10115 | T | C |
| 10118 | T | C |
| 10142 | C | T |
| 10151 | A | G |
| 10152 | G | C |
| 10172 | G | A |
| 10182 | G | C |
| 10197 | G | A |
| 10238 | T | C |
| 10253 | T | C |
| 10256 | T | C |
| 10310 | G | A |
| 10313 | A | G |
| 10321 | T | C |
| 10325 | G | A |
| 10358 | A | G |
| 10370 | T | C |
| 10398 | A | G |
| 10400 | C | T |
| 10410 | T | C |
| 10414 | G | T |
| 10427 | G | A |
| 10463 | T | C |
| 10499 | A | G |
| 10505 | T | C |
| 10550 | A | G |
| 10586 | G | A |
| 10589 | G | A |
| 10609 | T | C |
| 10637 | C | T |
| 10640 | T | C |
| 10646 | G | A |
| 10659 | C | T |
| 10664 | C | T |
| 10667 | T | C |
| 10688 | G | A |
| 10736 | C | T |
| 10790 | T | C |
| 10792 | A | G |
| 10793 | C | T |
| 10804 | A | G |
| 10810 | T | C |
| 10819 | A | G |
| 10828 | T | C |
| 10873 | T | C |
| 10876 | A | G |
| 10894 | C | T |
| 10915 | T | C |
| 10920 | C | T |
| 10939 | C | T |
| 10966 | T | C |
| 10984 | C | G |
| 11002 | A | G |
| 11016 | G | A |
| 11017 | T | C |
| 11023 | A | G |
| 11078 | A | G |
| 11092 | A | G |
| 11147 | T | C |
| 11150 | G | A |
| 11167 | A | G |
| 11172 | A | G |
| 11176 | G | A |
| 11177 | C | T |
| 11215 | C | T |
| 11251 | A | G |
| 11257 | C | T |
| 11296 | C | T |
| 11299 | T | C |
| 11332 | C | T |
| 11362 | A | G |
| 11365 | T | C |
| 11377 | G | A |
| 11467 | A | G |
| 11476 | C | T |
| 11536 | C | T |
| 11590 | A | G |
| 11611 | G | A |
| 11641 | A | G |
| 11653 | A | G |
| 11654 | A | G |
| 11674 | C | T |
| 11701 | T | C |
| 11719 | G | A |
| 11722 | T | C |
| 11767 | C | T |
| 11812 | A | G |
| 11854 | T | C |
| 11884 | A | G |
| 11887 | G | A |
| 11893 | A | G |
| 11899 | T | C |
| 11909 | A | G |
| 11914 | G | A |
| 11944 | T | C |
| 11947 | A | G |
| 11959 | A | G |
| 11963 | G | A |
| 11969 | G | A |
| 12007 | G | A |
| 12049 | C | T |
| 12070 | G | A |
| 12083 | T | G |
| 12121 | T | C |
| 12134 | T | C |
| 12153 | C | T |
| 12172 | A | G |
| 12175 | T | C |
| 12234 | A | G |
| 12236 | G | A |
| 12239 | C | T |
| 12248 | A | G |
| 12308 | A | G |
| 12346 | C | T |
| 12358 | A | G |
| 12361 | A | G |
| 12372 | G | A |
| 12373 | A | G |
| 12397 | A | G |
| 12406 | G | A |
| 12414 | T | C |
| 12477 | T | C |
| 12501 | G | A |
| 12507 | A | G |
| 12519 | T | C |
| 12528 | G | A |
| 12540 | A | G |
| 12612 | A | G |
| 12630 | G | A |
| 12633 | C | T |
| 12635 | T | C |
| 12669 | C | T |
| 12672 | A | G |
| 12693 | A | G |
| 12705 | C | T |
| 12720 | A | G |
| 12738 | T | C |
| 12768 | A | G |
| 12771 | G | A |
| 12810 | A | G |
| 12822 | A | G |
| 12850 | A | G |
| 12879 | T | C |
| 12882 | C | T |
| 12930 | A | T |
| 12940 | G | A |
| 12948 | A | G |
| 12967 | A | C |
| 12972 | A | G |
| 12999 | A | G |
| 13020 | T | C |
| 13059 | C | T |
| 13068 | A | G |
| 13101 | A | C |
| 13104 | A | G |
| 13105 | A | G |
| 13135 | G | A |
| 13143 | T | C |
| 13145 | G | A |
| 13149 | A | G |
| 13194 | G | A |
| 13197 | C | T |
| 13212 | C | T |
| 13221 | A | G |
| 13263 | A | G |
| 13276 | A | G |
| 13281 | T | C |
| 13368 | G | A |
| 13440 | C | G |
| 13477 | G | A |
| 13485 | A | G |
| 13494 | C | T |
| 13500 | T | C |
| 13500 | T | G |
| 13506 | C | T |
| 13512 | A | G |
| 13563 | A | G |
| 13590 | G | A |
| 13594 | A | G |
| 13602 | T | C |
| 13611 | A | G |
| 13617 | T | C |
| 13641 | T | C |
| 13650 | C | T |
| 13651 | A | G |
| 13660 | A | G |
| 13708 | G | A |
| 13722 | A | G |
| 13734 | T | C |
| 13759 | G | A |
| 13780 | A | G |
| 13789 | T | C |
| 13803 | A | G |
| 13812 | T | C |
| 13818 | T | C |
| 13819 | T | C |
| 13827 | A | G |
| 13880 | C | A |
| 13886 | T | C |
| 13914 | C | A |
| 13924 | C | T |
| 13927 | A | T |
| 13928 | G | C |
| 13958 | G | C |
| 13965 | T | C |
| 13966 | A | G |
| 13980 | G | A |
| 14000 | T | A |
| 14016 | G | A |
| 14020 | T | C |
| 14022 | A | G |
| 14025 | T | C |
| 14034 | T | C |
| 14059 | A | G |
| 14070 | A | T |
| 14070 | A | G |
| 14088 | T | C |
| 14094 | T | C |
| 14097 | C | T |
| 14118 | A | G |
| 14128 | A | G |
| 14148 | A | G |
| 14152 | A | G |
| 14167 | C | T |
| 14178 | T | C |
| 14182 | T | C |
| 14200 | T | C |
| 14203 | A | G |
| 14209 | A | G |
| 14212 | T | C |
| 14215 | T | C |
| 14221 | T | C |
| 14233 | A | G |
| 14272 | C | G |
| 14284 | C | T |
| 14308 | T | C |
| 14311 | T | C |
| 14318 | T | C |
| 14319 | T | C |
| 14371 | T | C |
| 14374 | T | C |
| 14384 | G | C |
| 14455 | C | T |
| 14459 | G | A |
| 14470 | T | C |
| 14484 | T | C |
| 14488 | T | C |
| 14502 | T | C |
| 14560 | G | A |
| 14566 | A | G |
| 14569 | G | A |
| 14571 | T | A |
| 14580 | A | G |
| 14587 | A | G |
| 14605 | A | G |
| 14668 | C | T |
| 14693 | A | G |
| 14766 | C | T |
| 14769 | A | G |
| 14783 | T | C |
| 14793 | A | G |
| 14798 | T | C |
| 14812 | C | T |
| 14836 | A | G |
| 14861 | G | A |
| 14862 | C | T |
| 14905 | G | A |
| 14911 | C | T |
| 14971 | T | C |
| 14974 | C | G |
| 14979 | T | C |
| 15016 | C | T |
| 15034 | A | G |
| 15043 | G | A |
| 15110 | G | A |
| 15113 | A | G |
| 15115 | T | C |
| 15136 | C | T |
| 15172 | G | A |
| 15204 | T | C |
| 15217 | G | A |
| 15218 | A | C |
| 15229 | T | C |
| 15238 | C | G |
| 15244 | A | G |
| 15257 | G | A |
| 15261 | G | A |
| 15301 | G | A |
| 15317 | G | A |
| 15318 | C | T |
| 15323 | G | A |
| 15326 | A | G |
| 15346 | G | A |
| 15358 | A | G |
| 15431 | G | A |
| 15442 | A | G |
| 15452 | C | A |
| 15466 | G | A |
| 15470 | T | C |
| 15487 | A | T |
| 15497 | G | A |
| 15514 | T | C |
| 15519 | T | C |
| 15535 | C | T |
| 15607 | A | G |
| 15626 | C | T |
| 15629 | T | C |
| 15646 | C | T |
| 15661 | C | T |
| 15663 | T | C |
| 15670 | T | C |
| 15724 | A | G |
| 15731 | G | A |
| 15746 | A | G |
| 15766 | A | G |
| 15784 | T | C |
| 15793 | C | T |
| 15803 | G | A |
| 15806 | G | A |
| 15812 | G | A |
| 15824 | A | G |
| 15833 | C | T |
| 15849 | C | T |
| 15884 | G | C |
| 15900 | T | C |
| 15904 | C | T |
| 15907 | A | G |
| 15924 | A | G |
| 15927 | G | A |
| 15928 | G | A |
| 15930 | G | A |
| 15932 | T | C |
| 15939 | C | T |
| 15941 | T | C |
| 15942 | T | C |
| 15968 | T | C |
| 16017 | T | C |
| 16038 | A | G |
| 16051 | A | G |
| 16069 | C | T |
| 16071 | C | T |
| 16075 | T | C |
| 16086 | T | C |
| 16093 | T | C |
| 16108 | C | T |
| 16111 | C | T |
| 16114 | C | A |
| 16124 | T | C |
| 16126 | T | C |
| 16129 | G | A |
| 16129 | G | C |
| 16140 | T | C |
| 16144 | T | C |
| 16145 | G | A |
| 16147 | C | T |
| 16148 | C | T |
| 16153 | G | A |
| 16162 | A | G |
| 16163 | A | C |
| 16166 | A | C |
| 16167 | C | T |
| 16168 | C | T |
| 16169 | C | T |
| 16171 | A | G |
| 16172 | T | C |
| 16175 | A | G |
| 16176 | C | T |
| 16182 | A | C |
| 16183 | A | C |
| 16184 | C | T |
| 16185 | C | T |
| 16186 | C | T |
| 16187 | C | T |
| 16188 | C | A |
| 16188 | C | G |
| 16189 | T | C |
| 16192 | C | T |
| 16193 | C | T |
| 16207 | A | G |
| 16209 | T | C |
| 16212 | A | G |
| 16213 | G | A |
| 16214 | C | T |
| 16217 | T | C |
| 16219 | A | G |
| 16223 | C | T |
| 16224 | T | C |
| 16227 | A | G |
| 16229 | T | C |
| 16230 | A | G |
| 16231 | T | C |
| 16232 | C | T |
| 16234 | C | T |
| 16235 | A | G |
| 16239 | C | T |
| 16241 | A | G |
| 16242 | C | T |
| 16243 | T | C |
| 16245 | C | T |
| 16247 | A | G |
| 16249 | T | C |
| 16254 | A | C |
| 16255 | G | A |
| 16256 | C | T |
| 16257 | C | T |
| 16258 | A | G |
| 16260 | C | T |
| 16261 | C | T |
| 16264 | C | T |
| 16265 | A | C |
| 16266 | C | T |
| 16268 | C | T |
| 16270 | C | T |
| 16271 | T | C |
| 16274 | G | A |
| 16278 | C | T |
| 16284 | A | G |
| 16286 | C | G |
| 16287 | C | T |
| 16288 | T | C |
| 16290 | C | T |
| 16291 | C | T |
| 16292 | C | T |
| 16293 | A | G |
| 16294 | C | T |
| 16296 | C | T |
| 16298 | T | C |
| 16304 | T | C |
| 16309 | A | G |
| 16311 | T | C |
| 16316 | A | G |
| 16317 | A | T |
| 16318 | A | T |
| 16319 | G | A |
| 16320 | C | T |
| 16324 | T | C |
| 16325 | T | C |
| 16326 | A | G |
| 16327 | C | T |
| 16343 | A | G |
| 16344 | C | T |
| 16354 | C | T |
| 16355 | C | T |
| 16356 | T | C |
| 16357 | T | C |
| 16360 | C | T |
| 16362 | T | C |
| 16366 | C | T |
| 16368 | T | C |
| 16390 | G | A |
| 16391 | G | A |
| 16399 | A | G |
| 16438 | G | A |
| 16439 | C | A |
| 16483 | G | A |
| 16519 | T | C |
| 16527 | C | T |

Table 4 lists the nucleotide alleles identified in 48 mitochondrial genomes as compared to the Cambridge sequence.

TABLE 4
Human MtDNA Nucleotide Alleles in 48 Genomes

| | | non- |
|---|---|---|
| nucleotide | Cambridge | Cambridge |
| locus | alleles | alleles |
| 64 | C | T |
| 72 | T | C |
| 73 | A | G |
| 89 | T | C |
| 93 | A | G |
| 95 | A | C |
| 114 | C | T |
| 146 | T | C |
| 150 | C | T |
| 151 | C | T |
| 152 | T | C |
| 153 | A | G |
| 171 | G | A |
| 180 | T | C |
| 182 | C | T |
| 185 | G | A |
| 185 | G | T |
| 186 | C | A |
| 189 | A | C |
| 194 | C | T |
| 195 | T | C |
| 198 | C | T |
| 199 | T | C |
| 200 | A | G |
| 204 | T | C |
| 207 | G | A |
| 210 | A | G |
| 217 | T | C |
| 225 | G | A |
| 227 | A | G |
| 228 | G | A |
| 235 | A | G |
| 236 | T | C |
| 247 | G | A |
| 250 | T | C |
| 263 | A | G |
| 295 | C | T |
| 297 | A | G |
| 316 | G | A |
| 317 | C | G |
| 320 | C | T |
| 325 | C | T |
| 340 | C | T |
| 357 | A | G |
| 400 | T | G |
| 418 | C | T |
| 456 | C | T |
| 462 | C | T |
| 467 | C | T |
| 482 | T | C |
| 489 | T | C |
| 493 | A | G |
| 499 | G | A |
| 508 | A | G |
| 597 | C | T |
| 663 | A | G |
| 680 | T | C |
| 709 | G | A |
| 710 | T | C |
| 750 | A | G |
| 769 | G | A |
| 825 | T | A |
| 827 | A | G |
| 921 | T | C |
| 930 | G | A |
| 961 | T | C |
| 961 | T | G |
| 1018 | G | A |
| 1048 | C | T |
| 1189 | T | C |
| 1243 | T | C |
| 1290 | C | T |
| 1406 | T | C |
| 1415 | G | A |
| 1438 | A | G |
| 1442 | G | A |
| 1598 | G | A |
| 1700 | T | C |
| 1703 | C | T |
| 1706 | C | T |
| 1709 | G | A |
| 1715 | C | T |
| 1719 | G | A |
| 1736 | A | G |
| 1738 | T | C |
| 1780 | T | C |
| 1811 | A | G |
| 1888 | G | A |
| 2092 | C | T |
| 2245 | A | C |
| 2245 | A | G |
| 2308 | A | G |
| 2332 | C | T |
| 2352 | T | C |
| 2358 | A | G |
| 2416 | T | C |
| 2581 | A | G |
| 2639 | C | T |
| 2706 | A | G |
| 2758 | G | A |
| 2768 | A | G |
| 2789 | C | T |
| 2834 | C | T |
| 2857 | T | C |
| 2885 | T | C |
| 3010 | G | A |
| 3083 | T | C |
| 3197 | T | C |
| 3200 | T | A |
| 3202 | T | C |
| 3221 | A | G |
| 3308 | T | C |
| 3316 | G | A |
| 3394 | T | C |
| 3450 | C | T |
| 3480 | A | G |
| 3505 | A | G |
| 3516 | C | A |
| 3516 | C | G |
| 3547 | A | G |
| 3552 | T | A |
| 3552 | T | C |
| 3565 | A | G |
| 3594 | C | T |
| 3644 | T | C |
| 3666 | G | A |
| 3693 | G | A |
| 3720 | A | G |
| 3756 | A | G |
| 3796 | A | G |
| 3796 | A | T |
| 3796 | A | C |
| 3808 | A | G |
| 3816 | A | G |
| 3834 | G | A |
| 3843 | A | G |
| 3847 | T | C |
| 3866 | T | C |
| 3921 | C | A |
| 3970 | C | T |
| 3981 | A | G |
| 4025 | C | T |
| 4040 | C | T |
| 4044 | A | G |
| 4086 | C | T |
| 4104 | A | G |
| 4122 | A | G |
| 4123 | A | G |
| 4158 | A | G |
| 4216 | T | C |
| 4221 | C | T |
| 4225 | A | G |
| 4232 | T | C |
| 4248 | T | C |
| 4312 | C | T |
| 4336 | T | C |
| 4370 | T | C |
| 4454 | T | A |
| 4529 | A | C |
| 4529 | A | T |
| 4580 | G | A |
| 4586 | T | C |
| 4596 | G | A |
| 4646 | T | C |
| 4715 | A | G |
| 4767 | A | G |
| 4769 | A | G |
| 4820 | G | A |
| 4824 | A | G |
| 4833 | A | G |
| 4841 | G | A |
| 4883 | C | T |
| 4907 | T | C |
| 4917 | A | G |
| 4960 | C | T |
| 4977 | T | C |
| 5027 | C | T |
| 5036 | A | G |
| 5043 | G | T |
| 5046 | G | A |
| 5096 | T | C |
| 5108 | T | C |
| 5147 | G | A |
| 5153 | A | G |
| 5178 | C | A |
| 5231 | G | A |
| 5300 | C | T |
| 5331 | C | A |
| 5390 | A | G |
| 5393 | T | C |
| 5417 | G | A |
| 5426 | T | C |
| 5442 | T | C |
| 5460 | G | A |
| 5465 | T | C |
| 5471 | G | A |
| 5495 | T | C |
| 5581 | A | G |
| 5601 | C | T |
| 5603 | C | T |
| 5606 | C | T |
| 5633 | C | T |
| 5711 | A | G |
| 5773 | G | A |
| 5814 | T | C |
| 5951 | A | G |
| 5984 | A | G |
| 6026 | G | A |
| 6029 | C | T |
| 6045 | C | T |
| 6071 | T | C |
| 6152 | T | C |
| 6185 | T | C |
| 6221 | T | C |
| 6227 | T | C |
| 6257 | G | A |
| 6371 | C | T |
| 6392 | T | C |
| 6473 | C | T |
| 6491 | C | A |
| 6607 | T | C |
| 6680 | T | C |
| 6713 | C | T |
| 6734 | G | A |
| 6752 | A | G |
| 6776 | T | C |
| 6815 | T | C |
| 6827 | T | C |
| 6962 | G | A |
| 6989 | A | G |
| 7028 | C | T |
| 7052 | A | G |
| 7055 | A | G |
| 7146 | A | G |
| 7154 | A | G |
| 7175 | T | C |
| 7196 | C | A |
| 7256 | C | T |
| 7271 | A | G |
| 7274 | C | T |
| 7389 | T | C |
| 7424 | A | G |
| 7476 | C | T |
| 7521 | G | A |
| 7561 | T | C |
| 7600 | G | A |
| 7624 | T | A |
| 7664 | G | A |
| 7694 | C | T |
| 7765 | A | G |
| 7771 | A | G |
| 7864 | C | T |
| 7867 | C | T |
| 7933 | A | G |
| 7999 | T | C |
| 8027 | G | A |
| 8080 | C | T |
| 8087 | T | C |
| 8113 | C | A |
| 8142 | C | T |
| 8149 | A | G |
| 8152 | G | A |
| 8155 | G | A |
| 8185 | T | C |
| 8200 | T | C |
| 8206 | G | A |
| 8248 | A | G |
| 8251 | G | A |
| 8260 | T | C |
| 8269 | G | A |
| 8271-8279 | A | DEL |
| 8286 | T | C |
| 8298 | T | C |
| 8344 | A | G |
| 8387 | G | A |
| 8389 | A | G |
| 8392 | G | A |
| 8414 | C | T |
| 8428 | C | T |
| 8448 | T | C |
| 8460 | A | G |
| 8468 | C | T |
| 8472 | C | T |
| 8545 | G | A |
| 8553 | C | T |
| 8563 | A | G |
| 8566 | A | G |
| 8584 | G | A |
| 8618 | T | C |
| 8655 | C | T |
| 8697 | G | A |
| 8701 | A | G |
| 8705 | T | C |
| 8709 | C | T |
| 8721 | A | G |
| 8790 | G | A |
| 8794 | C | T |
| 8836 | A | G |
| 8856 | G | A |
| 8860 | A | G |
| 8875 | T | C |
| 8913 | A | G |
| 8962 | A | G |
| 8994 | G | A |
| 9042 | C | T |
| 9053 | G | A |
| 9055 | G | A |
| 9072 | A | G |
| 9077 | T | C |
| 9090 | T | C |
| 9093 | A | C |
| 9114 | A | G |
| 9120 | A | G |
| 9123 | G | A |
| 9151 | A | G |
| 9221 | A | G |
| 9237 | G | A |
| 9325 | T | C |
| 9335 | C | T |
| 9347 | A | G |
| 9355 | A | G |
| 9377 | A | G |
| 9402 | A | C |
| 9449 | C | T |
| 9456 | A | G |
| 9477 | G | A |
| 9540 | T | C |
| 9545 | A | G |
| 9548 | G | A |
| 9559 | C | G |
| 9575 | G | A |
| 9632 | A | G |
| 9682 | T | C |
| 9698 | T | C |
| 9755 | G | A |
| 9818 | C | T |
| 9822 | C | A |
| 9911 | C | T |
| 9932 | G | A |
| 9950 | T | C |
| 9957 | T | C |
| 9966 | G | A |
| 10034 | T | C |
| 10086 | A | G |
| 10086 | A | C |
| 10115 | T | C |
| 10151 | A | G |
| 10152 | G | C |
| 10172 | G | A |
| 10182 | G | C |
| 10238 | T | C |
| 10256 | T | C |
| 10310 | G | A |
| 10321 | T | C |
| 10325 | G | A |
| 10398 | A | G |
| 10400 | C | T |
| 10414 | G | T |
| 10463 | T | C |
| 10550 | A | G |
| 10586 | G | A |
| 10589 | G | A |
| 10609 | T | C |
| 10637 | C | T |
| 10646 | G | A |
| 10659 | C | T |
| 10664 | C | T |
| 10688 | G | A |
| 10790 | T | C |
| 10810 | T | C |
| 10828 | T | C |
| 10873 | T | C |
| 10876 | A | G |
| 10915 | T | C |
| 10966 | T | C |
| 10984 | C | G |
| 11002 | A | G |
| 11078 | A | G |
| 11092 | A | G |
| 11147 | T | C |
| 11167 | A | G |
| 11172 | A | G |
| 11176 | G | A |
| 11177 | C | T |
| 11215 | C | T |
| 11251 | A | G |
| 11257 | C | T |
| 11299 | T | C |
| 11332 | C | T |
| 11362 | A | G |
| 11377 | G | A |
| 11467 | A | G |
| 11476 | C | T |
| 11536 | C | T |
| 11590 | A | G |
| 11641 | A | G |
| 11674 | C | T |
| 11719 | G | A |
| 11767 | C | T |
| 11812 | A | G |
| 11854 | T | C |
| 11899 | T | C |
| 11914 | G | A |
| 11944 | T | C |
| 11947 | A | G |
| 11969 | G | A |
| 12007 | G | A |
| 12083 | T | G |
| 12121 | T | C |
| 12172 | A | G |
| 12234 | A | G |
| 12236 | G | A |
| 12308 | A | G |
| 12358 | A | G |
| 12361 | A | G |
| 12372 | G | A |
| 12373 | A | G |
| 12397 | A | G |
| 12406 | G | A |
| 12414 | T | C |
| 12501 | G | A |
| 12507 | A | G |
| 12519 | T | C |
| 12528 | G | A |
| 12540 | A | G |
| 12612 | A | G |
| 12633 | C | T |
| 12669 | C | T |
| 12672 | A | G |
| 12693 | A | G |
| 12705 | C | T |
| 12720 | A | G |
| 12738 | T | C |
| 12810 | A | G |
| 12822 | A | G |
| 12882 | C | T |
| 12930 | A | T |
| 12948 | A | G |
| 12967 | A | C |
| 12972 | A | G |
| 13020 | T | C |
| 13068 | A | G |
| 13101 | A | C |
| 13104 | A | G |
| 13105 | A | G |
| 13194 | G | A |
| 13263 | A | G |
| 13276 | A | G |
| 13368 | G | A |
| 13440 | C | G |
| 13485 | A | G |
| 13494 | C | T |
| 13500 | T | G |
| 13506 | C | T |
| 13512 | A | G |
| 13563 | A | G |
| 13590 | G | A |
| 13617 | T | C |
| 13650 | C | T |
| 13708 | G | A |
| 13734 | T | C |
| 13759 | G | A |
| 13780 | A | G |
| 13789 | T | C |
| 13803 | A | G |
| 13812 | T | C |
| 13827 | A | G |
| 13880 | C | A |
| 13886 | T | C |
| 13914 | C | A |
| 13924 | C | T |
| 13928 | G | C |
| 13958 | G | C |
| 13966 | A | G |
| 14000 | T | A |
| 14016 | G | A |
| 14034 | T | C |
| 14059 | A | G |
| 14070 | A | G |
| 14088 | T | C |
| 14118 | A | G |
| 14128 | A | G |
| 14148 | A | G |
| 14167 | C | T |
| 14178 | T | C |
| 14200 | T | C |
| 14203 | A | G |
| 14215 | T | C |
| 14221 | T | C |
| 14233 | A | G |
| 14272 | C | G |
| 14284 | C | T |
| 14308 | T | C |
| 14318 | T | C |
| 14374 | T | C |
| 14459 | G | A |
| 14470 | T | C |
| 14484 | T | C |
| 14488 | T | C |
| 14502 | T | C |
| 14560 | G | A |
| 14566 | A | G |
| 14569 | G | A |
| 14668 | C | T |
| 14693 | A | G |
| 14766 | C | T |
| 14783 | T | C |
| 14793 | A | G |
| 14798 | T | C |
| 14836 | A | G |
| 14861 | G | A |
| 14905 | G | A |
| 14911 | C | T |
| 14974 | C | G |
| 15034 | A | G |
| 15043 | G | A |
| 15110 | G | A |
| 15115 | T | C |
| 15136 | C | T |
| 15172 | G | A |
| 15204 | T | C |
| 15217 | G | A |
| 15218 | A | C |
| 15238 | C | G |
| 15257 | G | A |
| 15261 | G | A |
| 15301 | G | A |
| 15317 | G | A |
| 15318 | C | T |
| 15323 | G | A |
| 15326 | A | G |
| 15431 | G | A |
| 15442 | A | G |
| 15452 | C | A |
| 15466 | G | A |
| 15487 | A | T |
| 15497 | G | A |
| 15519 | T | C |
| 15535 | C | T |
| 15607 | A | G |
| 15661 | C | T |
| 15724 | A | G |
| 15766 | A | G |
| 15784 | T | C |
| 15793 | C | T |
| 15806 | G | A |
| 15812 | G | A |
| 15824 | A | G |
| 15833 | C | T |
| 15849 | C | T |
| 15884 | G | C |
| 15900 | T | C |
| 15904 | C | T |
| 15907 | A | G |
| 15924 | A | G |
| 15928 | G | A |
| 15930 | G | A |
| 15939 | C | T |
| 15941 | T | C |
| 15968 | T | C |
| 16017 | T | C |
| 16051 | A | G |
| 16069 | C | T |
| 16086 | T | C |
| 16093 | T | C |
| 16108 | C | T |
| 16111 | C | T |
| 16114 | C | A |
| 16124 | T | C |
| 16126 | T | C |
| 16129 | G | A |
| 16129 | G | C |
| 16145 | G | A |
| 16148 | C | T |
| 16153 | G | A |
| 16162 | A | G |
| 16163 | A | C |
| 16167 | C | T |
| 16168 | C | T |
| 16172 | T | C |
| 16176 | C | G |
| 16182 | A | C |
| 16183 | A | C |
| 16184 | C | T |
| 16185 | C | T |
| 16186 | C | T |
| 16187 | C | T |
| 16188 | C | A |
| 16188 | C | G |
| 16189 | T | C |
| 16192 | C | T |
| 16193 | C | T |
| 16212 | A | G |
| 16213 | G | A |
| 16214 | C | T |
| 16217 | T | C |
| 16219 | A | G |
| 16223 | C | T |
| 16224 | T | C |
| 16227 | A | G |
| 16229 | T | C |
| 16230 | A | G |
| 16231 | T | C |
| 16232 | C | T |
| 16234 | C | T |
| 16235 | A | G |
| 16239 | C | T |
| 16243 | T | C |
| 16245 | C | T |
| 16249 | T | C |
| 16254 | A | C |
| 16255 | G | A |
| 16256 | C | T |
| 16258 | A | G |
| 16260 | C | T |
| 16261 | C | T |
| 16264 | C | T |
| 16265 | A | C |
| 16266 | C | T |
| 16270 | C | T |
| 16274 | G | A |
| 16278 | C | T |
| 16284 | A | G |
| 16290 | C | T |
| 16291 | C | T |
| 16292 | C | T |
| 16293 | A | G |
| 16294 | C | T |
| 16296 | C | T |
| 16298 | T | C |
| 16304 | T | C |
| 16309 | A | G |
| 16311 | T | C |
| 16317 | A | T |
| 16318 | A | T |
| 16319 | G | A |
| 16320 | C | T |
| 16325 | T | C |
| 16327 | C | T |
| 16355 | C | T |
| 16356 | T | C |
| 16360 | C | T |
| 16362 | T | C |
| 16366 | C | T |
| 16368 | T | C |
| 16390 | G | A |
| 16391 | G | A |
| 16399 | A | G |
| 16519 | T | C |

### Example 2

The mtDNA sequences of Example 1 were chosen because they represent all of the major haplogroup lineages in humans. Analysis of these sequences has reaffirmed that all human mtDNAs belong to a single maternal tree, rooted in Africa (R. L. Cann et al., Nature 325:31-36 (1987); M. J. Johnson et al., (1983) Journal of Molecular Evolution 19:255-271; D. C. Wallace et al., “Global Mitochondrial DNA Variation and the Origin of Native Americans” in The Origin of Humankind, M. Aloisi, B. Battaglia, E. Carafoli, G. A. Danieli, Eds., Venice (IOS Press, 2000); M. Ingman et al., (2000) Nature 408:708-13; and D. C. Wallace et al., (1999) Gene 238:211-230). A cladogram of these mtDNA sequences is shown in FIG. 1. Haplogroups are designated on branches of the tree. A calibration of the sequence evolution rate for the coding regions of the mtDNA, based on a human-chimpanzee divergence time of 6.5 million years ago (MYA) (M. Goodman et al., (1998) Mol Phylogenet. Evol. 9:585-98), has permitted an estimate of the time to the most recent common ancestor (MRCA) of the human mtDNA phylogeny at ˜200,000 years before present (YBP), and an estimate of the time of the MRCA for each major haplogroup (Table 5).

TABLE 5
Coalescence dates for haplogroups*

| | | Time to MRCA ± | |
|---|---|---|---|
| | Sample | s.e. (×10⁻⁴ | Time to MRCA ± |
| Haplogroup | sizes | mutations per np) ^{a} | s.e. (×10³ years) ^{b} |
| chimp + human | 1 + 104 | 818.05 ± 0.75 | 6,500 |
| humans | 104 | 24.88 ± 0.90 | 198 ± 19 |
| L0 | 8 | 17.92 ± 1.87 | 142 ± 17 |
| L1 | 9 | 17.81 ± 1.77 | 142 ± 17 |
| L2 | 7 | 11.57 ± 1.30 | 91.9 ± 11.8 |
| N | 50 | 8.09 ± 0.53 | 64.3 ± 5.8 |
| A | 4 | 4.06 ± 0.92 | 32.3 ± 7.6 |
| R | 37 | 7.66 ± 0.51 | 60.9 ± 5.5 |
| HV | 15 | 3.61 ± 0.73 | 28.7 ± 6.1 |
| H | 11 | 2.40 ± 0.40 | 19.1 ± 3.4 |
| V | 3 | 1.71 ± 0.60 | 13.6 ± 4.8 |
| JT | 7 | 6.29 ± 0.74 | 50.0 ± 6.7 |
| J | 4 | 4.33 ± 0.87 | 34.4 ± 7.2 |
| T | 3 | 1.40 ± 0.55 | 11.1 ± 4.4 |
| U | 4 | 6.51 ± 0.66 | 51.7 ± 6.2 |
| M | 22 | 8.15 ± 0.74 | 64.8 ± 7.1 |
| CZ | 10 | 5.91 ± 0.87 | 47.0 ± 7.6 |
| C | 9 | 3.56 ± 0.65 | 28.3 ± 5.5 |
| D | 6 | 4.19 ± 0.67 | 33.3 ± 5.7 |
| G | 3 | 4.75 ± 0.93 | 37.7 ± 7.8 |

* The high probability of reverse mutations in the control region led us to calculate the times to the MRCAs using the entire mtDNA, excluding the control region (np 577-16023).
^{a} Based on this value we estimated the average sequence evolution rate as (1.26 ± 0.08) × 10⁻⁸ per nucleotide per year, using the HKY85 model (M. Hasegawa et al., (1985) J Mol. Evol. 22: 160-74 (1985)).
^{b} Standard errors calculated from the inverse hessian at the maximum of the likelihood do not include any uncertainty in the calibration point, and were calculated using the delta method. The coalescence times of the various haplogroups may well be underestimated because of their small sample size.

### Example 3

Inter-Continental Founder Events

The most striking feature of the mtDNA tree is the remarkable reduction in the number of mtDNA lineages that are associated with the transition from one continent to another. For example, when humans moved to Eurasia from Africa, the number of mitochondrial lineages was reduced from dozens to two lineages. While northeastern Africa encompasses the entire range of African mtDNA variation from the exclusively African haplogroups L0-L2 to the progenitors of the European and Asian mtDNA lineages, only two African mtDNA lineages, macro-haplogroups M and N, which arose about 65,000 YBP, left Africa to colonize Eurasia. Moreover, the times of the MRCAs of macro-haplogroups M and N as well as sub-macro-haplogroup R are similar, suggesting rapid population expansion associated with the colonization of Eurasia.

Similarly, when humans later moved from Central Asia to the Americas, the number of lineages was again reduced from dozens to about five. There is great mtDNA diversity in Asia, yet this diversity is substantially reduced in Siberia, and only five mtDNA haplogroups (A, B, C, D, and X), which arose in Asia about 28,000-34,000 YBP, successfully crossed the Bering land bridge to occupy the Americas. Human mtDNA haplogroup migrations are depicted in FIG. 2.

### Example 4

Further analysis demonstrated which alleles are descriptive of the major haplogroups, selected sub-haplogroups, and selected macro-haplogroups. The mtDNA nucleotide positions and the relevant alleles are shown in FIG. 3. The data is arranged as a cladogram, such that a group on the left contains all of the alleles to its right. A vertical bar designates that the alleles to the right of the bar are present in all of the groups to the left of the bar. The haplogroup data in FIG. 3 is summarized in Tables 6 and 7. The sub-haplogroup data is summarized in Tables 8 and 9. Each group contains the alleles listed below it.

TABLE 6

| L0 | L1 | L2 | L3 | C | D | E | G | Z |
|---|---|---|---|---|---|---|---|---|
| 1048T | 2352C | 325T | 2352C | 3552C | 4883T | 16227G | 4833G | 11078G |
| 3516A | 3796C | 680C | 8618C | 4715G | 5178A | | 8200C | 16185T |
| 4312T | 5951G | 2416C | 10086C | 7196A | 8414T | | 16017C | 16224C |
| 4586C | 5984G | 2758G | 10398A | 8584A | 14668T | | 16129A | 16260T |
| 5442C | 6071C | 4158G | 10819G | 9545G | 15487T | | | |
| 6185C | 9072G | 8206A | 14212C | 13263G | 16362C | 16362C | | |
| 8113A | 10586A | 9221G | 16124C | 14318C | | | | |
| 8251A | 12810G | 11944C | 16278T | 16298C | | | | 16298C |
| 9347G | 13485G | 13803G | 16362C | 16327T | | | | |
| 9402C | 3666A | 13958C | | 489C | 489C | 489C | 489C | 489C |
| 9818T | 7055G | 16278T | | 10400T | 10400T | 10400T | 10400T | 10400T |
| 10589A | 7389C | 16390G | | 14783C | 14783C | 14783C | 14783C | 14783C |
| 10664T | 13789C | | | 15043A | 15043A | 15043A | 15043A | 15043A |
| 10915C | 14178C | 15301A | 15301A | 15301A | 15301A | 15301A | 15301A | 15301A |
| 12007A | | | | | | | | |
| 13276G | | | | | | | | |
| 13506T | | | | | | | | |
| 825A | 825A | | | | | | | |
| 2758A | 2758A | | | | | | | |
| 2885C | 2885C | | | | | | | |
| 7146G | 7146G | | | | | | | |
| 8468T | 8468T | | | | | | | |
| 8655T | 8655T | | | | | | | |
| 10688A | 10688A | | | | | | | |
| 10810C | 10810C | | | | | | | |
| 13105G | 13105G | | | | | | | |
| 769A | 769A | | | | | | | |
| 1018A | 1018A | | | | | | | |
| 3594T | 3594T | | | | | | | |
| 4104G | 4104G | | | | | | | |
| 7256T | 7256T | | | | | | | |
| 7521A | 7521A | | | | | | | |
| 13650T | 13650T | | | | | | | |

TABLE 7

| A | I | W | X | B | F | Y | V | H | U | J | T |
|---|---|---|---|---|---|---|---|---|---|---|---|
| 663G | 4529T | 204C | 1719A | | 12406A | 7933G | 72C | 2706A | 3197C | 295T | 11812G |
| 16290T | 10034C | 207A | 3516G | | 16304C | 8392A | 4580A | 7028C | 4646C | 489C | 12633T |
| 16319A | 16129A | 1243C | 6221C | | | 16126C | 15904T | | 7768G | 12612G | 14233G |
| | 16391A | 5046A | 14470C | | | 16231C | 16298C | | 9055A | 13708A | 16163C |
| | | 5460A | 16189C | 16189C | | 16266T | 73A | 73A | 11332T | 16069T | 16186T |
| | | 8251A | 16278T | | | | 11719G | 11719G | 11467G | | 16189C |
| | | 8994A | | | | | 14766C | 14766C | 12308G | | 1888A |
| | | 11947G | | | | | | | 12372A | | 4917G |
| | | 15884C | | | | | | | 13104G | | 8697A |
| | | 16292T | | | | | | | 14070G | | 10463C |
| | | | | | | | | | 15907G | | 13368A |
| | | | | | | | | | 16051G | | 14905A |
| | | | | | | | | | 16129C | | 15607G |
| | | | | | | | | | 16172C | | 15928A |
| | | | | | | | | | 16189C | | 16294T |
| | | | | | | | | | 16219G | | |
| | | | | | | | | | 16224C | 4216C | 4216C |
| | | | | | | | | | 16249C | 11251G | 11251G |
| | | | | | | | | | 16270T | 15452A | 15452A |
| | | | | | | | | | 16311T | 16126C | 16126C |
| | | | | | | | | | 16318T | | |
| | | | | | | | | | 16343G | | |
| | | | | | | | | | 16356C | | |
| | | | | 12705C | 12705C | 12705C | 12705C | 12705C | 12705C | 12705C | 12705C |
| | | | | 16223C | 16223C | 16223C | 16223C | 16223C | 16223C | 16223C | 16223C |
| 8701A | 8701A | 8701A | 8701A | 8701A | 8701A | 8701A | 8701A | 8701A | 8701A | 8701A | 8701A |
| 9540T | 9540T | 9540T | 9540T | 9540T | 9540T | 9540T | 9540T | 9540T | 9540T | 9540T | 9540T |
| 10873T | 10873T | 10873T | 10873T | 10873T | 10873T | 10873T | 10873T | 10873T | 10873T | 10873T | 10873T |

TABLE 8

| L1a1 | L1a2 | L1b1 | L1b2 | L2a | L2b | L2c | L3a | L3b | L3c | L3d |
|---|---|---|---|---|---|---|---|---|---|---|
| 4586C | 8113A | 2352C | 3796C | 13803G | 4158G | 325T | 23252C | 8618C | 10086C | 10398 |
| 9818T | 8251A | | 5951G | | | 680C | 10819G | 16124C | 16124C | |
| 1048T | 1048T | | 5984G | | | 13958C | 14212C | | 16278T | |
| 3516A | 3516A | | 6071C | 2416C | 2416C | 2416C | | | 16362C | |
| 4312T | 4312T | | 9072G | 2758G | 2758G | 2758G | | | | |
| 5442C | 5442C | | 10586A | 8206A | 8206A | 8206A | | | | |
| 6185C | 6185C | | 12810G | 9221G | 9221G | 9221G | | | | |
| 9402C | 9402C | | 13485G | 11944C | 11944C | 11944C | | | | |
| 9347G | 9347G | 3666A | 3666A | 16278T | 16278T | 16278T | | | | |
| 10589A | 10589A | 7055G | 7055G | 16390G | 16390G | 16390G | | | | |
| 10664T | 10664T | 7389C | 7389C | 15301A | 15301A | 15301A | 15301A | 15301A | 15301A | 15301A |
| 10915C | 10915C | 13789C | 13789C | | | | | | | |
| 12007A | 12007A | 14178C | 14178C | | | | | | | |
| 13276G | 13276G | | | | | | | | | |
| 13506T | 13506T | | | | | | | | | |
| 825A | 825A | 825A | 825A | | | | | | | |
| 2758A | 2758A | 2758A | 2758A | | | | | | | |
| 2885C | 2885C | 2885C | 2885C | | | | | | | |
| 7146G | 7146G | 7146G | 7146G | | | | | | | |
| 8468T | 8468T | 8468T | 8468T | | | | | | | |
| 8655T | 8655T | 8655T | 8655T | | | | | | | |
| 10688A | 10688A | 10688A | 10688A | | | | | | | |
| 10810C | 10810C | 10810C | 10810C | | | | | | | |
| 13105G | 13105G | 13105G | 13105G | | | | | | | |
| 769A | 769A | 769A | 769A | 769A | 769A | 769A | | | | |
| 1018A | 1018A | 1018A | 1018A | 1018A | 1018A | 1018A | | | | |
| 3594T | 3594T | 3594T | 3594T | 3594T | 3594T | 3594T | | | | |
| 4104G | 4104G | 4104G | 4104G | 4104G | 4104G | 4104G | | | | |
| 7256T | 7256T | 7256T | 7256T | 7256T | 7256T | 7256T | | | | |
| 7521A | 7521A | 7521A | 7521A | 7521A | 7521A | 7521A | | | | |
| 13650T | 13650T | 13650T | 13650T | 13650T | 13650T | 13650T | | | | |

TABLE 9

| UK | U7 | U6 | U5 | U4 | U3 | U2 | U1 | T* | T1 |
|---|---|---|---|---|---|---|---|---|---|
| 9055A | 16318T | 16172C | 3197C | 4646C | 16343G | 15907G | 13104G | 11812G | 12633T |
| 16224C | | 16219G | 7768G | 11332T | | 16051G | 14070G | 14233G | 16163C |
| 16311T | | | 16270T | 16356C | | 16129C | 16189C | | 16186T |
| | | | | | | | 16249C | | 16189C |
| 11467G | 11467G | 11467G | 11467G | 11467G | 11467G | 11467G | 11467G | 1888A | 1888A |
| 12308G | 12308G | 12308G | 12308G | 12308G | 12308G | 12308G | 12308G | 4917G | 4917G |
| 12372A | 12372A | 12372A | 12372A | 12372A | 12372A | 12372A | 12372A | 8697A | 8697A |
| | | | | | | | | 10463C | 10463C |
| | | | | | | | | 13368A | 13368A |
| | | | | | | | | 14905A | 14905A |
| | | | | | | | | 15607G | 15607G |
| | | | | | | | | 15928A | 15928A |
| | | | | | | | | 16294T | 16294T |
| | | | | | | | | 4216C | 4216C |
| | | | | | | | | 11251G | 11251G |
| | | | | | | | | 15452A | 15452A |
| | | | | | | | | 16126C | 16126C |
| 12705C | 12705C | 12705C | 12705C | 12705C | 12705C | 12705C | 12705C | 12705C | 12705C |
| 16223C | 16223C | 16223C | 16223C | 16223C | 16223C | 16223C | 16223C | 16223C | 16223C |
| 8701A | 8701A | 8701A | 8701A | 8701A | 8701A | 8701A | 8701A | 8701A | 8701A |
| 9540T | 9540T | 9540T | 9540T | 9540T | 9540T | 9540T | 9540T | 9540T | 9540T |
| 10873T | 10873T | 10873T | 10873T | 10873T | 10873T | 10873T | 10873T | 10873T | 10873T |

### Example 5

Further analysis of the data in FIG. 3 demonstrated sets of nucleotide alleles useful for diagnosing the haplogroups. A set of nucleotide alleles useful for diagnosing all of the haplogroups and sub-haplogroups in FIG. 3 is listed in Table 10. There are many equivalent methods for diagnosing the haplogroups. Examples of methods requiring testing only or a few loci follow. Alleles are identified in human samples containing mtDNA. Haplogroup L0 can be diagnosed by identifying 4586C, 9818T, or 8113A. Haplogroup L1 can be diagnosed by identifying 825A, 2758A, 2885C, 7146G, 8468T, 8655T, 10688A, 10810C, or 13105G. Haplogroup L2 can be diagnosed by identifying 2416C, 2758G, 8206A, 9221G, 11944C, or 16390G. Haplogroup L3 can be diagnosed by identifying 10819G, 14212C, 8618C, 10086C, 16362C, 10398A, or 16124C. Haplogroup C can be diagnosed by identifying 3552C, 4715G, 7196A, 8584A, 9545G, 13263G, 14318C, or 16327T. Haplogroup D can be diagnosed by identifying 4883T, 5178A, 8414T, 14668T, or 15487T. Haplogroup E can be diagnosed by identifying 16227G. Haplogroup G can be diagnosed by identifying 4833G, 8200C, or 16017C. Haplogroup Z can be diagnosed by identifying 11078G, 16185T, or 16260T. Haplogroup A can be diagnosed by identifying 663G, 16290T, or 16319A. Haplogroup I can be diagnosed by identifying 4529T, 10034C, or 16391A. Haplogroup W can be diagnosed by identifying 204C, 207A, 1243C, 5046A, 5460A, 8994A, 11947G, 15884C, or 16292T. Haplogroup X can be diagnosed by identifying 1719A, 3516G, 6221C, or 14470C. Haplogroup F can be diagnosed by identifying 12406A or 16304C. Haplogroup Y can be diagnosed by identifying 7933G, 8392A, 16231C, or 16266T. Haplogroup U can be diagnosed by identifying 3197C, 4646C, 7768G, 9055A, 11332T, 13104G, 14070G, 15907G, 16051G, 16129C, 16172C, 16219G, 16249C, 16270T, 16311T, 16318T, 16343G, or 16356C. Haplogroup J can be diagnosed by identifying 295T, 12612G, 13708A, or 16069T. Haplogroup T can be diagnosed by identifying 11812G, 12633T, 14233G, 16163C, 16186T, 1888A, 4917G, 8697A, 10463C, 13368A, 14905A, 15607G, 15928A, or 16294T. Haplogroup V can be diagnosed by identifying 72C, 4580A, or 15904T. Haplogroup H can be diagnosed by identifying 2706A or 7028C. Diagnosis of haplogroup B is more complicated, requiring three steps. Haplogroup B can be diagnosed by identifying 16189C; and by identifying the absence of 1719A, 3516G, 6221C, 14470C, or 16278T; and by identifying the absence of 1888A, 4216C, 4917G, 8697A, 10463C, 11251G, 11467G, 12308G, 12372A, 12633T, 13104G, 13368A, 14070G, 14905A, 15452A, 15607G, 15928A, 16126C, 16163C, 16186T, 16249C, or 16294T.

TABLE 10
Nucleotide Alleles Useful for Diagnosing Human Haplogroups

| | |
|---|---|
| 72 | C |
| 204 | C |
| 207 | A |
| 295 | T |
| 663 | G |
| 825 | A |
| 1243 | C |
| 1719 | A |
| 1888 | A |
| 2416 | C |
| 2706 | A |
| 2758 | A |
| 2758 | G |
| 2885 | C |
| 3197 | C |
| 3516 | G |
| 3552 | C |
| 4216 | C |
| 4529 | T |
| 4580 | A |
| 4586 | C |
| 4646 | C |
| 4715 | G |
| 4833 | G |
| 4883 | T |
| 4917 | G |
| 5046 | A |
| 5178 | A |
| 5460 | A |
| 6221 | C |
| 7028 | C |
| 7146 | G |
| 7196 | A |
| 7768 | G |
| 7933 | G |
| 8113 | A |
| 8200 | C |
| 8206 | A |
| 8392 | A |
| 8414 | T |
| 8468 | T |
| 8584 | A |
| 8618 | C |
| 8655 | T |
| 8697 | A |
| 8994 | A |
| 9055 | A |
| 9221 | G |
| 9545 | G |
| 9818 | T |
| 10034 | C |
| 10086 | C |
| 10398 | A |
| 10463 | C |
| 10688 | A |
| 10810 | C |
| 10819 | G |
| 11078 | G |
| 11251 | G |
| 11332 | T |
| 11467 | G |
| 11812 | G |
| 11944 | C |
| 11947 | G |
| 12308 | G |
| 12372 | A |
| 12406 | A |
| 12612 | G |
| 12633 | T |
| 13104 | G |
| 13105 | G |
| 13263 | G |
| 13368 | A |
| 13708 | A |
| 14070 | G |
| 14212 | C |
| 14233 | G |
| 14318 | C |
| 14470 | C |
| 14668 | T |
| 14905 | A |
| 15301 | A |
| 15452 | A |
| 15487 | T |
| 15607 | G |
| 15884 | C |
| 15904 | T |
| 15907 | G |
| 15928 | A |
| 16017 | C |
| 16051 | G |
| 16069 | T |
| 16124 | C |
| 16126 | C |
| 16129 | C |
| 16163 | C |
| 16172 | C |
| 16185 | T |
| 16186 | T |
| 16219 | G |
| 16227 | G |
| 16231 | C |
| 16249 | C |
| 16260 | T |
| 16266 | T |
| 16270 | T |
| 16278 | T |
| 16290 | T |
| 16292 | T |
| 16294 | T |
| 16304 | C |
| 16311 | T |
| 16318 | T |
| 16319 | A |
| 16327 | T |
| 16343 | G |
| 16356 | C |
| 16362 | C |
| 16390 | G |
| 16391 | A |

Additional alleles are included in Table 11. These alleles are useful for designing equivalent methods, to those described above, for diagnosing the haplogroups. Alleles in Table 11 are useful for designing efficient methods for diagnosing macro-haplogroups. The data in Tables 10 and 11 and FIG. 3 are also useful for identifying sub-haplogroups. This invention provides a method for diagnosing sub-haplogroup L1a1 by identifying in a human sample, one of the nucleotide alleles selected from the group consisting of 4586C and 9818T. This invention provides a method for diagnosing sub-haplogroup L1a2 by identifying in a human sample, one of the nucleotide alleles selected from the group consisting of 8113A and 8251A. This invention provides a method for diagnosing sub-haplogroup L1b1 by identifying in a human sample, the nucleotide allele 2352C and one of the nucleotide alleles selected from the group consisting of 3666A, 7055G, 7389C, 13789C, and 14178C. This invention provides a method for diagnosing sub-haplogroup L1b2 by identifying in a human sample, one of the nucleotide alleles selected from the group consisting of 3796C, 5951G, 5984G, 6071C, 9072G, 10586A, 12810G, and 13485G. This invention provides a method for diagnosing sub-haplogroup L2a by identifying in a human sample the nucleotide allele 13803G. This invention provides a method for diagnosing sub-haplogroup L2b by identifying in a human sample the nucleotide allele 4158G. This invention provides a method for diagnosing sub-haplogroup L2c by identifying in a human sample, one of the nucleotide alleles selected from the group consisting of 325T, 680C, and 13958C. This invention provides a method for diagnosing sub-haplogroup L3a by identifying in a human sample, one of the nucleotide alleles selected from the group consisting of 2325C, 10819G, and 14212C. This invention provides a method for diagnosing sub-haplogroup L3b by identifying in a human sample the nucleotide allele 8618C. This invention provides a method for diagnosing sub-haplogroup L3c by identifying in a human sample the nucleotide allele 10086C. This invention provides a method for diagnosing sub-haplogroup L3d by identifying in a human sample the nucleotide allele 10398A. This invention provides a method for diagnosing sub-haplogroup Uk by identifying in a human sample, one of the nucleotide alleles selected from the group consisting of 9055A and 16311T. This invention provides a method for diagnosing sub-haplogroup U7 by identifying in a human sample the nucleotide allele 16318T. This invention provides a method for diagnosing sub-haplogroup U6 by identifying in a human sample, one of the nucleotide alleles selected from the group consisting of 16172C and 16219G. This invention provides a method for diagnosing sub-haplogroup U5 by identifying in a human sample, one of the nucleotide alleles selected from the group consisting of 3197C, 7768G, and 16270T. This invention provides a method for diagnosing sub-haplogroup U4 by identifying in a human sample, one of the nucleotide alleles selected from the group consisting of 4646C, 11332T, 16356C. This invention provides a method for diagnosing sub-haplogroup U3 by identifying in a human sample the nucleotide allele 16343G. This invention provides a method for diagnosing sub-haplogroup U2 by identifying in a human sample, one of the nucleotide alleles selected from the group consisting of 15907G, 16051G, and 16129C. This invention provides a method for diagnosing sub-haplogroup U1 by identifying in a human sample, one of the nucleotide alleles selected from the group consisting of 13104G, 14070G, 16189C, and 16249C. This invention provides a method for diagnosing sub-haplogroup T* by identifying in a human sample, one of the nucleotide alleles selected from the group consisting of 11812G and 14233G. This invention provides a method for diagnosing sub-haplogroup T1 by identifying in a human sample, one of the nucleotide alleles selected from the group consisting of 12633T, 16163C, and 16186T.

TABLE 11
Nucleotide Alleles Useful for Diagnosing
Human Haplogroups and Macro-Haplogroups

| | |
|---|---|
| 72 | C |
| 73 | A |
| 204 | C |
| 207 | A |
| 295 | T |
| 325 | T |
| 489 | C |
| 663 | G |
| 680 | C |
| 769 | A |
| 825 | A |
| 1018 | A |
| 1048 | T |
| 1243 | C |
| 1719 | A |
| 1888 | A |
| 2352 | C |
| 2416 | C |
| 2706 | A |
| 2758 | A |
| 2758 | G |
| 2885 | C |
| 3197 | C |
| 3516 | A |
| 3516 | G |
| 3552 | C |
| 3594 | T |
| 3666 | A |
| 3796 | C |
| 4104 | G |
| 4158 | G |
| 4216 | C |
| 4312 | T |
| 4529 | T |
| 4580 | A |
| 4586 | C |
| 4646 | C |
| 4715 | G |
| 4833 | G |
| 4883 | T |
| 4917 | G |
| 5046 | A |
| 5178 | A |
| 5442 | C |
| 5460 | A |
| 5951 | G |
| 5984 | G |
| 6071 | C |
| 6185 | C |
| 6221 | C |
| 7028 | C |
| 7055 | G |
| 7146 | G |
| 7196 | A |
| 7256 | T |
| 7389 | C |
| 7521 | A |
| 7768 | G |
| 7933 | G |
| 8113 | A |
| 8200 | C |
| 8206 | A |
| 8251 | A |
| 8392 | A |
| 8414 | T |
| 8468 | T |
| 8584 | A |
| 8618 | C |
| 8655 | T |
| 8697 | A |
| 8701 | A |
| 8994 | A |
| 9055 | A |
| 9072 | G |
| 9221 | G |
| 9347 | G |
| 9402 | C |
| 9540 | T |
| 9545 | G |
| 9818 | T |
| 10034 | C |
| 10086 | C |
| 10398 | A |
| 10400 | T |
| 10463 | C |
| 10586 | A |
| 10589 | A |
| 10664 | T |
| 10688 | A |
| 10810 | C |
| 10819 | G |
| 10873 | T |
| 10915 | C |
| 11078 | G |
| 11251 | G |
| 11332 | T |
| 11467 | G |
| 11719 | G |
| 11812 | G |
| 11944 | C |
| 11947 | G |
| 12007 | A |
| 12308 | G |
| 12372 | A |
| 12406 | A |
| 12612 | G |
| 12633 | T |
| 12705 | C |
| 12810 | G |
| 13104 | G |
| 13105 | G |
| 13263 | G |
| 13276 | G |
| 13368 | A |
| 13485 | G |
| 13506 | T |
| 13650 | T |
| 13708 | A |
| 13789 | C |
| 13803 | G |
| 13958 | C |
| 14070 | G |
| 14178 | C |
| 14212 | C |
| 14233 | G |
| 14318 | C |
| 14470 | C |
| 14668 | T |
| 14766 | C |
| 14783 | C |
| 14905 | A |
| 15043 | A |
| 15301 | A |
| 15452 | A |
| 15487 | T |
| 15607 | G |
| 15884 | C |
| 15904 | T |
| 15907 | G |
| 15928 | A |
| 16017 | C |
| 16051 | G |
| 16069 | T |
| 16124 | C |
| 16126 | C |
| 16129 | A |
| 16129 | C |
| 16163 | C |
| 16172 | C |
| 16185 | T |
| 16186 | T |
| 16189 | C |
| 16219 | G |
| 16223 | C |
| 16224 | C |
| 16227 | G |
| 16231 | C |
| 16249 | C |
| 16260 | T |
| 16266 | T |
| 16270 | T |
| 16278 | T |
| 16290 | T |
| 16292 | T |
| 16294 | T |
| 16298 | C |
| 16304 | C |
| 16311 | T |
| 16318 | T |
| 16319 | A |
| 16327 | T |
| 16343 | G |
| 16356 | C |
| 16362 | C |
| 16390 | G |
| 16391 | A |

An equivalent method for diagnosing a haplogroup is diagnosing haplogroup L0 by identifying the presence of one of 825A, 2758A, 2885C, 7146G, 8468T, 8655T, 10688A, 10810C, or 13105G; and identifying the absence of one of 3666A, 7055G, 7389C, 13789C, or 14178C. Other equivalent methods can be derived from the data in FIG. 3, and are within the scope of this invention.

### Example 6

Lebers Hereditary Optic Neuropathy (LHON) is a form of blindness caused by mitochondrial DNA (mtDNA) mutations. Four mutations, 3460A, 11778A, 14484C, and 14459A, account for over 90% of LHON worldwide and are designated “primary” mutations. Primary mutations strongly predispose carriers to LHON, are not found in controls, are all in Complex I genes, and do not co-occur with each other. It has been demonstrated that the 11778A and 14484C mutations occurred more frequently than expected in association with European mtDNA haplogroup J (found in 9% of European-derived mtDNAs), suggesting a synergistic interaction among mtDNA mutations increased the probability of disease expression. Sequence analysis of two Russian LHON families without primary LHON mutations, including removal of nucleotide alleles listed in Table 3, demonstrated two new complex I mutations, 3635A and 4640C. Venous blood samples were obtained from the family members. Genomic DNA was isolated from the buffy coat blood fraction using Chelex 100 (Cetus, Emberyville, Calif., USA). mtDNA was amplified by PCR in 2-3 kb fragments, purified on Centricon 100 columns, and cycle-sequenced using BigDye Terminators (ABI/Perkin Elmer Cetus) and an ABI Prism 377 automated DNA sequencer. The mutations were confirmed using mutation-specific restriction enzyme digestion following mismatched-primer PCR amplification of white blood cell mtDNA (Brown M. D. et al., (1995) Human Mutat. 6:311-325).

### Example 7

A new primary LHON mtDNA mutation, 10663C, affecting a Complex I gene was homoplasmic in 3 Caucasian LHON families, all of which belonged to haplogroup J. These 3 families were the only haplogroup J-associated LHON families (out of 17) that did not harbor a known, primary LHON mutation. Comprehensive phylogenetic analysis of haplogroup J using complete mtDNA sequences demonstrated that the 10663C variant has arisen 3 independent times on this background. This mutation was not present in over 200 non-haplogroup J European controls, 74 haplogroup J patient and control mtDNAs, or 36 putative LHON patients without primary mutations. A partial Complex I defect was found in 10663C-containing lymphoblast and cybrid mitochondria. Thus, the 10663C mutation has occurred three independent times, each time on haplogroup J and only in LHON patients without a known LHON mutation. This makes the 10663C mutation unique among all pathogenic mtDNA mutations in that it appears to require the genetic background provided by haplogroup J for expression. These results provide further evidence for the predisposing role of haplogroup J and for the paradigm of “mild” mtDNA mutations interacting in an additive way to precipitate disease expression. Europeans with the mild ND6 np 14484 and ND3 np 10663 Leber's Hereditary Optic Neuropathy (LHON) missense mutations are more prone to blindness if they also possess the mtDNA haplogroup J.

### Example 8

To assess the importance of demographic factors in inter-continental mtDNA sequence radiation, deviations from the standard neutral model were tested for in the distribution of mtDNA sequence variants using the Tajima's D and Fu and L1 D* tests (Y. X. Fu, W. H. L1, (1993) Genetics 133:693-709. and F. Tajima, (1989) Genetics 123, 585-95). The standard neutral model of population genetics assumes a random-mating population of constant size, with all mutations uniquely arising and selectively neutral. The continental frequency distribution of pairwise mtDNA sequence differences was calculated to test for rapid population expansion using the method of A. R. Rogers, H. Harpending, (1992) Mol. Biol. Evol. 9:552-569.

For the African mtDNA sequences (n=32), the results did not significantly deviate from the standard neutral model, and the frequency distribution of pairwise sequence difference counts was broad and ragged. Both of these results are consistent with the model that the African population has been relatively stable for a long time. By contrast, the non-African mtDNAs (n=72) showed a highly significant deviation from neutrality (Tajima's D=−2.43, P<0.01; Fu and L1 D*=−5.09, P<0.02), as well as a bell-shaped frequency distribution of pairwise sequence differences. Thus, these results are consistent with population expansions having distorted the frequency distribution (L. Excoffier, J. Mol. Evol. 30:125-39 (1990) and D. A. Merriwether et al. (1991) J. Mol. Evol 33:543-555).

To better define the regional distribution of these demographic influences, the Eurasian samples were divided into European and Asian plus Native American. Analysis of all European mtDNAs also revealed significant deviations from the standard neutral model (Tajima's D=−2.19, P<0.01; Fu and Li D*=−3.31, P<0.02). The distribution of pairwise sequence differences for the European mtDNAs revealed two sharp peaks, hinting at two major expansion phases. The most recent of these peaks was lost when haplogroup H and V mtDNAs were deleted from the sample. Hence, haplogroup H, which represents 40% of modern European mtDNAs (A. Torroni et al., American Journal of Human Genetics 62, 1137-1152 (1998)) and has a MRCA of 19,000 YBP, came to predominate in Europe relatively recently.

Analysis of the aggregated Asian and Native American mtDNAs (n=41) also revealed significant deviations from the standard neutral model (Tajima's D=−2.28, P<0.01, Fu and Li D*=−4.31; P<0.02) as well as revealing a broad, bell-shaped distribution of pairwise differences consistent with rapid population expansion.

When the Asian-Native American haplogroups A, B, C, D and X mtDNAs (n=26) were analyzed separately, they also showed significant deviation from neutrality for the Fu and Li D* test (D*=−2.65, P<0.05), although not for the Tajima's D test (D=−1.60, ns). Their distribution of pairwise sequence differences was also strongly uni-modal, indicating that the population expanded as people moved through Siberia and Beringia and into the Americas.

### Example 9

Variable Replacement Mutation Rates in Human mtDNA Genes

To determine if selection was an important factor in causing the sudden shifts in mtDNA sequence variation between continents, the number of non-synonymous to synonymous base substitutions was analyzed for all 13 mtDNA protein genes of those haplogroups which contributed to the colonization of each of the major continental spaces: African, European, and Native American. For example, for the “Native Americans” the mtDNAs from the Asian-Native American haplogroups A, B, C, D and X were combined. The Asian-Native American mtDNAs from the haplogroups were combined because random mutations accumulate in founder populations and those mtDNAs which prove advantageous in new environments are enriched. Hence, the founding mutations of the haplogroup are important in the continental success of the lineage. We then tested for possible selective effects during the colonization of each continent by comparing the ratio of non-synonymous versus synonymous nucleotide substitutions for each mtDNA gene. An increase in the non-synonymous to synonymous mutation ratio suggests that selection has favored the propagation of a functionally altered protein.

The comparison of the ratio of nonsynonymous to synonymous mutations, counting each change only once, revealed great variation between continents for several genes (Table 12). Marked increases in the accumulation of non-synonymous mutations were seen for ND3 in Africans, Cytb and COIII in Europeans, and ATP6 in Native Americans. The number of non-synonymous and synonymous mutations for each gene was also compared between the different continents by computing the P value using a Two-tailed Fisher Exact Test. This revealed significant differences between Africans and both Europeans and Native Americans for COIII, between Africans and Native Americans for ATP6, and between Africans and Europeans for the sum of all mtDNA genes (Table 12). Hence, this analysis supports the hypothesis that selection has played a role in shaping continental mtDNA protein variation.

TABLE 12*

| | | | | | | | | | | Two-Tail FET | | |
|---|---|---|---|---|---|---|---|---|---|---|---|---|
| | Number of Polymorphic Sites | | | | | | | | | P-value | | |
| | African | | | European | | | Native American | | | Afr | Afr | Eur |
| | N- | | | | N- | | | N- | | vs | vs | vs |
| Gene | syn | Syn | Ratio | Syn | syn | Ratio | Syn | syn | Ratio | Eur | Am | Am |
| ND1 | 10 | 17 | 0.59 | 5 | 5 | [symbol] | 4 | 4 | [symbol] | 0.71 | 0.69 | 1.00 |
| ND2 | 9 | 22 | 0.41 | 4 | 9 | 0.44 | 3 | 7 | 0.43 | 1.00 | 1.00 | 1.00 |
| ND3 | 6 | 2 | [symbol] | 1 | 3 | 0.33 | 1 | 4 | 0.25 | 0.22 | 0.10 | 1.00 |
| ND4L | 0 | 7 | 0.00 | 0 | 1 | 0.00 | 1 | 4 | 0.25 | 1.00 | 0.42 | 1.00 |
| ND4 | 4 | 35 | 0.11 | 2 | 13 | 0.15 | 3 | 12 | 0.25 | 1.00 | 0.38 | 1.00 |
| ND5 | 15 | 31 | 0.48 | 8 | 20 | 0.40 | 2 | 14 | 0.14 | 0.80 | 0.19 | 0.28 |
| ND6 | 2 | 14 | 0.14 | 1 | 6 | 0.17 | 3 | 5 | 0.60 | 1.00 | 0.29 | 0.57 |
| Cytb | 11 | 19 | 0.58 | 14 | 9 | [symbol] | 5 | 12 | 0.42 | 0.10 | 0.75 | 0.60 |
| COI | 7 | 30 | 0.23 | 0 | 9 | 0.00 | 0 | 13 | 0.00 | 0.32 | 0.17 | 1.00 |
| COII | 3 | 19 | 0.16 | 0 | 4 | 0.00 | 2 | 6 | 0.33 | 1.00 | 0.59 | 0.52 |
| COIII | 1 | 13 | 0.08 | 6 | 5 | [symbol] | 7 | 10 | 0.70 | [symbol] | [symbol] | 0.70 |
| ATP6 | 3 | 15 | 0.20 | 5 | 6 | 0.83 | 7 | 5 | [symbol] | 0.20 | [symbol] | 0.68 |
| ATP8 | 2 | 3 | 0.67 | 2 | 0 | | 1 | 3 | 0.33 | 0.43 | 1.00 | 0.40 |
| Total | 73 | 227 | 0.32 | 48 | 90 | 0.53 | 39 | 99 | 0.39 | [symbol] | 0.41 | 0.30 |

*Replacement versus synonymous mutation numbers of mtDNA genes. Rplmt = replacement mutations, ratio = rplmt/silent. FET = Fisher Exact Test. Afr = Africa, Eur = Europe, Am = Native American. The ratios of polymorphic sites in bold-italics highlight some of the higher values observed. Those in bold-italics under Two-Tailed FET indicate comparisons that are significant at the 0.05 level.

### Example 10

Since the above analysis counts each mutation only once, irrespective of its frequency within the haplogroup, it under-emphasizes the importance of nodal mutations and over-emphasizes the importance of terminal private polymorphisms. As an alternative to this approach, we calculated the corrected non-synonymous (Kₐ) and synonymous (Kₛ) mutation frequencies and then determined the relative selective constraints acting on that gene by calculating the k_{C} value {k_{C}=−1n(Kₐ/Kₛ)}. A high k_{C} value is indicative of high protein sequence conservation and low amino acid variation, while a low value is indicative of low protein conservation and high amino acid variation (N. Neckelmann et al., (1987) Proc. Natl. Acad. Sci. USA 84:7580-7584).

The k_{C} values for each human mtDNA gene were compared across the total global collection of human mtDNA sequences (FIG. 4). The ATP6 gene was the least conserved gene in the human mtDNA, though previously it had been shown to be relatively highly conserved in inter-specific comparisons (N. Neckelmann et al., (1987) Proc. Natl. Acad. Sci. USA 84:7580-7584).

### Example 11

The higher inter-specific conservation of ATP6 was confirmed by comparing the k_{C} values of human versus chimpanzee (Pan troglodytes) and bonobo (Pan paniscus); human versus eight primate species (baboon, Bomeo and Sumatran orangutan, gibbon, gorilla, lowland gorilla, bonobo, and chimpanzee); and human versus 13 diverse mammalian species (bovine, mouse, cat, dog, pig, rat, rhinoceros, horse, gibbon, gorilla, orangutan, bonobo, chimpanzee) (FIG. 3). Thus, while ATP6 is highly conserved between species, it is very poorly conserved within humans. These results are consistent with the reduced intra-specific versus inter-specific conservation observed for other genes (C. A. Wise et al., (1998) Genetics 148:409-21), and with the hypothesis that mitochondrial protein variation is accelerated in humans and other primates, as seen in cytochrome c oxidase genes (L. I. Grossman et al., (2001) Mol. Phylogenet. EvoL. 18:26-36).

### Example 12

To further investigate the possibility that individual mtDNA protein genes differ in their selective constraints in different human continental populations, k_{C} values for all 13 mtDNA protein genes from each set of continental haplogroups were calculated: African, European, and the Native American. The cumulative selective pressure that separated the mtDNAs of pairs of continents by pair-wise comparison of the k_{C} values was calculated for the genes of each mtDNA (Table 13). Comparison of mtDNA protein k_{C} values in Europeans versus Africans revealed that three genes (ND1, cytb and COIII) had significantly lower sequence conservation in Europeans. A comparison of the kc values of Native American versus African mtDNA genes revealed six genes (ND4, ND6, COII, COIII, ATP6 and ATP8) that had significantly lower sequence conservation in Native Americans. Finally, comparison of the k_{C} values of Africans versus Europeans or Native Americans revealed four mtDNA genes (ND3, ND5, cytb, and COI) had significantly lower sequence conservation in Africans. The greatest differences in k_{C} values were seen for the comparisons of COIII and ATP6 between Africans and Native Americans and for COIII between African and Europeans (Table 13).

TABLE 13*

| | | | | Native American | |
|---|---|---|---|---|---|
| | African | European | | sequences | |
| | sequences | sequences | T-test | {A, B, C, D, X} | T-test |
| GENES | (n = 32) | (n = 31) | P value | (n = 26) | P value |
| ND1 | 2.08 ± 1.18 | 0.27 ± 1.90 | P < 0.0001 | 2.07 ± 1.92 | NS |
| ND2 | 1.72 ± 1.07 | 1.57 ± 1.85 | NS | 1.81 ± 1.11 | NS |
| ND3 | 0.51 ± 1.87 | 0.91 ± 2.32 | NS | 1.70 ± 1.32 | P < 0.01 |
| ND4L | * | * | NS | 2.41 ± 3.83 | * |
| ND4 | 3.49 ± 1.34 | 3.39 ± 2.23 | NS | 2.20 ± 1.19 | P < 0.001 |
| ND5 | 1.78 ± 0.71 | 2.20 ± 1.20 | NS | 3.63 ± 3.56 | P < 0.01 |
| ND6 | 2.51 ± 1.19 | 3.13 ± 3.99 | NS | 1.15 ± 1.52 | P < 0.001 |
| Cytb | 1.89 ± 0.96 | 0.34 ± 1.51 | P < 0.0001 | 2.46 ± 1.15 | P < 0.05 |
| COI | 2.37 ± 0.95 | 3.85 ± 3.93 | P < 0.05 | * | * |
| COII | 2.73 ± 1.32 | * | * | 1.74 ± 2.12 | P < 0.05 |
| COIII | 4.65 ± 3.94 | 0.94 ± 2.08 | P < 0.0001 | 2.11 ± 1.26 | P < 0.01 |
| ATP6 | 2.31 ± 1.28 | 1.48 ± 2.28 | NS | −0.14 ± 1.34 | P < 0.0001 |
| ATP8 | 2.62 ± 1.89 | * | * | 1.25 ± 1.94 | P < 0.01 |

*Estimates of coefficients of selective constraint (k_{c}) stratified by gene and region. k_{c} values and standard deviations calculated for African, European and Asian-American haplogroups A, B, C, D and X mtDNA protein-coding genes.
* indicates that k_{c} values could not be calculated, since either Kₛ or Kₐ were 0, Haplogroup X is represented only by the Native-American sequence, the European X sequence being excluded.

Taken together, these data show that different selective forces have acted on individual mtDNA genes as humans colonized different continents. Moreover, the observed differences in mtDNA protein sequence correlate with the climatic transitions that humans would have experienced as they migrated out of tropical and sub-tropical Africa and into temperate Eurasia and arctic Siberia and Beringia. The mtDNA genes that showed the highest amino acid sequence variation between continents were COM and ATP6.

### Example 13

The nucleotide alleles in Table 3 residing in evolutionarily significant genes identified in Examples 9-12 were analyzed for evolutionary significance. Evolutionarily significant alleles reside in evolutionarily significant genes and cause amino acid changes. A list of the evolutionarily significant nucleotide alleles in ND1, ND2, ND3, ND4, ND5, ND6, Cytb, COI, COII, COIII, ATP6, and ATP8 appear in Table 14. The Cambridge nucleotide alleles in Table 14 are evolutionarily significant. These amino acid alleles, including the Cambridge alleles, are evolutionarily significant. The locations of the amino acid alleles are identified by the location of the nucleotide allele listed in Table 3. Other evolutionarily significant nucleotide alleles not listed in Table 14, include alleles at neighboring nucleotide loci that are within the same codon and code for the same amino acids that are listed in Table 14.

TABLE 14
Evolutionarily Significant Human Mitochondrial
Nucleotide and Amino Acid Allele

| | | | Non- | | |
|---|---|---|---|---|---|
| | | | Cambridge | Cambridge | Non- |
| | Genome | Cambridge | Nucl. | Amino | Cambridge |
| Gene | Location | Nucleotide | Allele | Acid | AA Allele |
| ND1 | 3308 | T | C | M | T |
| ND1 | 3316 | G | A | A | T |
| ND1 | 3394 | T | C | Y | H |
| ND1 | 3505 | A | G | T | A |
| ND1 | 3547 | A | G | I | V |
| ND1 | 3565 | A | G | T | A |
| ND1 | 3644 | T | C | V | A |
| ND1 | 3796 | A | T | T | A |
| ND1 | 3796 | A | G | T | S |
| ND1 | 3796 | A | C | T | P |
| ND1 | 3808 | A | G | T | A |
| ND1 | 3866 | T | C | I | T |
| ND1 | 4025 | C | T | T | M |
| ND1 | 4040 | C | T | T | M |
| ND1 | 4048 | G | A | D | N |
| ND1 | 4123 | A | G | I | V |
| ND1 | 4216 | T | C | Y | H |
| ND1 | 4225 | A | G | M | V |
| ND1 | 4232 | T | C | I | T |
| ND2 | 4491 | G | A | V | I |
| ND2 | 4506 | A | G | I | V |
| ND2 | 4512 | G | A | A | T |
| ND2 | 4596 | G | A | V | I |
| ND2 | 4695 | T | C | V | I |
| ND2 | 4767 | A | G | M | V |
| ND2 | 4824 | A | G | T | A |
| ND2 | 4833 | A | G | T | A |
| ND2 | 4917 | A | G | N | D |
| ND2 | 4960 | C | T | A | G |
| ND2 | 5043 | G | T | A | S |
| ND2 | 5046 | G | A | V | I |
| ND2 | 5178 | C | A | L | M |
| ND2 | 5262 | G | A | A | T |
| ND2 | 5263 | C | T | A | V |
| ND2 | 5331 | C | A | L | I |
| ND2 | 5442 | T | C | F | L |
| ND2 | 5460 | G | A | A | T |
| COI | 6150 | G | A | V | I |
| COI | 6253 | T | C | M | T |
| COI | 6324 | G | A | A | T |
| COI | 6366 | G | A | V | I |
| COI | 6607 | T | C | F | S |
| COI | 7146 | A | G | T | A |
| COI | 7257 | A | G | I | V |
| COI | 7347 | G | A | V | I |
| COI | 7389 | T | C | Y | H |
| COI | 7444 | G | A | TER | K |
| COII | 7664 | G | A | A | T |
| COII | 7673 | A | G | I | V |
| COII | 7697 | G | A | V | I |
| COII | 8027 | G | A | A | T |
| COII | 8142 | C | T | A | V |
| ATP8 | 8387 | G | A | V | M |
| ATP8 | 8414 | C | T | L | F |
| ATP8 | 8448 | T | C | M | T |
| ATP8 | 8460 | A | G | N | S |
| ATP8 | 8472 | C | T | P | L |
| ATP8 | 8553 | C | T | S | L |
| ATP6 | 8545 | G | A | A | T |
| ATP6 | 8563 | A | G | T | A |
| ATP6 | 8566 | A | G | I | V |
| ATP6 | 8584 | G | A | A | T |
| ATP6 | 8618 | T | C | I | T |
| ATP6 | 8701 | A | G | T | A |
| ATP6 | 8705 | T | C | M | T |
| ATP6 | 8764 | G | A | A | T |
| ATP6 | 8794 | C | T | H | Y |
| ATP6 | 8836 | A | G | M | V |
| ATP6 | 8860 | A | G | T | A |
| ATP6 | 8875 | T | C | F | L |
| ATP6 | 8962 | A | G | T | A |
| ATP6 | 9053 | G | A | S | N |
| ATP6 | 9055 | G | A | A | T |
| ATP6 | 9077 | T | C | I | T |
| ATP6 | 9103 | T | C | F | L |
| ATP6 | 9136 | A | G | I | V |
| ATP6 | 9151 | A | G | I | V |
| COIII | 9237 | G | A | V | M |
| COIII | 9325 | T | C | M | T |
| COIII | 9355 | A | G | N | S |
| COIII | 9456 | A | G | I | V |
| COIII | 9402 | A | C | T | P |
| COIII | 9477 | G | A | V | I |
| COIII | 9559 | C | G | P | R |
| COIII | 9591 | G | A | V | I |
| COIII | 9667 | A | G | N | S |
| COIII | 9682 | T | C | M | T |
| COIII | 9822 | C | A | L | I |
| COIII | 9957 | T | C | F | L |
| COIII | 9966 | G | A | V | I |
| ND3 | 10086 | A | G | N | D |
| ND3 | 10086 | A | C | N | H |
| ND3 | 10152 | G | C | E | Q |
| ND3 | 10182 | G | C | D | H |
| ND3 | 10197 | G | A | A | T |
| ND3 | 10321 | T | C | V | A |
| ND3 | 10398 | A | G | T | A |
| ND4L | 10609 | T | C | C | R |
| ND4 | 10816 | A | T | K | N |
| ND4 | 10920 | C | T | P | L |
| ND4 | 11016 | G | A | S | N |
| ND4 | 11078 | A | G | I | V |
| ND4 | 11150 | G | A | A | T |
| ND4 | 11172 | A | G | N | S |
| ND4 | 11177 | C | T | P | S |
| ND4 | 11654 | A | G | T | A |
| ND4 | 11909 | A | G | T | A |
| ND4 | 11963 | G | A | V | I |
| ND4 | 11969 | G | A | A | T |
| ND4 | 12083 | T | G | S | A |
| ND4 | 12134 | T | C | S | P |
| ND5 | 12346 | C | T | H | Y |
| ND5 | 12358 | A | G | T | A |
| ND5 | 12361 | A | G | T | A |
| ND5 | 12373 | A | G | T | A |
| ND5 | 12397 | A | G | T | A |
| ND5 | 12406 | T | A | V | I |
| ND5 | 12635 | T | C | I | T |
| ND5 | 12850 | A | G | I | V |
| ND5 | 12940 | G | A | A | T |
| ND5 | 12967 | A | C | T | P |
| ND5 | 13104 | A | G | I | V |
| ND5 | 13105 | A | G | I | V |
| ND5 | 13135 | G | A | A | T |
| ND5 | 13145 | G | A | S | N |
| ND5 | 13276 | A | G | M | V |
| ND5 | 13477 | G | A | A | T |
| ND5 | 13651 | A | G | T | A |
| ND5 | 13660 | A | G | N | D |
| ND5 | 13708 | G | A | A | T |
| ND5 | 13759 | G | A | A | T |
| ND5 | 13780 | A | G | I | V |
| ND5 | 13789 | T | C | Y | H |
| ND5 | 13819 | T | C | F | L |
| ND5 | 13880 | C | A | S | Y |
| ND5 | 13886 | T | C | L | P |
| ND5 | 13924 | C | T | P | S |
| ND5 | 13927 | A | T | S | C |
| ND5 | 13928 | G | C | S | T |
| ND5 | 13958 | G | C | G | A |
| ND5 | 13966 | A | G | T | A |
| ND5 | 14000 | T | A | L | Q |
| ND5 | 14059 | A | G | I | V |
| ND5 | 14128 | A | G | T | A |
| ND6 | 14178 | T | C | I | V |
| ND6 | 14272 | C | G | L | F |
| ND6 | 14318 | T | C | N | S |
| ND6 | 14319 | T | C | N | D |
| ND6 | 14384 | G | C | A | G |
| ND6 | 14459 | G | A | A | V |
| ND6 | 14484 | T | C | M | V |
| ND6 | 14502 | T | C | I | V |
| ND6 | 14571 | T | A | S | C |
| CytB | 14766 | C | T | T | I |
| CytB | 14769 | A | G | N | S |
| CytB | 14793 | A | G | H | R |
| CytB | 14798 | T | C | F | L |
| CytB | 14861 | G | A | A | T |
| CytB | 14862 | C | T | A | V |
| CytB | 14979 | T | C | I | T |
| CytB | 15110 | G | A | A | T |
| CytB | 15113 | A | G | T | A |
| CytB | 15204 | T | C | I | T |
| CytB | 15218 | A | C | T | P |
| CytB | 15218 | A | G | T | A |
| CytB | 15238 | C | G | I | M |
| CytB | 15257 | G | A | D | N |
| CytB | 15261 | G | A | S | N |
| CytB | 15317 | G | A | A | T |
| CytB | 15318 | C | T | A | V |
| CytB | 15323 | G | A | A | T |
| CytB | 15326 | A | G | T | A |
| CytB | 15431 | G | A | A | T |
| CytB | 15452 | C | A | L | I |
| CytB | 15497 | G | A | G | S |
| CytB | 15519 | T | C | L | P |
| CytB | 15663 | T | C | I | T |
| CytB | 15731 | G | A | A | T |
| CytB | 15746 | A | G | I | V |
| CytB | 15803 | G | A | V | M |
| CytB | 15806 | G | A | A | T |
| CytB | 15812 | G | A | V | M |
| CytB | 15824 | A | G | T | A |
| CytB | 15849 | C | T | T | I |
| CytB | 15884 | G | C | A | P |

A subset of the alleles in Table 14 that are associated with predispositions to physiological conditions using the methods of this invention is listed in Table 15.

TABLE 15
Amino Acid Alleles Associated with Physiological
Conditions in this Invention

| | | Nucleotide | Amino Acid | |
|---|---|---|---|---|
| | | Alleles Useful | Alleles Useful | Haplogroups |
| | Genome | for Diagnosing | for Diagnosing | Diagnosable |
| Gene | Location | Haplogroups | Haplogroups | by Alleles |
| ND1 | 3796 | C | P | (L1b2) |
| ND2 | 4833 | G | A | G |
| ND2 | 4917 | G | D | T |
| ND2 | 5046 | A | I | W |
| ND2 | 5178 | A | M | D |
| ND2 | 5442 | C | L | L0 |
| ND2 | 5460 | A | T | W |
| COI | 7146 | G | A | L0, L1 |
| COI | 7389 | C | H | L1 |
| ATP8 | 8414 | T | F | D |
| ATP6 | 8584 | A | T | C |
| ATP6 | 8618 | C | T | (L3b) |
| ATP6 | 8701 | A | T | A, I, W, X, B, |
| | | | | F, Y, U, J, T, |
| | | | | V, H |
| ATP6 | 9055 | A | T | (Uk) |
| COIII | 9402 | C | P | L0 |
| ND3 | 10086 | C | H | (L3c) |
| ND3 | 10398 | A | T | (L3d) |
| ND4 | 11078 | G | V | Z |
| ND5 | 12406 | A | I | F |
| ND5 | 13104 | G | V | (U1) |
| ND5 | 13105 | G | V | L0, L1 |
| ND5 | 13276 | G | V | L0 |
| ND5 | 13708 | A | T | J |
| ND5 | 13789 | C | H | L1 |
| ND5 | 13958 | C | A | (L2c) |
| ND6 | 14178 | C | V | L1 |
| ND6 | 14318 | C | S | C |
| CytB | 14766 | C | T | V, H |
| CytB | 15452 | A | I | J, T |
| CytB | 15884 | C | P | W |

### Example 14

Continent-Specific Amino Acid Substitutions in ATP6

To further investigate the biological significance of the human continent-specific ATP6 amino acid substitutions, the amino acid conservation for each variable human position using 39 animal species mtDNAs (12 primates, 22 other mammals, four non-mammalian vertebrates, and Drosophila) was analyzed. This revealed that many of the ATP6 substitutions that are associated with particular mtDNA haplogroups alter evolutionarily conserved, and hence potentially functionally important, amino acids.

A threonine to alanine substitution at codon 59 (T59A, nucleotide location 8701-8703) in ATP6 separates the mtDNAs of macro-haplogroup N from the rest of the World. The polar threonine at position 59 is conserved in all great apes and some old-world monkeys.

Among the haplogroups of macro-haplogroup M, the related Siberian-Native American haplogroups C and Z are delineated by an A20T (nucleotide location 8584-8586) variant. A non-polar amino acid found in this position occurs in all animal species except for Macaca, Papio, Balaenoptera and Drosophila.

Among the haplogroups of macro-haplogroup N, the non-R lineage N1b harbors two distinctive amino acid substitutions M104V (nucleotide location 8836-8838) and T146A. (nucleotides location 8962-8964) The methionine at position 104 is conserved in all mammals, and the thereon at position 146 is conserved throughout all animal mtDNAs. Moreover, the T146A substitution is within the same transmembrane α-helix as the pathogenic mutation L156R that alters the coupling efficiency of the ATP synthase and causes the NARP and Leigh syndromes (I. Trounce, S. Neill, D. C. Wallace, Proceedings of the National Academy of Sciences of the United States of America 91, 8334-8338 (1994)).

Also in macro-haplogroup A mtDNAs harbor a H90Y (nucleotide location 8794-8796) amino acid substitution. The histidine in this position is conserved in all placental mammals except Pongo, Cebus and Loxodonta and occurs within a highly conserved region. Furthermore, among the heterogeneous group of mtDNAs carrying the tRNA^{Lys}-CoII 9bp deletion and arbitrarily assigned to haplogroup B, one mtDNA harbored a F193L (nucleotide location 9103-9105) substitution. This position is conserved in all mammals except Pongo, Papio, Cebus and Erinaceus.

Since each of the MyDNA sequences used in this comparison of different species is derived from only one or two individuals, it is possible that the rare deviant cases are due to the accumulation of environmentally adaptive mutations in those species that parallel those in humans. Thus, the above ATP6 amino acid polymorphisms have the characteristics expected for evolutionary adaptive mutations.

TABLE 16

| Nucleotide | Nucleotide | |
|---|---|---|
| Locus | Alleles | WIPO code |
| 64 | CT | y |
| 72 | TC | y |
| 73 | AG | r |
| 89 | TC | y |
| 93 | AG | r |
| 95 | AC | m |
| 114 | CT | y |
| 143 | GA | r |
| 146 | TC | y |
| 150 | CT | y |
| 151 | CT | y |
| 152 | TC | y |
| 153 | AG | r |
| 171 | GA | r |
| 180 | TC | y |
| 182 | CT | y |
| 183 | AG | r |
| 185 | GAT | d |
| 185 | GAT | d |
| 186 | CA | m |
| 189 | ACG | v |
| 189 | ACG | v |
| 194 | CT | y |
| 195 | TAC | h |
| 195 | TAC | h |
| 198 | CT | y |
| 199 | TC | y |
| 200 | AG | r |
| 204 | TC | y |
| 207 | GA | r |
| 208 | TC | y |
| 210 | AG | r |
| 212 | TC | y |
| 215 | AG | r |
| 217 | TC | y |
| 225 | GA | r |
| 227 | AG | r |
| 228 | GA | r |
| 235 | AG | r |
| 236 | TC | y |
| 247 | GA | r |
| 250 | TC | y |
| 252 | TC | y |
| 263 | AG | r |
| 291 | AG | r |
| 295 | CT | y |
| 297 | AG | r |
| 316 | GA | r |
| 317 | CAG | v |
| 317 | CAG | v |
| 320 | CT | y |
| 325 | CT | y |
| 340 | CT | y |
| 357 | AG | r |
| 373 | AG | r |
| 400 | TG | k |
| 408 | TA | w |
| 418 | CT | y |
| 456 | CT | y |
| 462 | CT | y |
| 465 | CT | y |
| 467 | CT | y |
| 471 | TC | y |
| 480 | TC | y |
| 482 | TC | y |
| 489 | TC | y |
| 493 | AG | r |
| 499 | GA | r |
| 508 | AG | r |
| 593 | TC | y |
| 597 | CT | y |
| 663 | AG | r |
| 678 | TC | y |
| 680 | TC | y |
| 709 | GA | r |
| 710 | TC | y |
| 721 | TC | y |
| 750 | AG | r |
| 769 | GA | r |
| 825 | TA | w |
| 827 | AG | r |
| 850 | TC | y |
| 921 | TC | y |
| 930 | GA | r |
| 961 | TCG | b |
| 961 | TCG | b |
| 1018 | GA | r |
| 1041 | AG | r |
| 1048 | CT | y |
| 1119 | TC | y |
| 1189 | TC | y |
| 1243 | TC | y |
| 1290 | CT | y |
| 1382 | AC | m |
| 1406 | TC | y |
| 1415 | GA | r |
| 1420 | TC | y |
| 1438 | AG | r |
| 1442 | GA | r |
| 1503 | GA | r |
| 1598 | GA | r |
| 1700 | TC | y |
| 1703 | CT | y |
| 1706 | CT | y |
| 1709 | GA | r |
| 1715 | CT | y |
| 1719 | GA | r |
| 1736 | AG | r |
| 1738 | TC | y |
| 1780 | TC | y |
| 1811 | AG | r |
| 1888 | GA | r |
| 1927 | GA | r |
| 2000 | CT | y |
| 2060 | AG | r |
| 2092 | CT | y |
| 2245 | ACG | v |
| 2245 | ACG | v |
| 2263 | CA | m |
| 2308 | AG | r |
| 2332 | CT | y |
| 2352 | TC | y |
| 2358 | AG | r |
| 2380 | CT | y |
| 2416 | TC | y |
| 2483 | TC | y |
| 2581 | AG | r |
| 2639 | CT | y |
| 2650 | CT | y |
| 2706 | AG | r |
| 2755 | AG | r |
| 2758 | GA | r |
| 2768 | AG | r |
| 2789 | CT | y |
| 2792 | AG | r |
| 2834 | CT | y |
| 2836 | CA | m |
| 2857 | TC | y |
| 2863 | TC | y |
| 2885 | TC | y |
| 3010 | GA | r |
| 3083 | TC | y |
| 3197 | TC | y |
| 3200 | TA | w |
| 3202 | TC | y |
| 3204 | CT | y |
| 3206 | CT | y |
| 3221 | AG | r |
| 3290 | TC | y |
| 3308 | TC | y |
| 3316 | GA | r |
| 3372 | TC | y |
| 3394 | TC | y |
| 3438 | GA | r |
| 3450 | CT | y |
| 3480 | AG | r |
| 3505 | AG | r |
| 3513 | CT | y |
| 3516 | CGA | v |
| 3516 | CGA | v |
| 3547 | AG | r |
| 3549 | CT | y |
| 3552 | TCA | h |
| 3552 | TCA | h |
| 3565 | AG | r |
| 3594 | CT | y |
| 3644 | TC | y |
| 3666 | GA | r |
| 3693 | GA | r |
| 3699 | CT | y |
| 3720 | AG | r |
| 3756 | AG | r |
| 3796 | AGTC | n |
| 3796 | ACGT | n |
| 3808 | AG | r |
| 3816 | AG | r |
| 3834 | GA | r |
| 3843 | AG | r |
| 3847 | TC | y |
| 3866 | TC | y |
| 3918 | GA | r |
| 3921 | CA | m |
| 3927 | AG | r |
| 3970 | CT | y |
| 3981 | AG | r |
| 4025 | CT | y |
| 4040 | CT | y |
| 4044 | AG | r |
| 4048 | GA | r |
| 4086 | CT | y |
| 4104 | AG | r |
| 4117 | TC | y |
| 4122 | AG | r |
| 4123 | AG | r |
| 4158 | AG | r |
| 4203 | AG | r |
| 4216 | TC | y |
| 4221 | CT | y |
| 4225 | AG | r |
| 4232 | TC | y |
| 4248 | TC | y |
| 4312 | CT | y |
| 4336 | TC | y |
| 4370 | TC | y |
| 4388 | AG | r |
| 4454 | TA | w |
| 4491 | GA | r |
| 4506 | AG | r |
| 4508 | CT | y |
| 4512 | GA | r |
| 4529 | ATC | h |
| 4529 | ATC | h |
| 4541 | GA | r |
| 4580 | GA | r |
| 4586 | TC | y |
| 4596 | GA | r |
| 4646 | TC | y |
| 4655 | GA | r |
| 4688 | TC | y |
| 4695 | TC | y |
| 4715 | AG | r |
| 4742 | TC | y |
| 4767 | AG | r |
| 4769 | AG | r |
| 4820 | GA | r |
| 4824 | AG | r |
| 4833 | AG | r |
| 4841 | GA | r |
| 4883 | CT | y |
| 4907 | TC | y |
| 4917 | AG | r |
| 4960 | CT | y |
| 4977 | TC | y |
| 4994 | AG | r |
| 5004 | TC | y |
| 5027 | CT | y |
| 5036 | AG | r |
| 5043 | GT | k |
| 5046 | GA | r |
| 5063 | TC | y |
| 5096 | TC | y |
| 5108 | TC | y |
| 5147 | GA | r |
| 5153 | AG | r |
| 5178 | CA | m |
| 5231 | GA | r |
| 5237 | GA | r |
| 5255 | CT | y |
| 5262 | GA | r |
| 5263 | CT | y |
| 5285 | AG | r |
| 5300 | CT | y |
| 5330 | CA | m |
| 5331 | CA | m |
| 5390 | AG | r |
| 5393 | TC | y |
| 5417 | GA | r |
| 5426 | TC | y |
| 5442 | TC | y |
| 5460 | GA | r |
| 5465 | TC | y |
| 5471 | GA | r |
| 5492 | TC | y |
| 5495 | TC | y |
| 5580 | TC | y |
| 5581 | AG | r |
| 5601 | CT | y |
| 5603 | CT | y |
| 5606 | CT | y |
| 5633 | CT | y |
| 5655 | TC | y |
| 5711 | AG | r |
| 5773 | GA | r |
| 5811 | AG | r |
| 5814 | TC | y |
| 5821 | GA | r |
| 5826 | TC | y |
| 5843 | AG | r |
| 5951 | AG | r |
| 5984 | AG | r |
| 5987 | CT | y |
| 6026 | GA | r |
| 6029 | CT | y |
| 6045 | CT | y |
| 6071 | TC | y |
| 6077 | CT | y |
| 6104 | CT | y |
| 6150 | GA | r |
| 6152 | TC | y |
| 6164 | CT | y |
| 6167 | TC | y |
| 6182 | GA | r |
| 6185 | TC | y |
| 6221 | TC | y |
| 6227 | TC | y |
| 6253 | TC | y |
| 6257 | GA | r |
| 6324 | GA | r |
| 6366 | GA | r |
| 6371 | CT | y |
| 6392 | TC | y |
| 6473 | CT | y |
| 6491 | CA | m |
| 6524 | TC | y |
| 6548 | CT | y |
| 6587 | CT | y |
| 6607 | TC | y |
| 6680 | TC | y |
| 6713 | CT | y |
| 6719 | TC | y |
| 6734 | GA | r |
| 6752 | AG | r |
| 6770 | AG | r |
| 6776 | TC | y |
| 6815 | TC | y |
| 6827 | TC | y |
| 6875 | CA | m |
| 6938 | CT | y |
| 6962 | GA | r |
| 6989 | AG | r |
| 7028 | CT | y |
| 7052 | AG | r |
| 7055 | AG | r |
| 7058 | TA | w |
| 7076 | AG | r |
| 7146 | AG | r |
| 7154 | AG | r |
| 7175 | TC | y |
| 7196 | CA | m |
| 7202 | AG | r |
| 7226 | GA | r |
| 7256 | CT | y |
| 7257 | AG | r |
| 7271 | AG | r |
| 7274 | CT | y |
| 7319 | TC | y |
| 7337 | GA | r |
| 7347 | GA | r |
| 7389 | TC | y |
| 7403 | AG | r |
| 7424 | AG | r |
| 7444 | GA | r |
| 7476 | CT | y |
| 7493 | CT | y |
| 7521 | GA | r |
| 7561 | TC | y |
| 7571 | AG | r |
| 7600 | GA | r |
| 7624 | TA | w |
| 7645 | TC | y |
| 7648 | CT | y |
| 7660 | TC | y |
| 7664 | GA | r |
| 7673 | AG | r |
| 7675 | CT | y |
| 7693 | CT | y |
| 7694 | CT | y |
| 7697 | GA | r |
| 7744 | TC | y |
| 7765 | AG | r |
| 7768 | AG | r |
| 7771 | AG | r |
| 7858 | CT | y |
| 7861 | TC | y |
| 7864 | CT | y |
| 7867 | CT | y |
| 7933 | AG | r |
| 7948 | CT | y |
| 7999 | TC | y |
| 8014 | AG | r |
| 8020 | GA | r |
| 8027 | GA | r |
| 8080 | CT | y |
| 8087 | TC | y |
| 8113 | CA | m |
| 8142 | CT | y |
| 8149 | AG | r |
| 8152 | GA | r |
| 8155 | GA | r |
| 8185 | TC | y |
| 8200 | TC | y |
| 8206 | GA | r |
| 8248 | AG | r |
| 8251 | GA | r |
| 8260 | TC | y |
| 8269 | GA | r |
| 8271-8279 | accccctct/- | |
| 8286 | TC | y |
| 8292 | GA | r |
| 8298 | TC | y |
| 8344 | AG | r |
| 8387 | GA | r |
| 8389 | AG | r |
| 8392 | GA | r |
| 8404 | TC | y |
| 8414 | CT | y |
| 8428 | CT | y |
| 8448 | TC | y |
| 8460 | AG | r |
| 8468 | CT | y |
| 8472 | CT | y |
| 8473 | TC | y |
| 8485 | GA | r |
| 8545 | GA | r |
| 8553 | CT | y |
| 8563 | AG | r |
| 8566 | AG | r |
| 8577 | AG | r |
| 8584 | GA | r |
| 8618 | TC | y |
| 8655 | CT | y |
| 8697 | GA | r |
| 8701 | AG | r |
| 8703 | CT | y |
| 8705 | TC | y |
| 8709 | CT | y |
| 8721 | AG | r |
| 8733 | TC | y |
| 8764 | GA | r |
| 8781 | CA | m |
| 8784 | AG | r |
| 8790 | GA | r |
| 8793 | TC | y |
| 8794 | CT | y |
| 8805 | AG | r |
| 8836 | AG | r |
| 8838 | GA | r |
| 8856 | GA | r |
| 8860 | AG | r |
| 8875 | TC | y |
| 8877 | TC | y |
| 8911 | TC | y |
| 8913 | AG | r |
| 8928 | TC | y |
| 8943 | CT | y |
| 8962 | AG | r |
| 8994 | GA | r |
| 9042 | CT | y |
| 9053 | GA | r |
| 9055 | GA | r |
| 9072 | AG | r |
| 9077 | TC | y |
| 9090 | TC | y |
| 9093 | AC | m |
| 9103 | TC | y |
| 9114 | AG | r |
| 9120 | AG | r |
| 9123 | GA | r |
| 9136 | AG | r |
| 9151 | AG | r |
| 9156 | AG | r |
| 9174 | TC | y |
| 9221 | AG | r |
| 9237 | GA | r |
| 9242 | AG | r |
| 9248 | CT | y |
| 9263 | AG | r |
| 9272 | CT | y |
| 9296 | CT | y |
| 9311 | TC | y |
| 9325 | TC | y |
| 9335 | CT | y |
| 9347 | AG | r |
| 9355 | AG | r |
| 9356 | CT | y |
| 9377 | AG | r |
| 9402 | AC | m |
| 9449 | CT | y |
| 9456 | AG | r |
| 9477 | GA | r |
| 9509 | TC | y |
| 9536 | CT | y |
| 9540 | TC | y |
| 9545 | AG | r |
| 9548 | GA | r |
| 9554 | GA | r |
| 9559 | CG | s |
| 9575 | GA | r |
| 9591 | GA | r |
| 9599 | CT | y |
| 9632 | AG | r |
| 9647 | TC | y |
| 9667 | AG | r |
| 9682 | TC | y |
| 9698 | TC | y |
| 9755 | GA | r |
| 9818 | CT | y |
| 9822 | CA | m |
| 9824 | TA | w |
| 9911 | CT | y |
| 9932 | GA | r |
| 9950 | TC | y |
| 9957 | TC | y |
| 9966 | GA | r |
| 9977 | TC | y |
| 10034 | TC | y |
| 10086 | ACG | v |
| 10086 | ACG | v |
| 10115 | TC | y |
| 10118 | TC | y |
| 10142 | CT | y |
| 10151 | AG | r |
| 10152 | GC | s |
| 10172 | GA | r |
| 10182 | GC | s |
| 10197 | GA | r |
| 10238 | TC | y |
| 10253 | TC | y |
| 10256 | TC | y |
| 10310 | GA | r |
| 10313 | AG | r |
| 10321 | TC | y |
| 10325 | GA | r |
| 10358 | AG | r |
| 10370 | TC | y |
| 10398 | AG | r |
| 10400 | CT | y |
| 10410 | TC | y |
| 10414 | GT | k |
| 10427 | GA | r |
| 10463 | TC | y |
| 10499 | AG | r |
| 10505 | TC | y |
| 10550 | AG | r |
| 10586 | GA | r |
| 10589 | GA | r |
| 10609 | TC | y |
| 10637 | CT | y |
| 10640 | TC | y |
| 10646 | GA | r |
| 10659 | CT | y |
| 10664 | CT | y |
| 10667 | TC | y |
| 10688 | GA | r |
| 10736 | CT | y |
| 10790 | TC | y |
| 10792 | AG | r |
| 10793 | CT | y |
| 10804 | AG | r |
| 10810 | TC | y |
| 10819 | AG | r |
| 10828 | TC | y |
| 10873 | TC | y |
| 10876 | AG | r |
| 10894 | CT | y |
| 10915 | TC | y |
| 10920 | CT | y |
| 10939 | CT | y |
| 10966 | TC | y |
| 10984 | CG | s |
| 11002 | AG | r |
| 11016 | GA | r |
| 11017 | TC | y |
| 11023 | AG | r |
| 11078 | AG | r |
| 11092 | AG | r |
| 11147 | TC | y |
| 11150 | GA | r |
| 11167 | AG | r |
| 11172 | AG | r |
| 11176 | GA | r |
| 11177 | CT | y |
| 11215 | CT | y |
| 11251 | AG | r |
| 11257 | CT | y |
| 11296 | CT | y |
| 11299 | TC | y |
| 11332 | CT | y |
| 11362 | AG | r |
| 11365 | TC | y |
| 11377 | GA | r |
| 11467 | AG | r |
| 11476 | CT | y |
| 11536 | CT | y |
| 11590 | AG | r |
| 11611 | GA | r |
| 11641 | AG | r |
| 11653 | AG | r |
| 11654 | AG | r |
| 11674 | CT | y |
| 11701 | TC | y |
| 11719 | GA | r |
| 11722 | TC | y |
| 11767 | CT | y |
| 11812 | AG | r |
| 11854 | TC | y |
| 11884 | AG | r |
| 11887 | GA | r |
| 11893 | AG | r |
| 11899 | TC | y |
| 11909 | AG | r |
| 11914 | GA | r |
| 11944 | TC | y |
| 11947 | AG | r |
| 11959 | AG | r |
| 11963 | GA | r |
| 11969 | GA | r |
| 12007 | GA | r |
| 12049 | CT | y |
| 12070 | GA | r |
| 12083 | TG | k |
| 12121 | TC | y |
| 12134 | TC | y |
| 12153 | CT | y |
| 12172 | AG | r |
| 12175 | TC | y |
| 12234 | AG | r |
| 12236 | GA | r |
| 12239 | CT | y |
| 12248 | AG | r |
| 12308 | AG | r |
| 12346 | CT | y |
| 12358 | AG | r |
| 12361 | AG | r |
| 12372 | GA | r |
| 12373 | AG | r |
| 12397 | AG | r |
| 12406 | GA | r |
| 12414 | TC | y |
| 12477 | TC | y |
| 12501 | GA | r |
| 12507 | AG | r |
| 12519 | TC | y |
| 12528 | GA | r |
| 12540 | AG | r |
| 12612 | AG | r |
| 12630 | GA | r |
| 12633 | CT | y |
| 12635 | TC | y |
| 12669 | CT | y |
| 12672 | AG | r |
| 12693 | AG | r |
| 12705 | CT | y |
| 12720 | AG | r |
| 12738 | TC | y |
| 12768 | AG | r |
| 12771 | GA | r |
| 12810 | AG | r |
| 12822 | AG | r |
| 12850 | AG | r |
| 12879 | TC | y |
| 12882 | CT | y |
| 12930 | AT | w |
| 12940 | GA | r |
| 12948 | AG | r |
| 12967 | AC | m |
| 12972 | AG | r |
| 12999 | AG | r |
| 13020 | TC | y |
| 13059 | CT | y |
| 13068 | AG | r |
| 13101 | AC | m |
| 13104 | AG | r |
| 13105 | AG | r |
| 13135 | GA | r |
| 13143 | TC | y |
| 13145 | GA | r |
| 13149 | AG | r |
| 13194 | GA | r |
| 13197 | CT | y |
| 13212 | CT | y |
| 13221 | AG | r |
| 13263 | AG | r |
| 13276 | AG | r |
| 13281 | TC | y |
| 13368 | GA | r |
| 13440 | CG | s |
| 13477 | GA | r |
| 13485 | AG | r |
| 13494 | CT | y |
| 13500 | TCG | b |
| 13500 | TCG | b |
| 13506 | CT | y |
| 13512 | AG | r |
| 13563 | AG | r |
| 13590 | GA | r |
| 13594 | AG | r |
| 13602 | TC | y |
| 13611 | AG | r |
| 13617 | TC | y |
| 13641 | TC | y |
| 13650 | CT | y |
| 13651 | AG | r |
| 13660 | AG | r |
| 13708 | GA | r |
| 13722 | AG | r |
| 13734 | TC | y |
| 13759 | GA | r |
| 13780 | AG | r |
| 13789 | TC | y |
| 13803 | AG | r |
| 13812 | TC | y |
| 13818 | TC | y |
| 13819 | TC | y |
| 13827 | AG | r |
| 13880 | CA | m |
| 13886 | TC | y |
| 13914 | CA | m |
| 13924 | CT | y |
| 13927 | AT | w |
| 13928 | GC | s |
| 13958 | GC | s |
| 13965 | TC | y |
| 13966 | AG | r |
| 13980 | GA | r |
| 14000 | TA | w |
| 14016 | GA | r |
| 14020 | TC | y |
| 14022 | AG | r |
| 14025 | TC | y |
| 14034 | TC | y |
| 14059 | AG | r |
| 14070 | AGT | d |
| 14070 | AGT | d |
| 14088 | TC | y |
| 14094 | TC | y |
| 14097 | CT | y |
| 14118 | AG | r |
| 14128 | AG | r |
| 14148 | AG | r |
| 14152 | AG | r |
| 14167 | CT | y |
| 14178 | TC | y |
| 14182 | TC | y |
| 14200 | TC | y |
| 14203 | AG | r |
| 14209 | AG | r |
| 14212 | TC | y |
| 14215 | TC | y |
| 14221 | TC | y |
| 14233 | AG | r |
| 14272 | CG | s |
| 14284 | CT | y |
| 14308 | TC | y |
| 14311 | TC | y |
| 14318 | TC | y |
| 14319 | TC | y |
| 14371 | TC | y |
| 14374 | TC | y |
| 14384 | GC | s |
| 14455 | CT | y |
| 14459 | GA | r |
| 14470 | TC | y |
| 14484 | TC | y |
| 14488 | TC | y |
| 14502 | TC | y |
| 14560 | GA | r |
| 14566 | AG | r |
| 14569 | GA | r |
| 14571 | TA | w |
| 14580 | AG | r |
| 14587 | AG | r |
| 14605 | AG | r |
| 14668 | CT | y |
| 14693 | AG | r |
| 14766 | CT | y |
| 14769 | AG | r |
| 14783 | TC | y |
| 14793 | AG | r |
| 14798 | TC | y |
| 14812 | CT | y |
| 14836 | AG | r |
| 14861 | GA | r |
| 14862 | CT | y |
| 14905 | GA | r |
| 14911 | CT | y |
| 14971 | TC | y |
| 14974 | CG | s |
| 14979 | TC | y |
| 15016 | CT | y |
| 15034 | AG | r |
| 15043 | GA | r |
| 15110 | GA | r |
| 15113 | AG | r |
| 15115 | TC | y |
| 15136 | CT | y |
| 15172 | GA | r |
| 15204 | TC | y |
| 15217 | GA | r |
| 15218 | AC | m |
| 15229 | TC | y |
| 15238 | CG | s |
| 15244 | AG | r |
| 15257 | GA | r |
| 15261 | GA | r |
| 15301 | GA | r |
| 15317 | GA | r |
| 15318 | CT | y |
| 15323 | GA | r |
| 15326 | AG | r |
| 15346 | GA | r |
| 15358 | AG | r |
| 15431 | GA | r |
| 15442 | AG | r |
| 15452 | CA | m |
| 15466 | GA | r |
| 15470 | TC | y |
| 15487 | AT | w |
| 15497 | GA | r |
| 15514 | TC | y |
| 15519 | TC | y |
| 15535 | CT | y |
| 15607 | AG | r |
| 15626 | CT | y |
| 15629 | TC | y |
| 15646 | CT | y |
| 15661 | CT | y |
| 15663 | TC | y |
| 15670 | TC | y |
| 15724 | AG | r |
| 15731 | GA | r |
| 15746 | AG | r |
| 15766 | AG | r |
| 15784 | TC | y |
| 15793 | CT | y |
| 15803 | GA | r |
| 15806 | GA | r |
| 15812 | GA | r |
| 15824 | AG | r |
| 15833 | CT | y |
| 15849 | CT | y |
| 15884 | GC | s |
| 15900 | TC | y |
| 15904 | CT | y |
| 15907 | AG | r |
| 15924 | AG | r |
| 15927 | GA | r |
| 15928 | GA | r |
| 15930 | GA | r |
| 15932 | TC | y |
| 15939 | CT | y |
| 15941 | TC | y |
| 15942 | TC | y |
| 15968 | TC | y |
| 16017 | TC | y |
| 16038 | AG | r |
| 16051 | AG | r |
| 16069 | CT | y |
| 16071 | CT | y |
| 16075 | TC | y |
| 16086 | TC | y |
| 16093 | TC | y |
| 16108 | CT | y |
| 16111 | CT | y |
| 16114 | CA | m |
| 16124 | TC | y |
| 16126 | TC | y |
| 16129 | GCA | v |
| 16129 | GCA | v |
| 16140 | TC | y |
| 16144 | TC | y |
| 16145 | GA | r |
| 16147 | CT | y |
| 16148 | CT | y |
| 16153 | GA | r |
| 16162 | AG | r |
| 16163 | AC | m |
| 16166 | AC | m |
| 16167 | CT | y |
| 16168 | CT | y |
| 16169 | CT | y |
| 16171 | AG | r |
| 16172 | TC | y |
| 16175 | AG | r |
| 16176 | CT | y |
| 16182 | AC | m |
| 16183 | AC | m |
| 16184 | CT | y |
| 16185 | CT | y |
| 16186 | CT | y |
| 16187 | CT | y |
| 16188 | CAG | v |
| 16188 | CAG | v |
| 16189 | TC | y |
| 16192 | CT | y |
| 16193 | CT | y |
| 16207 | AG | r |
| 16209 | TC | y |
| 16212 | AG | r |
| 16213 | GA | r |
| 16214 | CT | y |
| 16217 | TC | y |
| 16219 | AG | r |
| 16223 | CT | y |
| 16224 | TC | y |
| 16227 | AG | r |
| 16229 | TC | y |
| 16230 | AG | r |
| 16231 | TC | y |
| 16232 | CT | y |
| 16234 | CT | y |
| 16235 | AG | r |
| 16239 | CT | y |
| 16241 | AG | r |
| 16242 | CT | y |
| 16243 | TC | y |
| 16245 | CT | y |
| 16247 | AG | r |
| 16249 | TC | y |
| 16254 | AC | m |
| 16255 | GA | r |
| 16256 | CT | y |
| 16257 | CT | y |
| 16258 | AG | r |
| 16260 | CT | y |
| 16261 | CT | y |
| 16264 | CT | y |
| 16265 | AC | m |
| 16266 | CT | y |
| 16268 | CT | y |
| 16270 | CT | y |
| 16271 | TC | y |
| 16274 | GA | r |
| 16278 | CT | y |
| 16284 | AG | r |
| 16286 | CG | s |
| 16287 | CT | y |
| 16288 | TC | y |
| 16290 | CT | y |
| 16291 | CT | y |
| 16292 | CT | y |
| 16293 | AG | r |
| 16294 | CT | y |
| 16296 | CT | y |
| 16298 | TC | y |
| 16304 | TC | y |
| 16309 | AG | r |
| 16311 | TC | y |
| 16316 | AG | r |
| 16317 | AT | w |
| 16318 | AT | w |
| 16319 | GA | r |
| 16320 | CT | y |
| 16324 | TC | y |
| 16325 | TC | y |
| 16326 | AG | r |
| 16327 | CT | y |
| 16343 | AG | r |
| 16344 | CT | y |
| 16354 | CT | y |
| 16355 | CT | y |
| 16356 | TC | y |
| 16357 | TC | y |
| 16360 | CT | y |
| 16362 | TC | y |
| 16366 | CT | y |
| 16368 | TC | y |
| 16390 | GA | r |
| 16391 | GA | r |
| 16399 | AG | r |
| 16438 | GA | r |
| 16439 | CA | m |
| 16483 | GA | r |
| 16519 | TC | y |
| 16527 | CT | y |

### Reference to Sequence Listings

SEQ ID NO:1 is a theoretical human mtDNA genome sequence containing the nucleotide alleles of this invention as listed in Table 3.

SEQ ID NO:2 is the human mtDNA reference sequence called the Cambridge Sequence (Genbank Accession No. J01415).

1-81. (canceled)

82. A method for diagnosing a haplogroup of a human comprising:
a) providing a sample comprising mitochondrial nucleic acid from said human; and
b) identifying, in said sample, the presence or absence of at least one nucleotide allele diagnostic of a haplogroup, said at least one nucleotide allele selected from the group consisting of alleles listed in Table 3.

83. The method of claim 82 wherein said haplogroup is selected from the group consisting of:
a) haplogroup A wherein method step b) comprises identifying in said sample at least one nucleotide allele selected from the group consisting of 663G, 16290T, and 16319A;
b) haplogroup C wherein method step b) comprises identifying in said sample at least one nucleotide allele selected from the group consisting of 3552C, 4715G, 7196A, 8584A, 9545G, 13263G, 14318C, and 16327T;
c) haplogroup D wherein method step b) comprises identifying in said sample at least one nucleotide allele selected from the group consisting of 4883T, 5178A, 8414T, 14668T, and 15487T;
d) haplogroup E wherein method step b) comprises identifying in said sample the nucleotide allele 16227G;
e) haplogroup F wherein method step b) comprises identifying in said sample at least one nucleotide allele selected from the group consisting of 12406A and 16304C;
f) haplogroup G wherein method step b) comprises identifying in said sample at least one nucleotide allele selected from the group consisting of 4833G, 8200C, and 16017C;
g) haplogroup H wherein method step b) comprises identifying in said sample at least one nucleotide allele selected from the group consisting of 2706A and 7028C;
h) haplogroup I wherein method step b) comprises identifying in said sample at least one nucleotide allele selected from the group consisting of 4529T, 10034C, and 16391A; and
i) haplogroup J wherein method step b) comprises identifying in said sample at least one nucleotide allele selected from the group consisting of 295T, 12612G, 13708A, and 16069T.

84. The method of claim 82 wherein said haplogroup is haplogroup B and wherein method step b) comprises:
1) identifying in said sample nucleotide allele 16189C;
2) identifying in said sample the absence of a nucleotide allele selected from the group consisting of 1719A, 3516G, 6221C, 14470C, and 16278T; and
identifying in said sample the absence of a nucleotide allele selected from the group consisting of 1888A, 4216C, 4917G, 8697A, 10463C, 11251G, 11467G, 12308G, 12372A, 12633T, 13104G, 13368A, 14070G, 14905A, 15452A, 15607G, 15928A, 16126C, 16163C, 16186T, 16249C, and 16294T.

85. The method of claim 82 wherein said haplogroup is selected from the group consisting of:
a) haplogroup T wherein method step b) comprises identifying in said sample at least one nucleotide allele selected from the group consisting of 11812G, 12633T, 14233G, 16163C, 16186T, 1888A, 4917G, 8697A, 10463C, 13368A, 14905A, 15607G, 15928A, and 16294T;
b) haplogroup U wherein method step b) comprises identifying in said sample at least one nucleotide allele selected from the group consisting of 3197C, 4646C, 7768G, 9055A, 11332T, 13104G, 14070G, 15907G, 16051G, 16129C, 16172C, 16219G, 16249C, 16270T, 16311T, 16318T, 16343G, and 16356C;
c) haplogroup V wherein method step b) comprises identifying in said sample at least one nucleotide allele selected from the group consisting of 72C, 4580A, and 15904T;
d) haplogroup W wherein method step b) comprises identifying in said sample at least one nucleotide allele selected from the group consisting of 204C, 207A, 1243C, 5046A, 5460A, 8994A, 11947G, 15884C, and 16292T;
e) haplogroup X wherein method step b) comprises identifying in said sample at least one nucleotide allele selected from the group consisting of 1719A, 3516G, 6221C, and 14470C;
f) haplogroup Y wherein method step b) comprises identifying in said sample at least one nucleotide allele selected from the group consisting of 7933G, 8392A, 16231C, and 16266T; and
g) haplogroup Z wherein method step b) comprises identifying in said sample at least one nucleotide allele selected from the group consisting of 11078G, 16185T, and 16260T.

86. The method of claim 82 wherein said haplogroup is selected from the group consisting of:
a) haplogroup L0 wherein method step b) comprises identifying in said sample at least one nucleotide allele selected from the group consisting of 4586C, 9818T, and 8113A;
b) haplogroup L1 wherein method step b) comprises identifying in said sample at least one nucleotide allele selected from the group consisting of 825A, 2758A, 2885C, 7146G, 8468T, 8655T, 10688A, 10810C, and 13105G;
c) haplogroup L2 wherein method step b) comprises identifying in said sample at least one nucleotide allele selected from the group consisting of 2416C, 2758G, 8206A, 9221G, 11944C, and 16390G; and
d) haplogroup L3 wherein method step b) comprises identifying in said sample at least one nucleotide allele selected from the group consisting of 10819G, 14212C, 8618C, 10086C, 16362C, 10398A, and 16124C.

87. The method of claim 82 wherein said identifying step is performed using an array comprising two or more isolated nucleic acid molecules attached to a substrate at a known location, each molecule having a length of about 7 to about 30 nucleotides, each molecule comprising a sequence identical with a portion of SEQ ID NO:1 containing at least one nucleotide allele at a locus selected from the group of loci consisting of those listed in column 1 of Table 3.

88. A method for identifying an evolutionarily significant gene, said method comprising:
a) providing a first set of nucleotide sequences comprising nucleic acid sequences of at least one allelic gene located in the mitochondrial genome or portion thereof from a first population;
b) providing a second set of nucleotide sequences comprising nucleic acid sequences of the corresponding at least one allelic gene located in the mitochondrial genome or portion thereof from a second population;
c) performing neutrality analysis, comprising comparing said first set to said second set to generate a data set; and
d) analyzing said data set to identify an evolutionarily significant gene.

89. The method of claim 88 wherein said first population and/or said second population comprises at least one subpopulation, said subpopulation being selected from the group consisting of macro-haplogroup, haplogroup, sub-haplogroup, and individual.

90. The method of claim 88 wherein said second set of nucleotide sequences comprises at least 100 nucleotides identical to a portion of SEQ ID NO:2.

91. The method of claim 88 wherein said evolutionarily significant gene is a mitochondrial gene selected from the group consisting of ND1, ND2, ND3, ND4, ND5, ND6, Cytb, COI, COII, COIII, ATP6, and ATP8.

92. The method of claim 88 also comprising identifying at least one evolutionarily significant nucleotide allele by identifying a sequence difference between said first and second nucleotide sequences.

93. The method of claim 92 also comprising identifying an evolutionarily significant amino acid allele by determining the evolutionarily significant amino acid allele encoded by the codon comprising said evolutionarily significant nucleotide allele.

94. The method of claim 93 also comprising identifying an amino acid allele diagnostic of a predisposition to a physiological condition by using as said first population, individuals having said physiological condition, and using as the second population, individuals not having said physiological condition.

95. A method for diagnosing an individual with a predisposition to a selected physiological condition comprising:
a) providing a sample comprising mitochondrial nucleic acid molecule from an individual;
b) providing information identifying the geographic region in which said individual resides;
c) providing information identifying a set of haplogroups native to said geographic region;
d) determining the haplogroup of said individual from said sample;
e) comparing said haplogroup of said individual to said set of haplogroups native to said geographic region; and
f) diagnosing said individual with a predisposition to said selected physiological condition if said haplogroup of said individual is not within said set of haplogroups native to said geographic region.

96. The method of claim 95 wherein said physiological condition is selected from the group consisting of energetic imbalance, metabolic disease, abnormal energy metabolism, abnormal temperature regulation, abnormal oxidative phosphorylation, abnormal electron transport, obesity, amount of body fat, diabetes, hypertension, and cardiovascular disease.

97. The method of claim 95 also comprising associating an amino acid allele with said physiological condition, said method comprising selecting an amino acid allele useful for diagnosing said haplogroup of said individual, wherein the presence of said amino acid allele is not useful for diagnosing one or more haplogroups in said set of haplogroups native to said geographical region in which said individual resides.

98. The method of claim 97 wherein said haplogroup is selected from the group consisting of:
a) haplogroup C and the amino acid allele is selected from the group consisting of ntl 8584 T and ntl 14318 S;
b) haplogroup D and the amino acid allele is selected from the group consisting of ntl 5178 M and ntl 8414F;
c) haplogroup G and the amino acid allele is selected from the group consisting of ntl 4833 A, ntl 8701 T, ntl 13708 T, and ntl 15452 I;
d) haplogroup L0 and the amino acid allele is selected from the group consisting of ntl 5442 L, ntl 7146 A, ntl 9402 P, ntl 13105 V, and ntl 13276 V;
e) haplogroup L1 and the amino acid allele is selected from the group consisting of ntl 7146 A, ntl 7389 H, ntl 13105 V, ntl 13789 H, and ntl 14178 V;
f) haplogroup T and the amino acid allele is selected from the group consisting of ntl 4917 D, ntl 8701 T, and ntl 15452 I;
g) haplogroup W and the amino acid allele is selected from the group consisting of ntl 5046 I, ntl 5460 T, ntl 8701 T, and ntl 15884 P; and
h) haplogroups V and H and the amino acid allele is selected from the group consisting of ntl 8701 T and ntl 14766 T.

99. The method of claim 97 wherein said haplogroup is selected from the group consisting of haplogroups A, I, X, B, F, Y, and U and the amino acid allele is ntl 8701 T.

100. A program storage device in which the steps of claim 95 are encoded in machine-readable form, said device also comprising a storage medium encoding said information identifying the geographic region in which said individual resides and a set of haplogroups native to said geographic region in machine readable form.

101. A storage device comprising a data set encoded in machine-readable form comprising nucleotide alleles selected from the group consisting of evolutionarily significant human mitochondrial nucleotide alleles, each said allele being associated in said storage device with encoded information identifying a physiological condition in humans.

102. The storage device of claim 101 wherein said physiological condition is selected from the group consisting of energetic imbalance, metabolic disease, abnormal energy metabolism, abnormal temperature regulation, abnormal oxidative phosphorylation, abnormal electron transport, obesity, amount of body fat, diabetes, hypertension, and cardiovascular disease.

103. The storage device of claim 101 also comprising encoded information associating each said nucleotide allele with a native geographic region.

104. A program storage device comprising the storage device of claim 101 and also comprising input means for inputting a haplogroup of an individual and a geographic region of said individual, said device further comprising program steps for diagnosing said individual as having a predisposition to a physiological condition.

105. A method for diagnosing a predisposition to LHON in a human comprising:
a) providing a sample from said human;
b) identifying in said sample nucleotide allele 10663C; and
c) identifying in said sample, nucleotide alleles encoding threonine at amino acid position 458 of gene ND5;
wherein the presence of said nucleotide alleles is diagnostic of a predisposition to LHON.

106. A method for diagnosing a predisposition to LHON in a human comprising:
a) providing a sample from said human;
b) identifying in said sample nucleotide allele 10663C; and
c) identifying in said sample at least one nucleotide allele selected
from the group consisting of 295T, 12612G, 13708A, and 16069T, wherein the presence of said nucleotide alleles is diagnostic of a predisposition to LHON.

107. A method for diagnosing a predisposition to LHON in a human comprising:
a) providing a sample from said human; and
b) identifying in said sample a nucleotide allele selected from the group consisting of 3635A and 4640C,
wherein the presence of said nucleotide alleles is diagnostic of a predisposition to LHON.

108. A method for diagnosing increased likelihood of developing blindness in a human comprising:
a) providing a sample from said human;
b) identifying in said sample a nucleotide allele selected from the group consisting of 11778A, 14484C and 10663C; and
c) identifying in said sample, nucleotide alleles encoding threonine at amino acid position 458 of gene ND5,
wherein the presence of said nucleotide alleles is diagnostic of a predisposition to develop blindness.

109. A nucleic acid array comprising two or more spots, each spot comprising a plurality of substantially identical isolated nucleic acid molecules attached to a substrate at a defined location, each molecule having a length of about 7 to about 30 nucleotides, and each molecule comprising a sequence identical with a portion of SEQ ID NO:1 containing at least one nucleotide allele at a locus selected from the group of loci consisting of those listed in column 1 of Table 3.

110. The array of claim 109 wherein at least one molecule has a sequence comprising a nucleotide allele selected from the group consisting of non-Cambridge human mtDNA nucleotide alleles of Table 3.

111. The array of claim 109 wherein at least one molecule has a sequence comprising a nucleotide allele selected from the group consisting of non-Cambridge human mtDNA nucleotide alleles of Table 4.

112. The array of claim 109 wherein at least one molecule has a sequence comprising a nucleotide allele selected from the group consisting of nucleotide alleles in nucleotide alleles useful for diagnosing human haplogroups and macro-haplogroups (Table 11).

113. The array of claim 109 comprising more than about twenty-five spots.

114. The array of claim 109 wherein said isolated nucleic acid molecules are about 20 nucleotides in length.

115. A method for determining the presence or absence of a nucleotide allele in a sample comprising:
a) providing a prepared human sample;
b) providing an array of claim 109;
c) contacting said array with and said sample under conditions allowing quantitative hybridization;
d) measuring the pattern hybridization of said sample to said array; and
e) analyzing said hybridization.

116. A program storage device comprising:
a) a machine readable storage device comprising a data set encoded in machine readable form, said data set comprising a plurality of nucleotide alleles and a haplogroup designation associated with each allele; and
b) input means for inputting a data set comprising one or more nucleotide alleles, said program storage device also comprising program steps for diagnosing a haplogroup by associating said input nucleotide alleles with an associated haplogroup, and displaying the result.
